SEQUENCE LISTING (34 sequences)

SEQ ID NO: 1 - protein, 414 aa, Mus musculus
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSPDEPLRSRKVFVGRCTEDMTAEELQQFFCQYGEVVDVFIPKPFRAFAFVTFADDKVAQSLCGEDLIIKGISVHISNAEPKHNSNRQLERSGRFGGNPGGFGNQGGFGNSRGGGAGLGNNQGGNMGGGMNFGAFSINPAMMAAAQAALQSSWGMMGMLASQQNQSGPSGNNQSQGSMQREPNQAFGSGNNSYSGSNSGAPLGWGSASNAGSGSGFNGGFGSSMDSKSSGWGM

SEQ ID NO: 2 - DNA, 7454 bp, Mus musculus
  source 1..7454: mol_type = genomic DNA
ctcggaaggccgagtggccgttctgtccttcatctgtcagtttttcagacccagctgttttcattgttgcgtttctttacttttttctatacgccgaagagcctgctagcatccgagcctctgggaggagagagcgcctgtggcttccctcggagagcgcccctcctgcagggaagccagtgggagaggccgaaggcgggcgagggcgggaggcggccctagcgccattttgtgggcacggagcggtagcgcggctgttgtcggattccttcccgtctgtgcttcctccttgtgcttcctagcagtggcctagcggagatttaagcaaagatgtctgaatatattcgggtaacagaagatgagaacgatgaacccattgaaataccatcagaagacgatgggacggtgttgctgtccacagttacagcccagtttccaggggcatgcggcctgcgctaccggaatcccgtgtctcagtgtatgagaggagtccgactggtggaaggaattctgcatgccccagatgctggctggggcaatctggtatatgttgtcaactatcccaaagataacaaaaggaaaatggatgagacagatgcttcctctgcagtgaaagtgaaaagagcagtccagaaaacatctgacctcatagtgttgggtctcccctggaaaacaactgagcaggatctgaaagactatttcagtacttttggagaggttcttatggttcaggtcaagaaagatcttaaaactggtcactcgaaagggtttggctttgttcgatttacagaatatgaaacccaagtgaaagtaatgtcacaacgacatatgatagatgggcgatggtgtgactgtaaacttcccaactctaagcaaagcccagacgagcctttgagaagcagaaaggtgtttgttggacgttgtacagaggacatgactgctgaagagcttcagcagtttttctgtcagtatggagaagtggtagatgtcttcattcccaaaccattcagagcttttgccttcgtcacctttgcagatgataaggttgcccagtctctttgtggagaggatttgatcattaaaggaatcagcgtgcatatatccaatgctgaacctaagcataatagcaatagacagttagaaagaagtggaagatttggtggtaatccaggtggctttgggaatcagggtgggtttggtaacagtagagggggtggagctggcttgggaaataaccagggtggtaatatgggtggagggatgaactttggtgcttttagcattaacccagcgatgatggctgcggctcaggcagcgttgcagagcagttggggtatgatgggcatgttagccagccagcagaaccagtcgggcccatctgggaataaccaaagccagggcagcatgcagagggaaccaaatcaggcttttggttctggaaataattcctacagtggttctaattctggtgccccccttggttgggggtcagcatcaaatgcaggatcgggcagtggttttaatgggggctttggctcgagcatggattctaagtcttctggctggggaatgtaggtggtggggggtggttagtaggttggttattaggttaggtagatttagaatggtgggattcaaatttttctaaactcatggtaagtatattgtaaaatacatatgtactaaaattttcagattggtttgttcagtgtggagtatattcagcagtatttttgacatttttctttagaaaaaaagaggggaaagctaaatgaattttataagttttgttatataaagggttaaaatactgagtgggtgaaagtgaactgctgtttgcctaattggtaaaccaacactacaattgatctcagaaggtttctctgtaatattctatcattgaaattgttaatgaattctttgcatgttcagagtagaaaccattggttagaactacattcttttctccttattttaatttgaatcccaccctatgaattttttccttaggaaaatctccatttgggagatcatgatgtcatggtgtttgattcttttggttttgtttttaacacttgtcttccttcatatacgaaagtacaatatgaagccttcatttaatctctgcagttcatctcatttcaaatgtttatggaagaagcacttcattgaaagtagtgctgtaaatattctgccataggaatacttctgtctacatgctttctcatccaagaattcgtcatcacgctgcacaggctgcgtctttgacggtgggtgttccatttttatccgctactctttatttcatggagtcgtatcaacgctatgaacgcaaggctgtgatatggaaccagaaggctgtttgaacttttgaaaccttgtgtgggattgatggtggtgccgaggcatgaaaggctagtatgagcgagaaaaggagagcgcgtgcagagacttggtggtggaaaatggatattttttaacttggagagatgtgtcactcaatcctgtggctttggtgagagagtgtgcagagagcaatgatagcaaataacgtacgaatgttttacatcaaaggacatccacatcagttggaagactttgagttttgttcttaggaaacccactttagttgaatgtgttaagtgaaatacttgtacttccctccccctctgtcaactgctgtgaatgctgtatggtgtgtgttctcctctgttactgatctggaagtgtgggaacgtgaactgaagctgatgggctgcgaacatggactgagcttgtggtgtgctttgcaggagaacttggaagcagagttcaccagtgagctcaggtgtctcaaagaagggtggaagttctcatgtctgttagctattcataagaatgctgtttgctgcagttctgtgtcctgtgcttggatgcttttttataagagttgtcattgttggaaattcttaaataaaactgatttaaataatatgtgtctttgttttgcagccctgaatgcaaagaattcatagcagttaattcccctttttgacccttttgagatggaactttcataaagtttcttggcagtagtttattttgcttcaaataaacttatttgaaaagttgtctcaagtcaaatggattcatcacctgtcatgcattgacacctgatacccagacttaattgctatttgttcttgcattgtccaaagtgaaagtttttctttggtttgtttttaatttagtttttcttaagtctgggtgaccgcacctaaaatggtaagcagttaccctctggcttgttctgagtgcctctgtgcatttgattttctatttacatgctgtataaatctccactggggaatcatgccttctaaaaatatttgggagagggcaaaagagttgatttctaatgctttgtagcagagcatatcaatgggaaagaaggttaagcacctttctgtttgggatttgaaaagtggaattaattgcaatagggatgaagtagaagaaaccaagaaaccatgtgcctgaaatacattaagaagcctgattgatagctttaagaactagtagggtgggttgtcttacctgtggcagtcttaagtgaggtaggcttttgccctcctgaatgtgggggttatgtagtgatgaatatgctcacaaaatcagattagactgtcaatgcattgttaatgtaaaagcaataatacattgattattgtacttttcctgtaactactgagaccggaggcgctccttttctaactggaagaatgggacagtttttgtgttggtagtttttcctaatgcccttacctaaatagattatgataaataggtttgtcattttgcaagttgcgtgttttaaaattttatatccgttagagacttgttatgaacacattgtttcattatacagtatcctctgtaaaaggatcgtgagttattgtaagtttttttctctgcatctaaccctgcatgatttccaaaccctgtgcatctgaattttgcattttagcactgtttgcactgttactcagcagcagtaacatggtaacattaaaatggttttcggggacctccaaagacggccaggagtcctggggtaagttacttgtcaatggcatggttttgatcccttttttacacttgttaaagacttactggtcatagaagtctttcagtgttgatcagccttttaacatgtttatggatgacatagctgtagttagttacttgccgtaaatgaggttttagaaataaactacttggcaaagatttggttttgaaagtctggtcatcaaaacgcgttcattccttagaaataatgaagaacaactctttgaaccacagttgaataaaaggttttcttgccaccaacagtttagtgtctggagtcttactggaagaaaaaaaaattctatatcatgacaatgctagaaaagttaaggtgacttatgtgggaagatgcaatatagcattttcatcctttaaaatttgagtctccaggtgggtgtggtggcccatgcttttaatcccagaattggtgtaaatgagttgtaggccagctatttccccatcttgaggcaccctgtcttgccttgttggaagagccagttaaaatcaaacatgacctttaaggtcagcatcttagcagaagagcagtttatttcaggataacttactgtttttgatacataagcaaatgactgtaccttgtacagttacggttgacttccctgagcccaacgctcacctaagaaaagtgggctgggtatagtgaaacacctgttaaggtttttggaatgatttgctaaattgcccttgtaaagggtaaaatgctgtttcgtgttctttttatctgacaatttggtgaatctggtagaacatgcctatatcccaatattctggaatggacttggtgttaatttaatagctgatctaatgtgaaggtcacacacctgctttccgcttaccttccaaaaggtattctggaaccactcagaagttactcagaaagtaagagcactttctgagctcattaagaccaaatctacacactagacacaacaagccctttgttggccagaaatggaaacagccagtataaaataagtagattgtagggaatcaaatacaacttgttttttctgtgttgggggttggccaagcactgttaaacacaaatcaaagctgatattggcaagtgtttggacctgtaacaatctcacctgctctgattttggggtaggctgtcattcttaggtttgttactaagcttcccaggtacttggcgatatgaggaacaatgattggacgatgcaaattagaaattacttattatgttctcaatccagggaatatagttgatgacttttgtgtagaccccatactggtctgccgccccacaattaatggaaccccaggaaactattcctcccacaaaccatcgctgtgtctcattatctagaaacactaatgcccccccactgtcacctctgcagctgtccttgccaccagtctctaagccagcacagagcatgttagcgcttactcttactcctggatagagcttttcatacacggcggtacatttttggtggtcagcaattggtatgtccacaaacattaggtttctagcaagaagccccttctgggttaacccccagccagccacagttccagtgaagtctgttctcattaaggatgcagcttcttttcgcggtaggcaaacaggcatgatgcttccgttgattgtgactttgttcttgagtttaatcaatgctatatcattgtcaaaaccagcaccgtgagtgtagccttcatgtataaagatttcctcgggccaggcttgagtgtaatgaggtgagagccttttgaggatgcccattcggatgttcagggaggacgctgccattcttttctcatatacagcatgagcggctgttaggacccaattgtcatgtataagtgcacctgctgctgctgtagtttgacccagcaacaagacttgccaaggaaagtcaccaggctttgcaggctgccctccaactatgcgtcctcctatagtgtgtgtggacagcccacaaactgagggataaaaaacaagtatttaatgccctacaaattgataggcatcctccctgttgagtgaggctatttaagtttttgtttagcctgctcacctccttgtaaagatcagtaagagatctcagagattgttttgctgaaagagaacagcatgagggagtctggacaggacttgtgtgcaggaggacatgacactacttagaggccaaagacaaccccctcaccacaccctgagctgcttgtttttctctttttgggctctctgggaattctgggaaagcaggaactatgatttacaaagtttctgttgtctttaaatgtaagactctaaattacaatgttgcagcaatagccaaagtgcttctggttcaaaaattggtaattttggtctggtggagccctccaaacacttttaccgttctttgcaaactgagggctcaggaatgcaacacatgttctttattgtggttgtgcactttgattaaaacttggaagccgcatgtcagccaaatacaaggctagaaaactaatttaaaccagctaacacgggggtaatgagtgtattattacctttcaattaaaaaaaaagcactctcaactgttgttggagccaattctggtaaagaaatttaagtactattaaaaggcaaattgcattaatgtttaaaaatcttgatgtcgttgaaaacaattgcttagggaaataatgaagttattagctttggggtttaatagcatttttacagagaagaaaagtaacaagagttcttggttataaatgtataaacggtttgagataatttaagaaatcatttaattttttatgcttgcctagttataaggtcaaaaacaatcaagtgcatgatgcacctagcttccgtgtggaaggggaaatgtgagcacactgttgggaaacactaagctccagcctcagccaagtgctgagctttctgcctccccagccagaccctgcctattgtctgccagctactctgtcagctatgaatctcttttataaatggcgtccattaccaggctcacaaaccggggggagtttttctcctttggagctcgtccagaatccatcagcctcacacacatatttacctgcaagtcattggaaaagcaaaaatgtttagctgtagttgtcatttgcttgaataaccccttgaaaaatgttgattcttgagcatctgtggtggggagaggtgtgtgaataaccattttacatgatttcataaataggtgtctgcattaccatgtttgcttgcaaagtggaaaccttttagatgtgtaacttgaatatgtatcaagatctcaagtgcttaatgataaggttttgacttgttaaattaaaccatttggaatatattgtg

SEQ ID NO: 3 - protein, 285 aa, Rattus norvegicus
MSEYIRVTEDENDEPIEIPSEDDGTVLLSTVTAQFPGACGLRYRNPVSQCMRGVRLVEGILHAPDAGWGNLVYVVNYPKDNKRKMDEADASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSPDEPLRSRKVFVGRCTEDMTAEELQQFFCQYGEVVDVFIPKPFRAFAFVTFADDKVAQSLCGEDLIIKGISVHISNAEPKHNSNRQLERSGRFGGKSPFGRS

SEQ ID NO: 4 - DNA, 2040 bp, Rattus norvegicus
  source 1..2040: mol_type = genomic DNA
ttttgtgggcacgaagcggtagctcggctgttgttgggttcctttccgtcttcgatccttcgttgtgcttcctagcagcggcccagtggagatttaagcaaagatgtctgaatatattcgggtaacagaagatgagaatgatgagcccattgaaataccatcagaagacgatgggacagtgttgctgtccacagttacagcccagtttccaggggcgtgtggcctgcgctaccggaatccagtgtctcagtgtatgagaggtgtccgactggtggaaggaattctgcatgccccagatgctggctggggcaatctggtctatgttgtcaactatcccaaagataacaaaaggaaaatggatgaggcggatgcttcctctgcagtgaaagtgaaaagagcagtccagaagacatctgacctcatagtgttgggtctcccctggaaaacaacagagcaggacctaaaagactacttcagtacttttggagaggttcttatggttcaggtcaagaaagatcttaaaactggtcactcaaaagggtttggctttgttcgatttacggaatatgaaactcaagtgaaagtaatgtcacagcgacatatgatagatgggcgatggtgtgactgtaaacttccaaattctaagcaaagcccagacgagcctttgagaagcagaaaggtgtttgttggacgttgtacagaggacatgactgctgaagagcttcagcagttcttctgtcagtatggagaagtggtagatgtcttcattcccaaaccattcagagcttttgcctttgttacctttgcagatgataaggttgcccagtctctttgtggagaggacttgatcattaaaggaatcagcgtgcatatatccaatgctgaacctaagcataatagcaatagacagttagaaagaagtggaagatttggtggaaaatctccatttgggagatcatgatgtcatggtgtttggttcttttggttttgtttttaacacttgtcttccttcatatacgaaagtacaatatgaagccttcatttaatctctgcagttcatctcatttcaaatgtttatggaagaagcacttcattgaaagtagtgctgtaaatattctgccataggaatacttctgtctacatgctttctcatccaagaattcgtcatcacgctgcacaggctgcgtctttgacggtgggtgttccatttttatccgctactctttatttcatggaatcgtatcaacgctatgaacgcaaggctgtgatatggaaccagaaggctgtttgaacttttgaaaccttgtgtgggattgatggtggtgccgaggcatgaaaggctagtatgagcgagaaaaggagagagcgcgtgcagagacttggtggtggaaaatggatattttttaacttggagagatgtgtccctcaatcctgtggctttggtgcgagagtgtgcagagagcaatgatagcagataactaacgtacgagtgtttctgcatcagaggacatccacgtctgttggaagactttgagttttgttcttaggaaacccacagtagctgaatgtgttaagtgaaatacttgtacttccctcccctctgtcaactgctgtgaatgctgtatggtgtgtgttctcctctgttactgatctggaagtgtgggaacgtgaactgaagctgatgggctgcgaacatggactgagcttgtggtgtgctttgcaggagaacttggaagcagagttcaccagtgagctcaggtgtctcaaagaagggtggaagttctcatgtctgttagctattcataagaatgctgtttgctgcagttctgtgtcctgtgcttggatgctttttataagagttgtcattgttggaaattcttaaataaaactgatttaaataatatgtgtctttgttttgcagccctgaatgcaaagaattcatagcagttaattcccctttttgacccttttgagatggaactttcataaagtttcttggcagtagtttattttgcttcaaataaacttatttgaaaagtt

SEQ ID NO: 5 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 6 - DNA, 4185 bp, Homo sapiens
  source 1..4185: mol_type = genomic DNA
attttgtgggagcgaagcggtggctgggctgcgcttgggtccgtcgctgcttcggtgtccctgtcgggcttcccagcagcggcctagcgggaaaagtaaaagatgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtagacagtggggttgtggttggttggtatagaatggtgggaattcaaatttttctaaactcatggtaagtatattgtaaaatacatatgtactaagaattttcaaaattggtttgttcagtgtggagtatattcagcagtatttttgacatttttctttagaaaaaggaagagctaaaggaattttataagttttgttacatgaaaggttgaaatattgagtggttgaaagtgaactgctgtttgcctgattggtaaaccaacacactacaattgatatcaaaaggtttctcctgtaatattttatccctggacttgtcaagtgaattctttgcatgttcaaaacggaaaccattgattagaactacattctttaccccttgttttaatttgaaccccaccatatggatttttttccttaagaaaatctccttttaggagatcatggtgtcacagtgtttggttcttttgttttgttttttaacacttgtctcccctcatacacaaaagtacaatatgaagccttcatttaatctctgcagttcatctcatttcaaatgtttatggaagaagcacttcattgaaagtagtgctgtaaatattctgccataggaatactgtctacatgctttctcattcaagaattcgtcatcacgcatcacaggccgcgtctttgacggtgggtgtcccatttttatccgctactctttatttcatggagtcgtatcaacgctatgaacgcaaggctgtgatatggaaccagaaggctgtctgaacttttgaaaccttgtgtgggattgatggtggtgccgaggcatgaaaggctagtatgagcgagaaaaggagagagcgcgtgcagagacttggtggtgcataatggatattttttaacttggcgagatgtgtctctcaatcctgtggctttggtgagagagtgtgcagagagcaatgatagcaaataatgtacgaatgttttttgcattcaaaggacatccacatctgttggaagacttttaagtgagtttttgttcttagataacccacattagatgaatgtgttaagtgaaatgatacttgtactccccctacccctttgtcaactgctgtgaatgctgtatggtgtgtgttctcttctgttactgatatgtaagtgtggcaatgtgaactgaagctgatgggctgagaacatggactgagcttgtggtgtgctttgcaggaggacttgaagcagagttcaccagtgagctcaggtgtctcaaagaagggtggaagttctaatgtctgttagctacccataagaatgctgtttgctgcagttctgtgtcctgtgcttggatgctttttataagagttgtcattgttggaaattcttaaataaaactgatttaaataatatgtgtctttgttttgcagccctgaatgcaaagaattcatagcagttaattccccttttttgacccttttgagatggaactttcataaagtttcttggcagtagtttattttgcttcaaataaacttatttgaaaagttgtctcaagtcaaatggattcatcacctgtcatgcattgacacctgatacccagacttaattggtatttgttcttgcattggccaaagtgaaaatttttttttttcttttgaaatctagttttgaataagtctgggtgaccgcacctaaaatggtaagcagtaccctccggctttttcttagtgcctctgtgcatttgggtgatgttctatttacatggcctgtgtaaatctccattgggaagtcatgccttctaaaaagattcttatttgggggagtgggcaaaatgttgattattttctaatgctttgtagcaaagcatatcaattgaaaagggaatatcagcaccttcctagtttgggatttgaaaagtggaattaattgcagtagggataaagtagaagaaaccacaaattatcttgtgcctgaaatccattaagaggcctgatagctttaagaattagggtgggttgtctgtctggaagtgttaagtggaatgggctttgtcctccaggaggtgggggaatgtggtaacattgaatacagttgaataaaatcgcttacaaaactcacactctcacaatgcattgttaagtatgtaaaagcaataacattgattctctgttgtacttttttgtaactaattctgtgagagttgagctcattttctagttggaagaatgtgatatttgttgtgttggtagtttacctaatgcccttacctaattagattatgataaataggtttgtcattttgcaagttacataaacatttatcaatgaagtcatcctttagacttgtaatcgccacattgtttcattattcagtttcctctgtaaagggatcttgagttgttttaattttttttttctgcatctgaatctgcatgatttccaaaccctgtaccatctgaattttgcattttagcacttgcactattactcagcagcagtaacatggtaacacttaaaatggtactcggggacctccaaagactaaactgacaagccttcaaggagcccaggggtaagttaacttgtcaacggcatggtttaatcccttctttacacttgtgtaaatttcagttactggtcatagaaggctttcaatgttgagtggccttttattaacatgtttatggtactgcatagatacgggtatttattttaccctaagaagattttgaagtttaaaagtacttaaactatttggcaaagatttgtttttaaaaatctatttggtcaatctaaatgcattcattctaaaaaattttttgaaccagataaataaaatttttttttgacaccaca

SEQ ID NO: 7 - protein, 6 aa, synthetic construct
  REGION 1..6: note = RRM1 RNP2 consensus sequence (Protein)
LIVLGL

SEQ ID NO: 8 - protein, 8 aa, synthetic construct
  REGION 1..8: note = RRM1 RNP1 consensus sequence
KGFGFVRF

SEQ ID NO: 9 - protein, 6 aa, synthetic construct
  REGION 1..6: note = RRM2 RNP2 consensus sequence
VFVGRC

SEQ ID NO: 10 - protein, 7 aa, synthetic construct
  REGION 1..7: note = RRM2 RNP1 consensus sequence
RAFAFVT

SEQ ID NO: 11 - protein, 344 aa, Escherichia phage P1
MVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 12 - DNA, 560 bp, synthetic construct
  misc_feature 1..560: note = CMV early enhancer/chicken beta acting CAG promoter
  source 1..560: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctata

SEQ ID NO: 13 - DNA, 448 bp, Homo sapiens
  source 1..448: mol_type = genomic DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 14 - DNA, 705 bp, synthetic construct
  misc_feature 1..705: note = mCherry
  source 1..705: mol_type = other DNA
gtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 15 - DNA, 477 bp, synthetic construct
  misc_feature 1..477: note = poly A signal
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 16 - DNA, 141 bp, synthetic construct
  misc_feature 1..141: note = AAV 5' ITR sequence
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 17 - DNA, 141 bp, synthetic construct
  misc_feature 1..141: note = AAV 3' ITR
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 18 - DNA, 6224 bp, synthetic construct
  misc_feature 1..6224: note = AAV-PHP.eB-hSyn-Cre plasmid
  source 1..6224: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggatcccgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcaccatggtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggacggaagcggagctactaacttcagcctgctgaagcaggctggagacgtggaggagaaccctggacctgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 19 - DNA, 6939 bp, synthetic construct
  misc_feature 1..6939: note = AAV-PHP.eB-CAG-Cre
  source 1..6939: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaccatggtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggacggaagcggagctactaacttcagcctgctgaagcaggctggagacgtggaggagaaccctggacctgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 20 - DNA, 22 bp, synthetic construct
  misc_feature 1..22: note = Sortilin1 E16/19 forward primer
  source 1..22: mol_type = other DNA
gcatgagttagagttctgtctg

SEQ ID NO: 21 - DNA, 21 bp, synthetic construct
  misc_feature 1..21: note = Sortilin1 E16/19 reverse primer
  source 1..21: mol_type = other DNA
cttccgccacagacatatttc

SEQ ID NO: 22 - DNA, 21 bp, synthetic construct
  misc_feature 1..21: note = Dnajc5 E3/5 forward primer
  source 1..21: mol_type = other DNA
ctacttcgtactctccagctg

SEQ ID NO: 23 - DNA, 21 bp, synthetic construct
  misc_feature 1..21: note = Dnajc5 E3/5 reverse primer
  source 1..21: mol_type = other DNA
gatgctggctgtatgacgatc

SEQ ID NO: 24 - DNA, 21 bp, synthetic construct
  misc_feature 1..21: note = Poldip3 E2/4 forward primer
  source 1..21: mol_type = other DNA
gagaagatcagcttgaagagg

SEQ ID NO: 25 - DNA, 21 bp, synthetic construct
  misc_feature 1..21: note = Poldip3 E2/4 reverse primer
  source 1..21: mol_type = other DNA
caccacaatgtcatcatcttc

SEQ ID NO: 26 - DNA, 22 bp, synthetic construct
  misc_feature 1..22: note = Adnp2 E2/3 forward primer
  source 1..22: mol_type = other DNA
gcagaatcttgacaacatcagg

SEQ ID NO: 27 - DNA, 21 bp, synthetic construct
  misc_feature 1..21: note = Adnp2 E2/3 reverse primer
  source 1..21: mol_type = other DNA
gctttctttccagaaggttcc

SEQ ID NO: 28 - DNA, 19 bp, synthetic construct
  misc_feature 1..19: note = Caskin1 E14/17 forward primer
  source 1..19: mol_type = other DNA
caccgaagaagctggaatc

SEQ ID NO: 29 - DNA, 19 bp, synthetic construct
  misc_feature 1..19: note = Caskin1 E14/17 reverse primer
  source 1..19: mol_type = other DNA
ccaggtgatatcggtgatg

SEQ ID NO: 30 - DNA, 22 bp, synthetic construct
  misc_feature 1..22: note = Tsn E4/6 forward primer
  source 1..22: mol_type = other DNA
gtttcatgagcattggcggttc

SEQ ID NO: 31 - DNA, 22 bp, synthetic construct
  misc_feature 1..22: note = Tsn E4/6 reverse primer
  source 1..22: mol_type = other DNA
gtagtctccagcagtgacactg

SEQ ID NO: 32 - DNA, 21 bp, synthetic construct
  misc_feature 1..21: note = GAPDH E3/5 forward primer
  source 1..21: mol_type = other DNA
accaccatggagaaggccggg

SEQ ID NO: 33 - DNA, 21 bp, synthetic construct
  misc_feature 1..21: note = GAPDH E3/5 reverse primer
  source 1..21: mol_type = other DNA
cagtgatggcatggactgtgg

SEQ ID NO: 34 - DNA, 1032 bp, Escherichia phage P1
  source 1..1032: mol_type = genomic DNA
atggtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggac

(19) United States
(12) Patent Application Publication
(10) Pub. No.: US 2025/0344681 A1
(43) Pub. Date: Nov. 13, 2025

(54) ANIMAL MODEL OF TDP-43 PROTEINOPATHY

(71) Applicant: Regeneron Pharmaceuticals, Inc., Tarrytown, NY (US)

(72) Inventors: Sharma-Kanning Aarti, Croton-on-Hudson, NY (US); Tisdale Sarah, Stamford, CT (US); Frendewey David, New York, NY (US)

(21) Appl. No.: 18/867,765

(22) PCT Filed: May 26, 2023
(86) PCT No.: PCT/US2023/067523

§ 371 Date: Nov. 20, 2024

Related U.S. Application Data
(60) Provisional application No. 63/347,262, filed on May 31, 2022.

(51) Int. Cl. A01K 67/0276; A01K 67/0278; C07K 14/47; C12N 9/12; C12N 15/86

(52) U.S. Cl. CPC A01K 67/0276; A01K 67/0278; C07K 14/4702; C12N 9/1241; C12N 15/86; A01K 2217/075; A01K 2217/206; A01K 2227/105; A01K 2267/0318; C07K 2319/09; C12N 2750/14143; C12N 2800/30

(57) ABSTRACT

Described herein is the discovery that TDP-43 proteinopathies may be induced in adult or neonatal animals bearing on one chromosome a mutant TARDBP gene encoding a mutant TDP-43 protein that lacks a functional nuclear localization signal (NLS) or a mutant TARDBP gene encoding a mutant TDP-43 protein that lacks a prion-like domain (PLD) and on the other homologous chromosome a TARDBP gene comprising a conditional knockout mutation. Knockout of the TARDBP gene comprising the conditional knockout mutation, e.g., using Cre recombinase, during the neonatal stage, e.g., at P0-P10, or during adulthood, e.g., at about 5 months of age, results in the mice exhibiting neuromuscular phenotypes such as early lethality, paralysis, weight loss, etc. These animals exhibit hallmark symptoms of ALS and that may be used in testing candidate agents useful in treating TDP-43 proteinopathies.

### CROSS-REFERENCE TO RELATED APPLICATIONS

This application claims the benefit under 35 U.S.C. § 119(3) of U.S. Provisional Application Ser. No. 63/347,262 (filed May 31, 2022), the disclosure of which is hereby incorporated by reference in its entirety.

### SEQUENCE LISTING SUBMITTED AS A TEXT FILE VIA EFS WEB

The official copy of the sequence listing is submitted electronically via EFS-Web as an ASCII formatted sequence listing with a file named 11233WO01_ST26, created on May 25, 2023, and having a size of 70 kilobytes, and is filed concurrently with the specification. The sequence listing contained in this ASCII formatted document is part of the specification and is herein incorporated by reference in its entirety.

### TECHNICAL FIELD

Described herein are non-human animal model of TDP-43 proteinopathies comprising a non-human animal in which cells in its central nervous system express only a mutant TDP-43 protein and do not express a wildtype TDP-43 protein, and methods of making and using same.

### BACKGROUND OF THE INVENTION

Amyotrophic lateral sclerosis (ALS) is a devastating neurodegenerative disease that affects motor neurons, causing limb paralysis and eventual death as the result of failure of the diaphragm muscle. A nearly universal pathological finding in postmortem examinations of ALS patient tissue is the accumulation of TDP-43 (transactive response DNA binding protein 43 kDa) in cytoplasmic inclusions.

TDP-43 is a predominantly nuclear RNA binding protein similar in structure to members of the heterogeneous nuclear ribonucleoprotein (hnRNP) family. Several structural features, e.g., domains, of the TDP-43 protein have been identified, including a nuclear localization signal (NLS), two RNA recognition motifs (RRM1 and RRM2), a putative nuclear export signal (NES), and a large domain in the carboxyl-terminal half of the protein that has been described as a low complexity, poorly ordered, or prion-like domain (PLD). Similar to members of the hnRNP family, TDP-43 is a predominantly nuclear RNA binding protein required for the viability of all mammalian cells and the normal development of animals. The biological function of TDP-43 has yet to be fully elucidated, but there is evidence that the protein participates in the regulation of pre-messenger RNA (pre-mRNA) splicing by preventing the use of cryptic exons in large introns and by influencing alternative splicing of several pre-mRNAs. TDP-43 is also proposed to have functions in the cytoplasm, perhaps in the shuttling of RNAs between the nucleus and cytoplasm and in the transport of mRNAs within the axons of neurons. Of the mutations in TDP-43 that are associated with familial cases of ALS, most are found in the PLD. The redistribution of TDP-43 from the nucleus to the cytoplasm and its accumulation in insoluble aggregates are two key diagnostic hallmarks of ALS disease.

Although TDP-43 appears to be involved in the ALS onset and/or progression, there is a need for animal models of TDP-43 proteinopathy to help understand the role of TDP-43 in ALS pathogenesis.

### SUMMARY OF THE INVENTION

Described herein are non-human animals (e.g., rodents (e.g., rats or mice)) that exhibit TDP-43 proteinopathies and associated ALS-like symptoms when the non-human animal is forced to express only a mutant form of TDP-43 that lacks a functional TDP-43 nuclear localization signal or that lacks a functional TDP-43 prion-like domain, e.g., in the CNS. Compositions and methods of making such non-human animals, and methods of using the non-human animal are also provided.

In some embodiments, a non-human animal as described herein comprises, in its central nervous system (CNS), a plurality of cells that each comprises: (a) a mutated TARDBP gene at one chromosome at an endogenous TARDBP locus and (b) a knockout TARDBP gene at the other homologous chromosome at an endogenous TARDBP locus, wherein the knockout TARDBP gene comprises the wildtype TARDBP gene sequence that comprises a loss of-function mutation. In some embodiments, the mutated TARDBP gene comprises a wildtype TARDBP gene sequence (e.g., a wildtype endogenous TARDBP gene of the non-human animal or a wildtype TARDBP gene) that comprises a mutation in a nuclear localization signal (NLS) encoding sequence or a prion like domain (PLD) encoding sequence such that the mutated TARDBP gene encodes a mutant TDP-43 polypeptide that lacks a functional TDP-43 nuclear localization signal (NLS) or lacks a functional prion like domain (PLD), In some embodiments, the knockout TARDBP gene comprises a deletion of its exon 3. In some embodiments, the plurality of cells comprises neurons. In some embodiments, the non-human animal further comprises a second plurality of cells (which second plurality of cells may comprise germ cells, and/or somatic cells other than neuron and/or glial cells), wherein each of the second plurality of cells comprises (a) the mutated TARDBP gene on one chromosome at an endogenous TARDBP locus, and (b) a conditional knockout TARDBP gene at the other homologous chromosome at an endogenous TARDBP locus, wherein the conditional knockout TARDBP gene comprises the wildtype TARDBP gene sequence with at least one exon flanked by a site-specific recombinase recognition sequence and encodes a wildtype TDP-43 protein, and wherein recognition of the site-specific recombinase recognition sequence by a recombinase results in the deletion of at least one exon and formation of the knockout TARDBP gene.

In some embodiments, exon 3 of the conditional knockout TARDBP gene is flanked by the site-specific recombinase recognition sequence, e.g., wherein the site-specific recombinase recognition sequence comprises a loxP sequence and the recombinase is Cre recombinase. In some embodiments, the non-human animal further a recombinase that recognizes the recombinase recognition sequence. For example, in some embodiments, the non-human further comprises a nucleic acid comprising a sequence that encodes a recombinase, wherein the nucleic acid further comprises (i) a promoter sequence that drives the expression of the recombinase, (ii) a reporter gene sequence, optionally wherein the reporter gene sequence is operably linked to the recombinase gene sequence by a poly A sequence, (iii) an adeno-associated virus (AAV) inverted terminal repeat (ITR) sequence at the 5′ and 3′ ends of the nucleic acid, or (iv) any combination of (i)-(iii). In some embodiments, the promoter sequence comprises a CNS-tissue specific promoter sequence, e.g., a synapsin promoter sequence, e.g., a human synapsin promoter sequence. In some embodiments, the nucleic acid comprises a sequence set forth as SEQ ID NO:18 or SEQ ID NO:19.

In some embodiments, a wildtype TARDBP gene is an endogenous wildtype TARDBP gene of the non-human animal. In some embodiments, a wildtype TARDBP gene is a wildtype human TARDBP gene.

In some embodiments, the mutant TDP-43 polypeptide comprises (a) a point mutation of an amino acid in the NLS, or (b) a deletion of at least a portion of the prion-like domain. For example, in some embodiments, the point mutation of an amino acid in the NLS comprises K82A K83A, R84A, K95A, K97A, K98A, or a combination thereof, and/or the deletion of at least a portion of the prion-like domain comprises a deletion of the amino acids at and between positions 274 and 414 of a wildtype TDP 43 polypeptide. In some embodiments, the mutant TDP-43 polypeptide comprises K82A K83A, R84A, K95A, K97A, and K98A point mutations. In some embodiments, the mutant TDP-43 polypeptide lacks the prion like domain between and including the amino acids at positions 274 to 414 of a wildtype polypeptide.

In some embodiments, the mutated TARDBP gene replaces an endogenous TARDBP gene, and the knockout TARDBP gene (and/or the TARDBP gene comprising a conditional knockout mutation) replaces an endogenous TARDBP gene.

In some embodiments, the non-human animal is a rat. In some embodiments, the non-human animal is a mouse.

In some embodiments, the non-human animal exhibits one or more of the following TDP-43 proteinopathy characteristics in comparison to a control non-human animal:
(i) weight loss
(ii) cytoplasmic aggregation of TDP-43,
(iii) decreased number of motor neurons in the spinal cord,
(iv) disruption of TDP-43 function in cryptic and alternative splicing (e.g., disruption of transcript processing of an mRNA transcript dependent on TDP-43, e.g., an mRNA transcript selected from the group Adnp2, Dnajc5, Poldip3, Tsn, and Sortilin1),
(v) denervation of neuromuscular junctions (e.g., neuromuscular junctions in tibialis anterior muscle, a gastrocnemius muscle, soleus muscle, a muscle of the bicep muscle group, tricep muscle group and/or intercostal muscles),
(vi) a motor phenotype, e.g., wherein the motor phenotype is selected from the group consisting of hind limb clasping, kyphosis, early hyperactivity, uncoordinated/ataxic movement, head wobbling, paralysis, inability to right, and a combination thereof, and/or
(vii) early lethality,
wherein each cell of the control non-human animal comprises:
(a) the mutated TARDBP gene at one chromosome at an endogenous TARDBP locus, and
(b) a wildtype TARDBP gene or a conditional knockout TARDBP gene at the other homologous chromosome at an endogenous TARDBP locus,
wherein the conditional knockout TARDBP gene comprises the wildtype TARDBP gene sequence with at least one exon flanked by a site-specific recombinase recognition sequence and encodes a wildtype TDP 43 protein, and
wherein recognition of the site-specific recombinase recognition sequence by a recombinase results in the deletion of at least one exon and formation of the knockout TARDBP gene. In some embodiments, both the non-human animal and the control non-human animal are each a rat. In some embodiments, both the non-human animal and the control non-human animal are each a mouse.

In some embodiments, the decreased number of motor neurons in the spinal cord of a non-human animal of TDP-43 proteinopathy as described herein comprises a decrease in the number of alpha motor neurons compared to the number of alpha motor neurons in the spinal cord of a control animal expressing wildtype TDP-43. In some embodiments, the decreased number of motor neurons in the spinal cord of a non-human animal of TDP-43 proteinopathy as described herein comprises at least a 5% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 10% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 15% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 20% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 25% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 30% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 35% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 40% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 45% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 50% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises a statistically significant decrease in the number of alpha motor neurons compared to the number of alpha motor neurons in the spinal cord of a control animal expressing a wildtype TDP-43. In some embodiments, the number of gamma motor neurons in the spinal cord of a non-human animal of TDP-43 proteinopathy as described herein is not significantly different to the number of gamma motor neurons in the spinal cord of a control animal expressing a wildtype TDP-43. Thus, in some embodiments, a non-human animal model of TDP-43 proteinopathy as described herein comprises a decreased number of motor neurons in the spinal cord, wherein the decreased number of motor neurons in the spinal cord comprises a selective loss of alpha motor neurons, e.g., a decreased number of alpha motor neurons in the spinal cord of the animal model of TDP-43 proteinopathy compared to a control animal expressing a wildtype TDP-43 (e.g., at least a 5%, 10%, 15%, 20%, 25%, 30%, 35%, 40%, 45%, 50% decrease and/or a statistically significant decrease), and wherein the number of gamma motor neurons in the spinal cord of the non-human animal of TDP-43 proteinopathy is at least 96% or more of, and/or is not significantly different than, the number of gamma motor neurons in the spinal cord of the control animal.

In some embodiments of an accelerated model described herein, achieved by neural- and/or glial-specific knockout of the wildtype TARDBP gene at P0/P1 in these animals, the animals may proceed to exhibit, e.g., (i) severe motor phenotypes by, at, and/or around, 4 weeks of age, and/or (ii) disruption of TDP-43 function in cryptic and alternative splicing; and/or denervation of neuromuscular junctions in tibialis anterior, gastrocnemius, and soleus muscles, selective loss of alpha motor neurons by, at, and/or around, 10 weeks of age, and/or (iii) and early lethality by, at, or around, 7-12 weeks of age.

In some non-human animal cell embodiments, the non-human animal cell is isolated from a non-human animal a described herein. In some embodiments, the non-human animal cell comprises:
(a) the mutated TARDBP gene at one chromosome at an endogenous TARDBP locus, and
(b) the knockout TARDBP gene at the other homologous chromosome at an endogenous TARDBP locus. In some non-human animal cell embodiments, the non-human animal cell is part of a composition, e.g., a composition cultured in vitro comprising the non-human animal cell and appropriate culture media.

Also described herein is a method of identifying a therapeutic candidate for the treatment of TDP-proteinopathy and/or an associated disease (e.g., ALS). In some embodiments, a method of identifying a therapeutic candidate agent for the treatment of TDP-proteinopathy and/or associated diseases comprises (a) contacting a non-human animal comprising a knockout TARDBP gene as described herein with the candidate agent, (b) evaluating a phenotype and/or a biological function of TDP-43 in the non-human animal, and (c) identifying the candidate agent that prevents or reduces the exhibition of one or more of the following TDP-43 proteinopathy characteristics in the non-human animal:
(i) weight loss
(ii) cytoplasmic aggregation of TDP-43 (e.g., wherein the candidate agent restores nuclear localization of TDP-43),
(iii) decreased number of motor neurons in the spinal cord,
(iv) disruption of TDP-43 function in cryptic and alternative splicing (e.g., disruption of transcript processing of an mRNA transcript dependent on TDP-43, e.g., an mRNA transcript selected from the group Adnp2, Dnajc5, Poldip3, Tsn, and Sortilin1),
(v) denervation of neuromuscular junctions, e.g., in tibialis anterior muscle, gastrocnemius muscle, a soleus muscle, a muscle of the bicep muscle group, a muscle of the tricep muscle group and/or an intercostal muscle,
(vi) a motor phenotype, such as but not limited to a motor phenotype selected from the group consisting of hind limb clasping, early hyperactivity, uncoordinated/ataxic movement, head wobbling, paralysis, inability to right, and a combination thereof, and/or
(vii) early lethality.

In some embodiments, a method of making a non-human animal model of TDP-43 proteinopathy comprises:
(I) modifying the genome of a non-human animal to comprise:
   (a) a mutated TARDBP gene on one chromosome at an endogenous TARDBP locus, and
   (b) a conditional knockout TARDBP gene at the other homologous chromosome at an endogenous TARDBP locus,
   wherein the conditional knockout TARDBP gene comprises the wildtype TARDBP gene sequence with at least one exon flanked by a site-specific recombinase recognition sequence and encodes a wildtype TDP-43 protein; and
(II) administering to the non-human animal a recombinase that recognizes the site-specific recombinase recognition sequence to create the knockout TARDBP gene from the conditional knockout TARDBP gene, wherein contacting the site-specific recombinase recognition sequence by a recombinase results in the deletion of at least one exon and formation of a knockout TARDBP gene
wherein after the administering step, the non-human animal exhibits one or more TDP-43 proteinopathy characteristics in comparison to a control non-human animal. In some embodiments, one or more TDP-43 proteinopathy characteristics comprises:
(i) weight loss(ii) cytoplasmic aggregation of TDP-43,
(iii) decreased number of motor neurons in the spinal cord,
(iv) disruption of TDP-43 function in cryptic and alternative splicing (e.g., disruption of transcript processing of an mRNA transcript dependent on TDP-43, e.g., an mRNA transcript selected from the group Adnp2, Dnajc5, Poldip3, Tsn, and Sortilin1),
(v) denervation of neuromuscular junctions (e.g., neuromuscular junctions in tibialis anterior muscle, a gastrocnemius muscle, soleus muscle, a muscle of the bicep muscle group, tricep muscle group and/or intercostal muscles),
(vi) a motor phenotype, e.g., wherein the motor phenotype is selected from the group consisting of hind limb clasping, early hyperactivity, uncoordinated/ataxic movement, head wobbling, paralysis, inability to right, and a combination thereof, and/or
(vii) early lethality.
   In some embodiments, the decreased number of motor neurons in the spinal cord of a non-human animal of TDP-43 proteinopathy as described herein comprises a decrease in the number of alpha motor neurons compared to the number of alpha motor neurons in the spinal cord of a control animal expressing wildtype TDP-43. In some embodiments, the decreased number of motor neurons in the spinal cord of a non-human animal of TDP-43 proteinopathy as described herein comprises at least a 5% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 10% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 15% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 20% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 25% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 30% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 35% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 40% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 45% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises at least a 50% decrease in the number of alpha motor neurons compared to the number of alpha motor neurons in the spinal cord of a control animal expressing a wildtype TDP-43. In some embodiments, the decreased number of motor neurons in the spinal cord of a non-human animal of TDP-43 proteinopathy as described herein comprises a statistically significant decrease in the number of alpha motor neurons compared to the number of alpha motor neurons in the spinal cord of a control animal expressing a wildtype TDP-43. In some embodiments, the number of gamma motor neurons in the spinal cord of a non-human animal of TDP-43 proteinopathy as described herein is not significantly different to the number of gamma motor neurons in the spinal cord of a control animal expressing a wildtype TDP-43. Thus, in some embodiments, a non-human animal model of TDP-43 proteinopathy as described herein comprises a decreased number of motor neurons in the spinal cord, wherein the decreased number of motor neurons in the spinal cord comprises a selective loss of alpha motor neurons, e.g., a decreased number of alpha motor neurons in the spinal cord of the animal model of TDP-43 proteinopathy compared to a control animal expressing a wildtype TDP-43 (e.g., at least a 5%, 10%, 15%, 20%, 25%, 30%, 35%, 40%, 45%, 50% decrease and/or a statistically significant decrease), and wherein the number of gamma motor neurons in the spinal cord of the non-human animal of TDP-43 proteinopathy is at least 96% or more of, and/or is not significantly different than, the number of gamma motor neurons in the spinal cord of the control animal. In some embodiments, each cell of the control non-human animal comprises: (a) the mutated TARDBP gene at one chromosome at an endogenous TARDBP locus, and (b) a wildtype TARDBP gene or the conditional knockout TARDBP gene at the other homologous chromosome at an endogenous TARDBP locus.

In some embodiments, the step of modifying the genome of a non-human animal comprises:
(i) modifying the genome of the non-human animal embryonic stem (ES) cell to comprise:
   (a) a mutated TARDBP gene on one chromosome at an endogenous TARDBP locus,
   wherein the mutated TARDBP gene comprises a wildtype TARDBP gene sequence that comprises a mutation in a nuclear localization signal (NLS) encoding sequence or a prion like domain (PLD) encoding sequence such that the mutated TARDBP gene encodes a mutant TDP 43 polypeptide that lacks a functional nuclear localization signal (NLS) or a prion like domain (PLD), and
   (b) a conditional knockout TARDBP gene at the other homologous chromosome at an endogenous TARDBP locus,
   wherein the conditional knockout TARDBP gene comprises the wildtype TARDBP gene sequence with at least one exon flanked by a site-specific recombinase recognition sequence and encodes a wildtype TDP 43 protein,
   wherein recognition of the site-specific recombinase recognition sequence by a recombinase results in a deletion of the at least one exon and formation of a knockout TARDBP gene, and
   wherein the knockout TARDBP gene comprises the wildtype TARDBP gene sequence that comprises a loss of-function mutation resulting from the deletion of the at least one exon of the knockout TARDBP gene;
(ii) introducing the modified ES cell into a host non-human animal embryo; and
(iii) gestating the host non-human animal embryo in a surrogate non-human animal mother, wherein the surrogate mother births non-human animal progeny comprising:
   (a) the mutated TARDBP gene on one chromosome at an endogenous TARDBP locus, and
   (b) the conditional knockout TARDBP gene on the other homologous chromosome at an endogenous TARDBP locus.

In some embodiments, the step of administering comprises administering to the non-human animal progeny the recombinase that recognizes the site-specific recombinase recognition sequence to create a knockout TARDBP gene from the conditional knockout TARDBP gene, wherein the non-human animal progeny exhibits one or more TDP-43 proteinopathy characteristics in comparison to a control non-human animal,
wherein the one or more TDP-43 proteinopathy characteristics comprises:
   (i) weight loss
   (ii) cytoplasmic aggregation of TDP-43,
   (iii) decreased number of motor neurons in the spinal cord,
   (iv) disruption of TDP-43 function in cryptic and alternative splicing (e.g., disruption of transcript processing of an mRNA transcript dependent on TDP-43, e.g., an mRNA transcript selected from the group Adnp2, Dnajc5, Poldip3, Tsn, and Sortilin1),
   (v) denervation of neuromuscular junctions (e.g., neuromuscular junctions in tibialis anterior muscle, a gastrocnemius muscle, soleus muscle, a muscle of the bicep muscle group, tricep muscle group and/or intercostal muscles),
   (vi) a motor phenotype, e.g., wherein the motor phenotype is selected from the group consisting of hind limb clasping, early hyperactivity, uncoordinated/ataxic movement, head wobbling, paralysis, inability to right, and a combination thereof, and/or
   (vii) early lethality, and
wherein each cell of the control non-human animal comprises:
   (a) the mutated TARDBP gene at one chromosome at an endogenous TARDBP locus, and
   (b) a wildtype TARDBP gene or the conditional knockout TARDBP gene at the other homologous chromosome at an endogenous TARDBP locus.

In some methods of making a non-human animal as described herein, the step of administering does not occur during embryogenesis. In some methods of making a non-human animal as described herein, the step of administering takes place neonatally, e.g., at P0-P10 after birth of the non-human animal, e.g., the non-human animal progeny. In some embodiments, the non-human animal exhibits the one or more TDP-43 proteinopathy characteristics by, around, and/or in as little as, four to five weeks after the administering step. In some embodiments, non-human animal exhibits at least two of the one or more TDP-43 proteinopathy characteristics by, at and/or around about seven to ten weeks after the administering step. In some methods of making a non-human animal as described herein, the step of administering takes place 1 month, 2 months, 3 months, 4 months, 5 months, or 6 months after birth of the non-human animal progeny, and the non-human animal progeny exhibits the one or more one or more TDP-43 proteinopathy characteristics 5-7 months after the administering step. In some methods of making a non-human animal as described herein, exon 3 of the conditional knockout TARDBP gene is flanked by the site-specific recombinase recognition sequence. In some methods of making a non-human animal as described herein, the site-specific recombinase recognition sequence comprises a loxP sequence and the recombinase is Cre recombinase. In some methods of making a non-human animal as described herein, the administering step comprises intraperitoneal or intracerebroventricular injection of a nucleic acid comprising a sequence that encodes the recombinase. In some methods of making a non-human animal as described herein, the administering step comprises intraperitoneal or intracerebroventricular injection of AAV particles (e.g., AAV-PHP.eB particles) comprising a nucleic acid comprising a sequence that encodes the recombinase, wherein the nucleic acid further comprises:
(i) a promoter sequence that drives the expression of the recombinase,
(ii) optionally, a reporter gene sequence, further optionally wherein the reporter gene sequence is operably linked to the recombinase gene sequence by a poly A sequence,
(iii) an adeno-associated virus (AAV) inverted terminal repeat (ITR) sequence at the 5′ and 3′ ends of the nucleic acid,
(iv) any combination of (i)-(iii).

In some methods of making a non-human animal as described herein, the nucleic acid comprising a sequence that encodes the recombinase comprises the sequence set forth as SEQ ID NO:18 or SEQ ID NO:19. In some methods of making a non-human animal as described herein, the conditional knockout TARDBP gene comprises the wildtype TARDBP gene comprising a site-specific recombinase recognition sequence that flanks its exon 3. In some methods of making a non-human animal as described herein, the wildtype TARDBP gene is an endogenous wildtype TARDBP gene of the non-human animal. In some methods of making a non-human animal as described herein, the wildtype TARDBP gene is a wildtype human TARDBP gene. In some methods of making a non-human animal as described herein, the mutant TDP 43 polypeptide comprises (a) a point mutation of an amino acid in the NLS, and/or (b) a deletion of at least a portion of the prion-like domain. In some methods of making a non-human animal as described herein, (a) the point mutation of an amino acid in the NLS comprises K82A K83A, R84A, K95A, K97A, K98A, or a combination thereof, and/or (b) the deletion of at least a portion of the prion-like domain comprises a deletion of the amino acids at and between positions 274 and 414 of a wildtype TDP 43 polypeptide. In some methods of making a non-human animal as described herein, the mutant TDP-43 polypeptide comprises K82A K83A, R84A, K95A, K97A, and K98A point mutations. In some methods of making a non-human animal as described herein, the mutant TDP-43 polypeptide lacks the prion like domain between and including the amino acids at positions 274 to 414 of a wildtype polypeptide. In some methods of making a non-human animal as described herein, modifying comprises replacing an endogenous TARDBP gene on one chromosome with the mutated TARDBP gene, and replacing an endogenous TARDBP gene at the other homologous chromosome with the conditional knockout TARDBP gene. In some methods of making a non-human animal as described herein, the non-human animal is a rat. In some methods of making a non-human animal as described herein, the non-human animal is a mouse.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides an illustration (not to scale) of TDP-43, the relative position for the nuclear localization signal (NLS; amino acids 82-98), the relative positions for the two RNA recognition motifs (RRM1; amino acids 106-176, and RRM2; amino acids 191-262), the relative position for a putative nuclear export signal (E; amino acids 239-248), the relative position for a prion like domain (PLD; amino acids 274-414), ALS-associated amino acid substitution mutations, and ALS-associated C terminal fragments. Asterisks highlight mutations associated with FTD symptoms with or without ALS. A90V, S92L, N267S, G287S, G294V, G368S, S375G, A382T, I383V, N390S, and N390D mutations have also been observed in healthy individuals.

FIG. 2A provides an illustration (not to scale) of the mouse TARDBP genomic structure, which depicts exons 1-6 (rectangles), untranslated regions (unfilled rectangles), and translated regions (filled rectangles) starting with the ATG start codon. FIG. 2B provides an amino acid sequence alignment of mouse (m) TDP-43 and human (h) TDP-43 polypeptides, the amino acid positions of the polypeptides, and a consensus sequence underneath the mTDP-43 and hTDP-43 sequences. Generally, boxed regions within the alignment show the nuclear localization signal (NLS: amino acids 82-98), RNA recognition motif 1 (RRM1: amino acids 106-176). RNA recognition motif 2 (RRM2: amino acids 191-262), a putative nuclear export signal (E: amino acids 239-248), and the glycine rich prion-like domain (PLD: amino acids 274-414). Amino acid mismatches between mouse TDP-43 and human TDP-43 are also boxed and depicted by a dash in the consensus sequence. Exon junctions are also depicted as vertical lines denoting the exons (EX) joined at the denoted junction. The vertical line between amino acids 286 and 287 provides an alternative 5′-splice site.

FIG. 3A provides illustrations (not-to-scale) of (1) an exemplary TARDBP gene that comprises a conditional knockout mutation and encodes a wildtype TDP-43 protein, wherein the conditional knockout mutation comprises exon 3 flanked by loxP site-specific recombination recognition sites (triangles), hereinafter referred to as “loxP-Ex3-loxP” or “flEx3” in the absence of the condition and as “ΔEx3” after removal of exon 3 in the presence of the condition, e.g., upon Cre-mediated recombination; and (2) a TARDBP null allele comprising a deletion of the entire TARDBP coding sequence hereinafter referred to as “ACDS” or “-”. Depicted are exons 1-6 (rectangles), untranslated regions (unfilled rectangles), translated regions (filled rectangles), and relative locations of the start ATG and stop TGA codons. FIG. 3B provides illustrative depictions (not-to-scale) of non-limiting mutant TDP-43 polypeptides encoded by various forms of mutated TARDBP genes. Throughout these Examples and associated Figures:
“WT” refers to a wildtype TARDBP gene,
“-” refers to a TARDBP gene lacking the entire TDP-43 coding sequence,
“loxP-Ex3-loxP” or “flEx3” refers to a mutated TARDBP gene comprising a conditional knockout mutation that, in the absence of the condition (i.e., Crc recombinase) encodes a wildtype TDP-43 protein, wherein the conditional knockout mutation comprises a floxed exon 3,
“ΔEx3” refers to a mutated TARDBP gene lacking a nucleotide sequence comprising the sequence of exon 3 of a wildtype TARDBP gene upon Cre-mediated recombination of loxP-Ex3-loxP,
“ΔNLS” refers to a mutated TARDBP gene that encodes a mutant TDP-43 polypeptide comprising the following point mutations: K82A, K83A, R84A, K95A, K97A, and K98A,
“ΔPLD” refers to a mutated TARDBP gene that encodes a mutant TDP-43 polypeptide lacking amino acids 274 to 414 of a wildtype TDP-43 polypeptide.
   For the ΔPLD mutant TDP-43 polypeptides, diagonal lines represent regions that are deleted.

FIG. 4 provides a graph showing the survival time post fertilization of 8-cell embryos injected with TDP-43^{ΔEx3/-} ES cells, TDP-43^{ΔNLS/ΔEx3} modified ES cells, TDP-43^{ΔPLD/ΔEx3} modified ES cells, wildtype TDP-43^{WT/WT} ES cells, TDP-43^{WT/-} modified ES cells, TDP-43^{flEx3/flEx3} modified ES cells, TDP-43^{ΔNLS/WT} modified ES cells, TDP-43^{ΔNLS/flEx3} modified ES cells, TDP-43^{ΔPLD/WT} modified ES cells, or TDP-43^{ΔPLD/flEx3} modified ES cells. E3.5 (embryonic day 3.5), E 10.5 (embryonic day 10.5), E 15.5 (embryonic day 15.5), P0 (postnatal day 0).

FIG. 5 provides a graph showing the percentage of mice that survived (y-axis) for at least 15 months (x-axis) before and after Cre mediated deletion of exon 3 of a TARDBP gene comprising the floxed exon 3 conditional knockout mutation. The following are the genotypes of the mice studied after Cre mediated deletion of the floxed exon 3: TDP-43^{WT/WT}, TDP-43^{ΔEx3/WT}, TDP-43^{ΔNLS/ΔEx3}, and TDP-43^{ΔPLD/ΔEX3}. In this study, twelve animals per group with injected intraperitoneally (i.p.) at 5 months of age with 1×10¹¹ viral AAV-PHP.eB-hSyn-Cre genomes. The following are the genotypes of the mice studied after Cre mediated deletion of the floxed exon 3: TDP-43^{WT/WT}, TDP-43^{ΔEx3/WT}, TDP-43^{ΔNLS/ΔEx3}, and TDP-43^{ΔPLD/ΔEX3}.

FIG. 6 provides a graph showing the weight (y-axis; grams) over time (x-axis) of mice after Cre mediated deletion of exon 3 of a TARDBP gene comprising the floxed exon 3 conditional knockout mutation. The following are the genotypes of the mice studied after Cre mediated deletion of the floxed exon 3: TDP-43^{WT/WT} (▾) TDP-43^{ΔEx3/WT} (▪) TDP-43^{ΔNLS/ΔEx3} (▴), TDP-43^{ΔPLD/ΔEX3} (●). In this study, twelve animals per group with injected intraperitoncally (i.p.) at 5 months of age with 1×10¹¹ viral AAV-PHP.eB-hSyn-Cre genomes. The following are the genotypes of the mice studied after Cre mediated deletion of the floxed exon 3: TDP-43^{WT/WT}, TDP-43^{ΔEx3/WT}, TDP-43^{ΔNLS/ΔEx3}, TDP-43^{ΔPLD/ΔEX3}.

FIG. 7 provides fluorescence immunohistochemistry images at 40× magnification of motor neurons isolated from spinal cord tissue isolated from 12-13 month old mice (end stage), 7-8 months after Cre mediated deletion of exon 3 of a TARDBP gene comprising the floxed exon 3 conditional knockout mutation. In this study, twelve animals per group with injected intraperitoneally (i.p.) at 5 months of age with 1×10¹¹ viral AAV-PHP.eB-hSyn-Cre genomes. The following are the genotypes of the mice studied after Cre mediated deletion of the floxed exon 3: TDP-43^{WT/WT}, TDP-43^{ΔEx3/WT}, TDP-43^{ΔNLS/ΔEx3}, TDP-43^{ΔPLD/ΔEX3}.

FIG. 8A provides the percentage of ChAT⁺ motor neurons (MNs) in lumbar segments 4-6 (L4-L6) of mice sacrificed at 12-13 months of age (end stage) and 7-8 months after Cre mediated deletion of exon 3 of a TARDBP gene comprising the floxed exon 3 conditional knockout mutation. FIG. 8B provides the percent denervation in tibialis anterior muscle tissue of mice sacrificed at 12-13 months of age (end stage) and 7-8 months after Cre mediated deletion of exon 3 of a TARDBP gene comprising the floxed exon 3 conditional knockout mutation. FIG. 8C provides the percent denervation in soleus muscle tissue of mice sacrificed at 12-13 months of age (end stage) and 7-8 months after Cre mediated deletion of exon 3 of a TARDBP gene comprising the floxed exon 3 conditional knockout mutation. In this study, twelve animals per group with injected intraperitoneally (i.p.) at 5 months of age with 1×10¹¹ viral AAV-PHP.eB-hSyn-Cre genomes. The following are the genotypes of the mice studied after Cre mediated deletion of the floxed exon 3: TDP-43^{WT/WT}, TDP-43^{ΔEx3/WT}, TDP-43^{ΔNLS/ΔEx3}, TDP-43^{ΔPLD/ΔEX3}.

FIG. 9A provides a graph showing the percentage of surviving mice (y-axis) at 52 weeks (x-axis) after CAG-Cre mediated deletion of exon 3 of a TARDBP gene comprising the floxed exon 3 conditional knockout mutation. In this study, either 3.5×10¹⁰ or 5×10¹⁰ viral AAV-PHP.eB-CAG-Cre genomes were administered to P0 newborn pub mice by intracerebroventricular (i.e.v) injection. The following are the genotypes of the mice studied after CAG-Cre mediated deletion of the floxed exon 3: TDP-43^{ΔEx3/ΔEx3} (n=18), TDP-43^{ΔEx3/ΔNLS} (n=3), TDP-43^{ΔEx3/ΔPLD} (n=7), and TDP-43^{ΔEx3/WT} (n=7). Median survival times were as follows: TDP-43^{ΔEx3/ΔEx3}: 4 weeks; TDP-43^{ΔEx3/ΔNLS}: 10.86 weeks; TDP-43^{ΔEx3/ΔPLD}: 9.43 weeks; TDP-43^{ΔEx3/WT}: 17.36 weeks. FIG. 9B provides a graph showing the percentage of surviving mice (y-axis) at 52 weeks (x-axis) after SYN-Cre mediated deletion of exon 3 of a TARDBP gene comprising the floxed exon 3 conditional knockout mutation. In this study, either 3.5×10¹⁰ or 5×10¹⁰ viral AAV-PHP.eB-SYN-Cre genomes were administered to P0 newborn pub mice by i.e.v injection. The following are the genotypes of the mice studied after SYN-Cre mediated deletion of the floxed exon 3: TDP-43^{ΔEx3/ΔEx3} (n=12), TDP-43^{ΔEx3/ΔNLS} (n=1), TDP-43^{ΔEx3/ΔPLD} (n=7), and TDP-43^{ΔEx3/WT} (n=9). Median survival times were as follows: TDP-43^{ΔEx3/ΔEx3}: 3.93 weeks; TDP-43^{ΔEx3/ΔNLS}: 10.7 weeks; TDP-43^{ΔEx3/ΔPLD}: 25 weeks; TDP-43^{ΔEx3/WT}: 52 weeks. Animals that reached 52 weeks of age were sacrificed for cellular analysis.

FIG. 10A provides a graphical representation of the percent of mice that display a hindlimb clasping phenotype across time after CAG-Cre mediated deletion of exon 3 of a TARDBP gene comprising the floxed exon 3 conditional knockout mutation. FIG. 10B and FIG. 10C incorporate the early symptomatic and late symptomatic timepoints into a reproduction of the first 18 week periods for FIG. 9A and FIG. 9B, respectively. See FIG. 9A and FIG. 9B for details on experimental methods.

FIG. 11 provides graphs showing the denervation of certain skeletal muscles in mice after CAG-Cre mediated deletion of exon 3 of a TARDBP gene comprising the floxed exon 3 conditional knockout mutation. In this study, 5×10¹⁰ viral AAV-PHP.cB-CAG-Cre genomes were administered to P0 newborn pub mice by intracerebroventricular (i.e.v) injection. The following are the genotypes of the mice studied after Cre mediated deletion of the floxed exon 3: TDP-43^{ΔNLS/ΔEx3} (n=2), TDP-43^{ΔPLD/ΔEx3} (n=2). Controls included mice receiving no viral genomes with the following genotypes: TDP-43^{ΔPLD/flEx3} at 6 weeks and TDP-43^{flEx3/flEx3} at 10 weeks.

FIG. 12 provides a quantification of the average number of motor neurons in the lumbar L4-L6 spinal cord segments across genotypes. Motor neurons are distinguished by the expression of ChAT, while the alpha and gamma subtypes are distinguished by the absence (gamma) or presence (alpha) of the neuronal marker NeuN (left panel) and provides representative immunohistochemisty staining of lateral motor column motor neurons in the L4-L6 spinal cord segments from control (TDP-43^{flEx3/flEx3}, uninjected) or mutant (TDP-43Δ^{Ex3/ΔNLS}, +CAG-Cre) mice (right panel).

FIG. 13A provides a quantification of denervation of the tibialis anterior muscle in 10 week old mice of the indicated genotypes injected with SYN-Cre. FIG. 13B provides quantification of the average number of motor neurons in the lumbar L4-L6 spinal cord segments across genotypes injected with either CAG-Cre or SYN-Cre, as indicated. Motor neurons are distinguished by the expression of ChAT, while the alpha and gamma subtypes are distinguished by the absence (gamma) or presence (alpha) of the neuronal marker NeuN.

FIG. 14 is a schematic representation (not-to-scale) of TDP-43's function in RNA splicing. TDP-43 most prominently binds introns through the recognition of GU-rich sequences. The binding of TDP-43 to intronic sequences acts predominantly to suppress recognition of cryptic exons by the splicing machinery, however it can also act as a regulator of alternative splicing to either inhibit or enhance the inclusion of alternative exons.

FIG. 15 provides RT-PCR analysis of the indicated TDP-43-dependent splicing events. The specific splicing event being monitored is indicated in the mRNA schematics on the right, and the primer locations are indicated with the black arrows. Cryptic exons are indicated as red boxes in the schematics. Both PLD and NLS mutants display a clear loss of function in splicing regulation in the spinal cord of the indicated treated animals. Adnp2 and Dnajc5 assays are monitoring the aberrant inclusion of a cryptic exon, while Poldip3 and Tsn are monitoring alternative exon skipping and Sortilin1 is monitoring alternative exon inclusion.

### DETAILED DESCRIPTION

### Overview

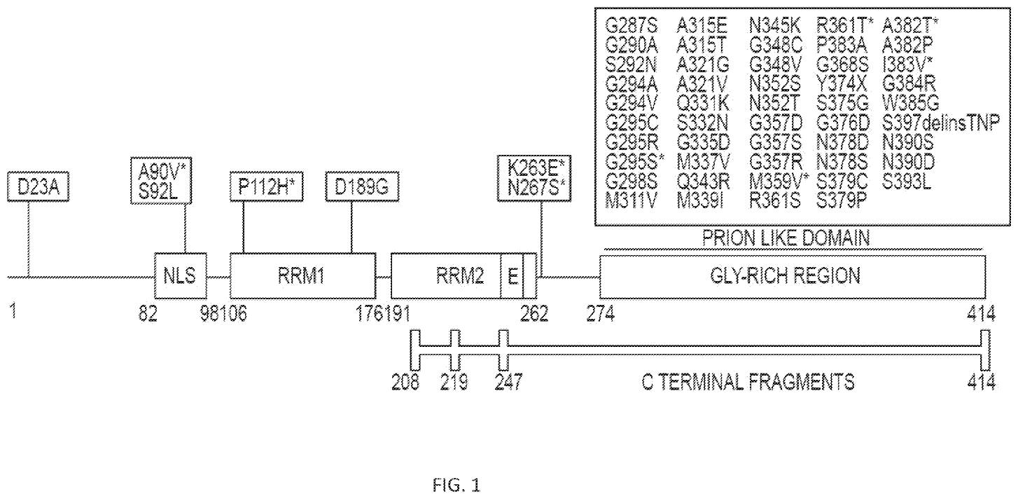

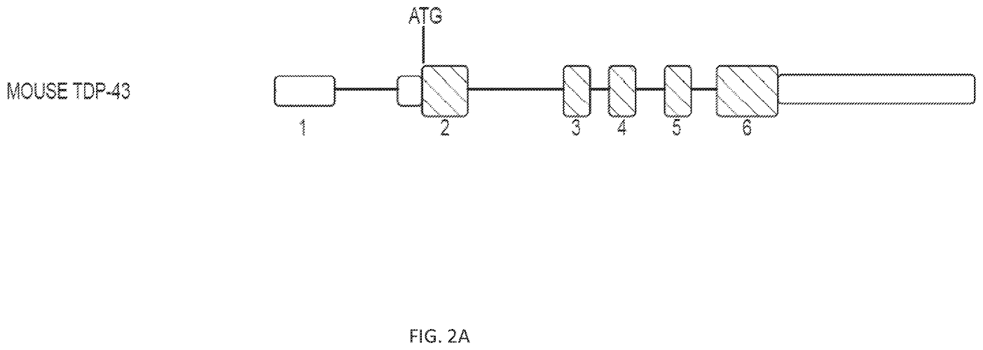

TDP-43 is a predominantly nuclear RNA/DNA-binding protein that is required for the viability of all mammalian cells and the normal development and life of animals that functions in RNA processing and metabolism, including RNA transcription, splicing, transport, and stability. The RNA-binding properties of TDP-43 appear essential for its autoregulatory activity, mediated through binding to 3′ UTR sequences in its own mRNA. Ayala et al. (2011) EMBO J. 30:277-88. Following cell stress, TDP-43 localizes to cytoplasmic stress granules and may play a role in stress granule formation. TDP-43 mislocalizes from its normal location in the nucleus to the cytoplasm, where it aggregates. Aggregated TDP-43 is ubiquinated, hyperphosphorylated, and truncated. Additionally, TDP-43 aggregation in the cytoplasm is a component of nearly all cases of ALS. Becker et al. (2017) Nature 544:367-371. Ninety-seven percent of ALS cases show a post-mortem pathology of cytoplasmic TDP-43 aggregates. The same pathology is seen in approximately 45% of sporadic Frontotemporal Lobar Degeneration (FTLDU). TDP-43 was first identified as the major pathologic protein of ubiquitin-positive, tau-negative inclusions of FTLDU, FTLD with motor neuron disease (FTDMND), and ALS/MND (ALS10), which disorders are now considered to represent different clinical manifestations of TDP-43 proteinopathy. Gitcho et al. (2009) Acta Neuropath 118:633-645. TARDBPB mutations occur in about 3% of patients with familial ALS and in about 1.5% of patients with sporadic disease. Lattante et al. (2013) Hum. Mutat. 34:812-26. Various mutations in the TARDBP gene have been associated with ALS in less than 1% of the cases. See FIG. 1. As shown in FIG. 1, the majority mutations in the TARDBP gene associated with ALS is found in the prion like domain (PLD). Therefore, understanding all the functions played by TDP-43 would likely elucidate its role in neuropathologies such as ALS, FLTDU, and FLTD, etc.

It is clear that TDP-43 is essential for cellular and organismal life. Depletion of TDP-43 results in embryonic lethality. Accordingly, initial models relied on the overexpression of TDP-43 or mutant forms thereof, or deletion of TDP-43. Various models evaluating the role of TDP-43 in ALS pathologies have been created. Reviewed in Tsao et al. (2012) Brain Res 1462:26-39.

For example, transgenic mice overexpressing a TDP-43 A315T mutant developed progressive abnormalities at about 3 to 4 months of age and died at about 5 months of age. Wegorzewska et al. (2009) Proc Nat Acad Sci USA 106:18809-814. Although the abnormalities were correlated with the presence of TDP-43 C-terminal fragments in the brain and spinal cord of these mutant mice, cytoplasmic TDP-43 aggregates were not detected. These observations led Wegorzewska et al. to suggest that neuronal vulnerability to TDP-43 associated neurodegeneration is related to altered DNA/RNA-binding protein function rather than toxic aggregation. Wegorzewska et al. (2009), supra. In contrast, in two independent studies involving the overexpression of TDP-43, transgenic mice exhibited neurodegenerative attributes including progressive motor dysfunction that was correlated with cytoplasmic aggregation. Tsai et al. (2010) J. Exp. Med. 207:1661-1673 and Wils et al (2010) Proc Natl Acad Sci USA 107:3858-63).

In loss-of function studies, ubiquitous deletion of TDP-43 using a conditional knockout mutation led to mice exhibiting a metabolic phenotype and premature death. Chiang et al. (2010) Proc Natl Acad Sci USA 107:16320-324. Depletion of TDP-43 in mouse embryonic stem cells resulted in the splicing of cryptic exons of certain genes into mRNA, disrupting translation of the mRNA and promoting nonsense-mediated mRNA decay. Ling et al. (2015) Science 349:650-655. Since postmortem brain tissue from patients with ALS/FTD show impaired repression of cryptic exon splicing, this study suggests that TDP-43 normally acts to repress the splicing of cryptic exons and maintain intron integrity, and that TDP-43 splicing defects could contribute to TDP-43-proteinopathy in certain neurodegenerative disease. Ling et al. (2015), supra. Since point mutations in the N-terminus (e.g., the NLS) of TDP-43 result in destabilization of TDP-43 oligomerization in the nucleus and loss of cryptic splicing regulation, it is hypothesized that head-to-tail oligomerization of TDP-43 driven by the N-terminus acts to separate the aggregation prone C-terminus domain (e.g., the PLD), and thus, prevent the formation of pathologic aggregates. Afroz et al. (2017) Nature Communications 8:45.

In ALS, one of the first pathological features to manifest is that the axon retracts from the neuromuscular junction causing the muscle to denervate. This denervation continues to progress resulting in the loss of the motor neuron cell body and muscle atrophy. Denervation may be observed by the loss of presynaptic markers of axon innervation: VAChT, Synaptic vesicle protein 2 (SV2), synaptophysin, and neurofilament. The motor endplate remains but will eventually fragment and disappear. Recently, dose-dependent denervation was exhibited in mice homozygous for a knockin TARDBP gene comprising disease-associated mutations. Ebstein (2019) Cell Reports 26:364-373.

Despite embryonic lethality of TDP-43 depletion, embryonic stem (ES) cells expressing a TDP-43 mutant lacking a functional domain remain viable and may be differentiated into motor neurons (ESMNs). See, WO 2020/264339A4, incorporated herein in its entirety by reference. Moreover, mutant TDP-43 polypeptides (1) lacking a functional NLS or a functional PLD and (2) at normal levels from the endogenous locus reproduces two hallmarks of ALS disease in ESMNs:
(i) redistribution of TDP-43 from the nucleus to the cytoplasm, and
(ii) accumulation in cytoplasmic inclusions. See, WO 2020/264339A4 supra.
   There was also a correlation in ESMNs between expression of a ΔPLD (e.g., deletion of the prion-like domain) or ΔNLS (e.g., knockout mutation of the nuclear localization signal) mutated TARDBP gene and a decrease in an alternative splice event involving a 3′untranslated region intron that results in an alternative spliced TDP-43 mRNA lacking sequences encoding the PLD domain, or portion thereof and the stop codon. See, WO 2020/264339A4 supra.; see also Avendano-Vazquez et al. (2012) Genes & Dev. 26:1679-84; Ayala Y M, et al. (2011) EMBO J 30: 277-288. This latter observation suggests that depleting only wildtype or ALS-associated sequences resulting from normal splice events may be potentially therapeutic for the treatment of ALS associated with PLD mutations.

Mice expressing a wildtype TARDBP gene and a ΔPLD or ΔNLS mutated TARDBP gene from endogenous loci also exhibited hallmarks of TDP-43 proteinopathies. See, WO 2020/264339A4 supra. Increased TDP-43 mislocalization from the nucleus to the cytoplasm, phosphorylation of cytoplasmic TDP-43, and cytoplasmic aggregation of TDP-43 was observed in spinal cord motor neurons of animals expressing mutant ΔPLD or ΔNLS TDP-43 polypeptides compared to animals expressing only wildtype protein. See, WO 2020/264339A4 supra. In these animals, TDP-43 mutants lacking a functional NLS, but not animals expressing TDP-43 mutants lacking a PLD, were insoluble. See, WO 2020/264339A4 supra. Moreover, denervation of muscles comprised mostly of fast twitch fibers, but not of muscles comprised mostly of slow twitch fibers, was also observed in these mice expressing mutant ΔPLD or ΔNLS TDP-43 proteins. See, WO 2020/264339A4 supra.

Although sole expression of mutant ΔPLD or ΔNLS TDP-43 proteins results in embryonic lethality, described herein is the discovery that non-human animals modified to express only mutant ΔPLD or ΔNLS TDP-43 proteins in brain tissue (e.g., neurons and/or glial cells) survive to develop markers of ALS. Neural- and/or glial-specific knockout of the wildtype TARDBP gene at 5 months after birth in animals modified to comprise at one allele a mutant gene that encodes a mutant ΔPLD or ΔNLS TDP-43 protein and at the other allele a conditional TARDBP gene that encodes a wildtype TRD-43 protein led to a twenty-five to fifty percent decline in body weight 2 months post injection accompanied by motor deficits (e.g., paralysis of the hindlimbs). An accelerated model is achieved by neural- and/or glial-specific knockout of the wildtype TARDBP gene at P0 in these animals, which animals proceed to exhibit severe motor phenotypes by, at, and/or around 4-5 weeks of age, disruption of TDP-43 function in cryptic and alternative splicing, and denervation of neuromuscular junctions in tibialis anterior, gastrocnemius, and soleus muscles, selective loss of alpha motor neurons at 10 weeks of age, and early lethality by, at, and/or around 7-12 weeks of age.

These animals are useful models to screen for genetic, chemical, and bio-molecular interventions that rescue the pathological phenotypes and might, therefore, provide ALS therapeutic leads. These models would also be valuable as tools to elucidate the biological functions and biochemical properties of TDP-43 and the proteins and RNAs with which it interacts. This basic biological information could be used to better inform strategies or discover new targets for ALS therapeutics.

### TARDBP Genes and TDP-43 Polypeptides

A TARDBP gene encodes a TDP-43 polypeptide, also referred to as TAR DNA-binding protein, TARDBP, 43-KD, and TDP43, and TDP-43. The nucleic acid sequence of wildtype TARDBP genes and the wildtype TDP-43 polypeptides encoded therefrom of different species are well known in the art. For example, the respective nucleic acid and amino acid sequences of wildtype TARDBP genes and wildtype TDP-43 polypeptides and may be found in the U.S. National Library of Medicine (NIH) National Center for Biotechnology Information (NCBI) gene database. See, e.g., the website at www.ncbi.nlm.nih.gove/gene/?term=TARDBP. In some embodiments, a wildtype mouse TARDBP gene comprises a nucleotide sequence that encodes a wildtype mouse TDP-43 polypeptide comprising an amino acid sequence set forth as GenBank accession number NP_663531 (SEQ ID NO: 1), or a variant thereof that differs from same due to a conservative amino acid substitution. In some embodiments, a wildtype mouse TARDBP gene comprises a nucleic acid sequence set forth as GenBank accession number NM_145556.4 (SEQ ID NO:2), or a variant thereof that differs from same due to degeneracy of the genetic code and/or a conservative codon substitution. In some embodiments, a wildtype rat TARDBP gene comprises a nucleotide sequence that encodes a wildtype rat TDP-43 polypeptide comprising an amino acid sequence set forth as GenBank accession number NP_001011979 (SEQ ID NO:3), or a variant thereof that differs from same due to a conservative amino acid substitution. In some embodiments, a wildtype rat TARDBP gene comprises a nucleic acid sequence set forth as GenBank accession number NM_001011979.2 (SEQ ID NO:4), or a variant thereof that differs from same due to degeneracy of the genetic code and/or a conservative codon substitution. In some embodiments, a wildtype human TARDBP gene encodes a TDP-43 polypeptide comprising an amino acid set forth as GenBank accession number NP_031401.1 (SEQ ID NO:5), or a variant thereof that differs from same due to a conservative amino acid substitution. In some embodiments, a wildtype human TARDBP gene comprises a nucleic acid sequence set forth as GenBank accession number NM_007375.3 (SEQ ID NO:6), or a variant thereof that differs from same due to degeneracy of the genetic code and/or a conservative codon substitution.

Described herein is a mutated TARDBP gene. A mutated TARDBP gene may comprise a knockout mutation. A mutated TARDBP gene may encode a mutant TDP-43 polypeptide, wherein the mutant TDP-43 polypeptide lacks a functional domain. For example, a mutated TARDBP gene may comprise a nucleotide sequence encoding a TDP-43 functional domain comprising a point mutation, an insertion within, and/or deletion of a portion or all of the domain, wherein the point mutation, insertion, and/or deletion results in a loss-of-function of the functional domain, and wherein the mutated TARDBP gene still encodes a TDP-43 polypeptide, albeit a mutant TDP-43 polypeptide lacking a functional domain due to the mutation. A polypeptide may be referred to as a mutant TDP-43 polypeptide wherein it comprises at least one wildtype TDP-43 domain or variant thereof and/or wherein it is specifically bound by an anti-TDP-43 antibody or antigen binding portion thereof. Similarly, a mutated TARDBP gene may be so classified wherein the mutated TARDBP gene encodes a mutant TDP-43 polypeptide, e.g., a polypeptide that comprises at least one wildtype TDP-43 domain or variant thereof and/or may be specifically bound by an anti-TDP-43 antibody or antigen binding portion thereof.

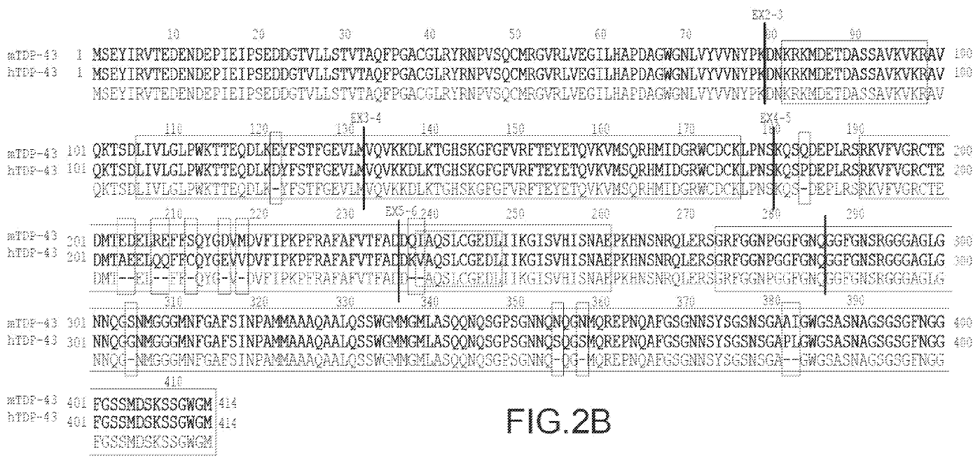

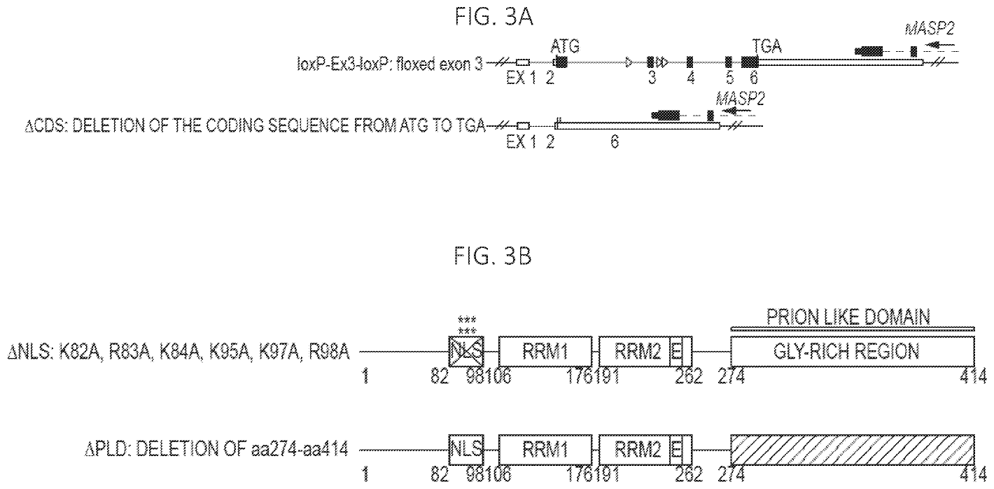

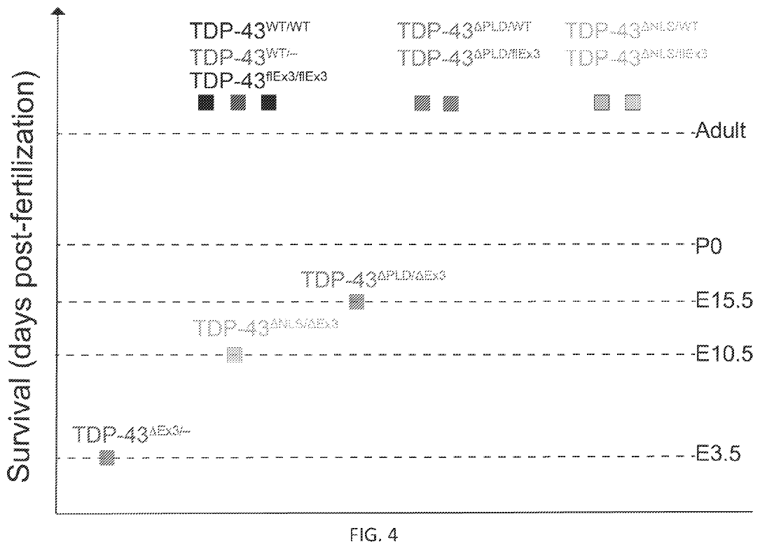

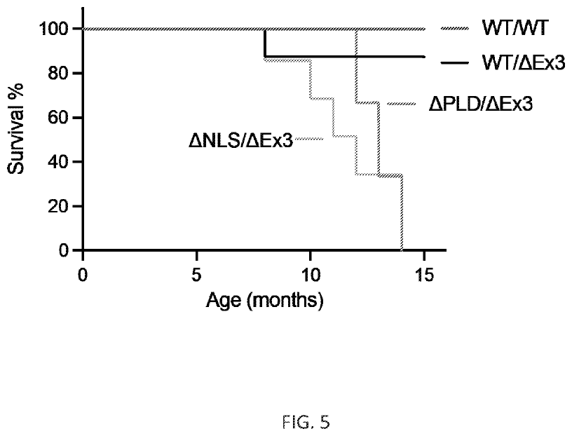

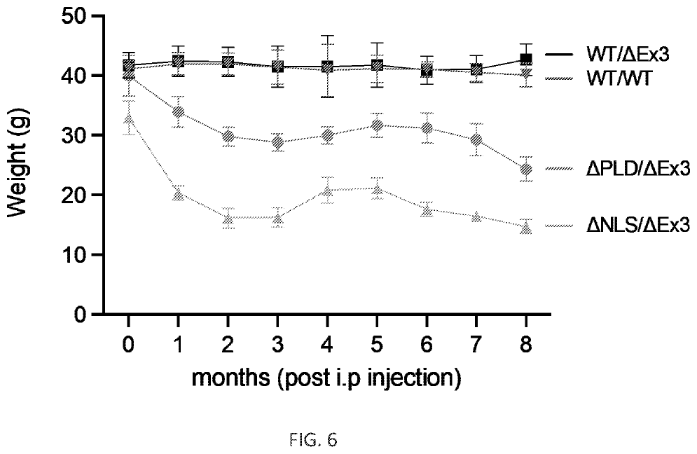

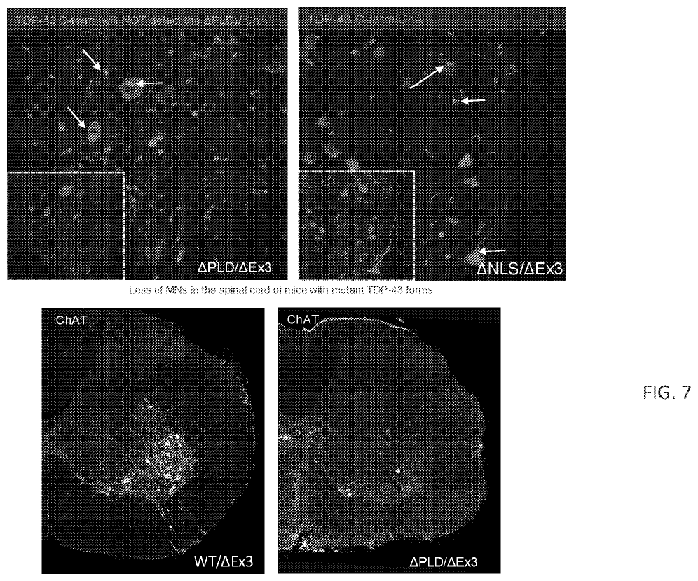

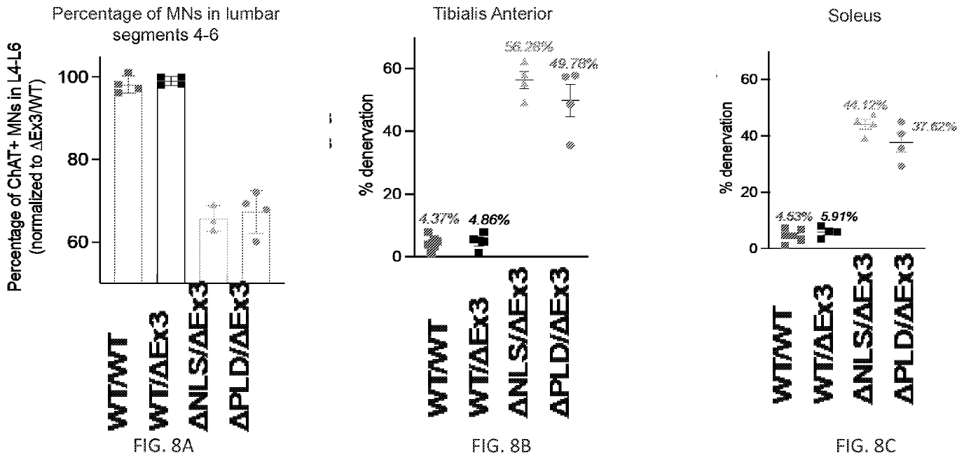

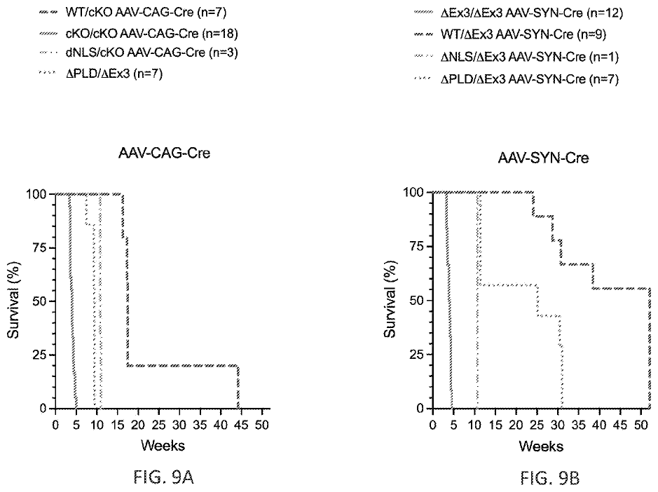

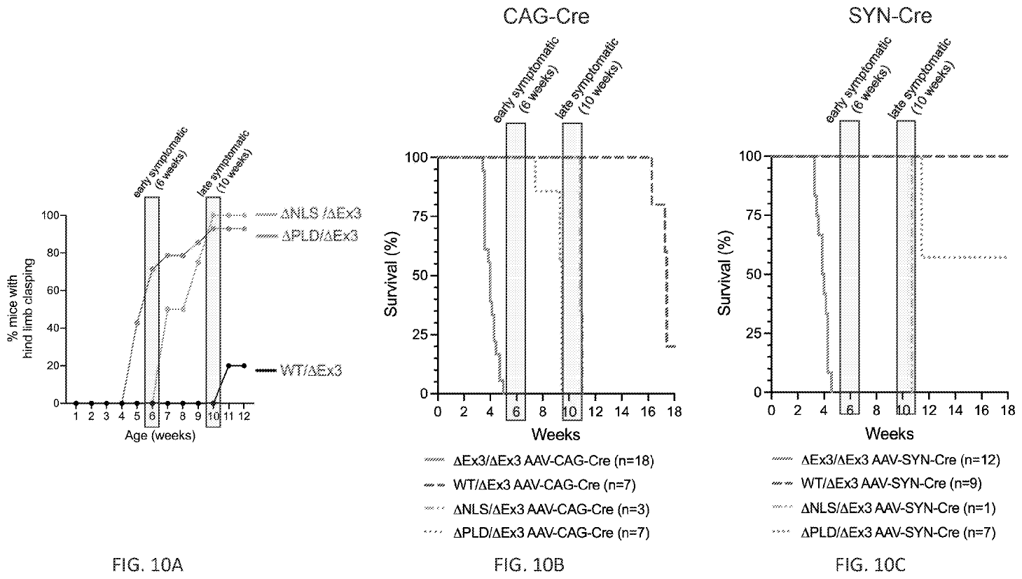

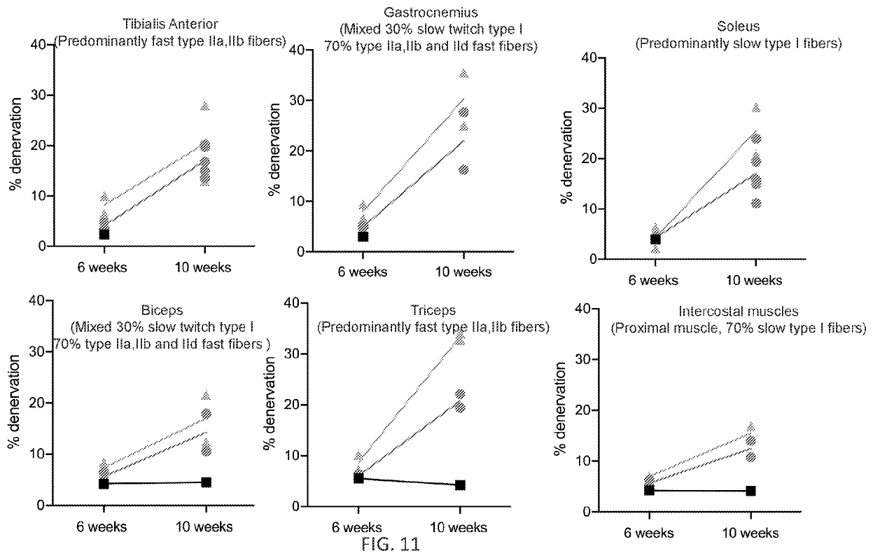

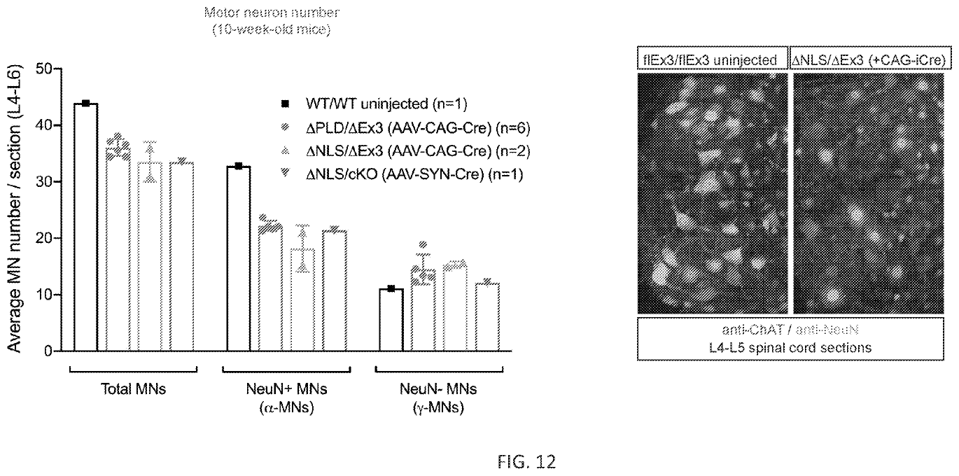

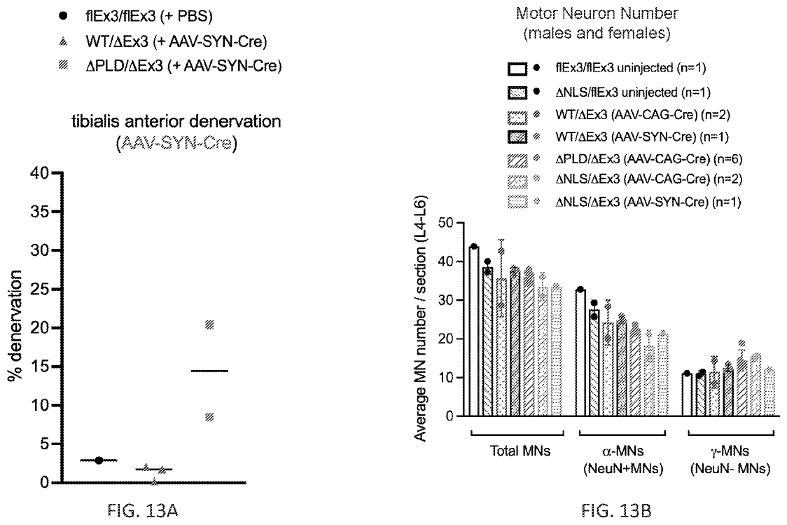

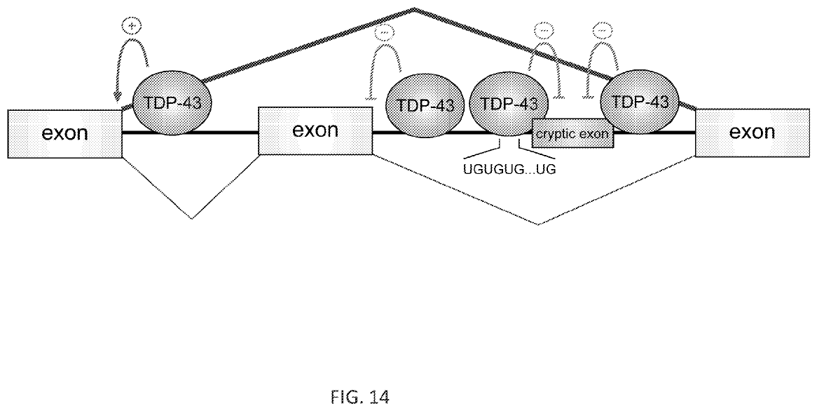

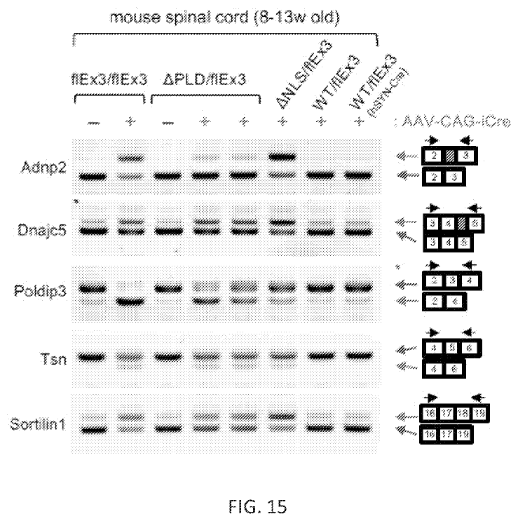

The functional domains of TDP-43 have been identified as a nuclear localization signal (NLS), two RNA recognition motifs (RRM1 and RRM2), a putative nuclear export signal (E), and a glycine rich prion like domain (PLD). See FIGS. 1 and 2. A wildtype TDP-43 polypeptide comprises a TDP-43 NLS at amino acids 82-99, a TDP-43 RRM1 at amino acids 106-176, a TDP-43 RRM2 at amino acids 191-262, a TDP-43 E at amino acids 239-248, and a TDP-43 PLD at amino acids 274-414.

Classical NLS sequences comprise stretches of basic amino acids, primarily lysine (K) and arginine (R) residues, and bipartite NLS comprise two clusters of these basic amino acids separated by a linker region comprising about 10-13 amino acids. An amino acid substitution and/or deletion of a basic amino acid sequence of a classical NLS may abolish function of the classical NLS. McLane and Corbett (2009) IUBMB Life 61:697-706. A TDP-43 NLS comprises lysine and arginine residues at positions 82, 83, 84, 95, 97, and 98. A wildtype TDP-43 polypeptide modified to comprise an amino acid substitution and/or deletion at positions 82, 83, 84, 95, 97, and/or 98 may lack a functional NLS. A mutant TDP-43 polypeptide lacking a functional NLS may comprise an amino acid sequence set forth in SEQ ID NO:1 modified to comprise an amino acid substitution and/or deletion at positions 82, 83, 84, 95, 97, and/or 98. A mutant TDP-43 polypeptide lacking a functional NLS may comprise an amino acid sequence set forth in SEQ ID NO:3 modified to comprise an amino acid substitution and/or deletion at positions 82, 83, 84, 95, 97, and/or 98. A mutant TDP-43 polypeptide lacking a functional NLS may comprise an amino acid sequence set forth in SEQ ID NO:5 modified to comprise an amino acid substitution and/or deletion at positions 82, 83, 84, 95, 97, and/or 98. Accordingly, a mutated TARDBP gene that encodes a mutant TDP-43 protein lacking a functional TDP-43 NLS may comprise a sequence encoding a TDP-43 polypeptide comprising a sequence set forth as SEQ ID NO: 1, SEQ ID NO:3, or SEQ ID NO:5 modified to comprise (i) an amino acid substitution at a position selected from the group consisting of 82, 83, 84, 95, 97, and/or 98, and a combination thereof, and/or (ii) a deletion of any amino acids at and between portions 82 and 98. A mutated TARDBP gene that encodes a mutant TDP-43 protein lacking a functional TDP-43 NLS may comprise a nucleotide sequence encoding an amino acid sequence set forth as SEQ ID NO:1. SEQ ID NO:3 or SEQ ID NO:5 modified to comprise an amino acid substitution selected from the group consisting of K82A K83A, R84A, K95A, K97A, K98A or a combination thereof. A mutated TARDBP gene that encodes a mutant TDP-43 protein lacking a functional TDP-43 NLS may comprise a nucleotide sequence encoding an amino acid sequence set forth as SEQ ID NO:1. SEQ ID NO:3 or SEQ ID NO:5 modified to comprise following amino acid substitutions: K82A K83A, R84A, K95A, K97A, and K98A.

RNA binding by a typical RRM is usually achieved by contacts made between the surface of a four-stranded antiparallel p sheet of the typical RRM and a single stranded RNA. Melamed et al. (2013) RNA 19:1537-1551. Two highly conserved motifs, RNP1 (consensus K/R-G-F/Y-G/A-F/Y-V/I/L-X-F/Y, where X is any amino acid) and RNP2 (consensus I/V/L-F/Y-I/V/L-X-N-L, where X is any amino acid) in the central two 3 strands, are the primary mediators of RNA binding. Melamed et al. (2013), supra.

A TDP-43 RRM1, located at amino acid positions 106-176 of a wildtype TDP-43 polypeptide comprises an RNP2 consensus sequence (LIVLGL; SEQ ID NO:7) located at amino acid positions 106-111 and an RNP1 consensus sequence (KGFGFVRF; SEQ ID NO:8) located at amino acid positions 145-152. Previously, W113, T115, F147, F149, D169, R171, and N179 were identified as critical residues for nucleic acid binding. A wildtype TDP-43 polypeptide modified to comprise (i) an amino acid substitution at a position selected from the group consisting of 113, 115, 147, 149, 169, 171, 179 and any combination thereof, (ii) a deletion or substitution of any amino acids at and between positions 106-176, (iii) a deletion or substitution of any amino acids at and between positions 106-111, (iv) a deletion or substitution of any amino acids at and between of 145-152, or (v) any combination of (i)-(iv), may lack a functional RRM1. A mutant TDP-43 polypeptide lacking a functional RRM1 may comprise a sequence set forth as SEQ ID NO:1 modified to comprise (i) an amino acid substitution at a position selected from the group consisting of 113, 115, 147, 149, 169, 171, 179 and any combination thereof, (ii) a deletion or substitution of any amino acids at and between positions 106-176, (iii) a deletion or substitution of any amino acids at and between positions 106-111, (iv) a deletion or substitution of any amino acids at and between of 145-152, or (v) any combination of (i)-(iv). A mutant TDP-43 polypeptide lacking a functional RRM1 may comprise a sequence set forth as SEQ ID NO:3 modified to comprise (i) an amino acid substitution at a position selected from the group consisting of 113, 115, 147, 149, 169, 171, 179 and any combination thereof, (ii) a deletion or substitution of any amino acids at and between positions 106-176, (iii) a deletion or substitution of any amino acids at and between positions 106-111, (iv) a deletion or substitution of any amino acids at and between of 145-152, or (v) any combination of (i)-(iv). A mutant TDP-43 polypeptide lacking a functional RRM1 may comprise a sequence set forth as SEQ ID NO:5 modified to comprise (i) an amino acid substitution at a position selected from the group consisting of 113, 115, 147, 149, 169, 171, 179 and any combination thereof, (ii) a deletion or substitution of any amino acids at and between positions 106-176, (iii) a deletion or substitution of any amino acids at and between positions 106-111, (iv) a deletion or substitution of any amino acids at and between of 145-152, or (v) any combination of (i)-(iv), Accordingly, a mutated TARDBP gene encoding a mutant TDP-43 polypeptide lacking a functional RRM1 may comprise a nucleotide sequence that encodes a TDP-43 polypeptide comprising an amino acid sequence set forth as SEQ ID NO:1, SEQ ID NO:3, or SEQ ID NO:5 modified to comprise (i) an amino acid substitution at a position selected from the group consisting of 113, 115, 147, 149, 169, 171, 179 and any combination thereof, (ii) a deletion or substitution of any amino acids at and between positions 106-176, (iii) a deletion or substitution of any amino acids at and between positions 106-111, (iv) a deletion or substitution of any amino acids at and between of 145-152 of a wildtype TDP-43 polypeptide, or (v) any combination of (i)-(iv). A mutated TARDBP gene encoding a mutant TDP-43 polypeptide lacking a functional RRM1 may comprise a nucleotide sequence that encodes a TDP-43 polypeptide comprising an amino acid sequence set forth as SEQ ID NO: 1, SEQ ID NO:3, or SEQ ID NO:5 modified to comprise a F147L and/or F149L mutation. A mutated TARDBP gene encoding a mutant TDP-43 polypeptide lacking a functional RRM1 may comprise a nucleotide sequence that encodes a TDP-43 polypeptide comprising an amino acid sequence set forth as SEQ ID NO:1, SEQ ID NO:3, or SEQ ID NO:5 modified as to comprise the following amino acid substitutions: F147L and F149L.

A TDP-43 RRM2, located at amino acid positions 191-262 of a wildtype TDP-43 polypeptide comprises an RNP2 consensus sequence (VFVGRC; SEQ ID NO:9) located at amino acid positions 193-198 and an RNP1 consensus sequence (RAFAFVT; SEQ ID NO: 10) located at amino acid positions 227-233. F194 and F229 may be considered critical residues for nucleic acid binding. A wildtype TDP-43 polypeptide modified to comprise (i) an amino acid substitution at a position selected from the group consisting of 194 and/or229, (ii) a deletion or substitution of any amino acids at and between positions 193-198, (iii) a deletion or substitution of any amino acids at and between positions 227-233, (iv) a deletion or substitution of any amino acids at and between of 191-262, or (v) any combination of (i)-(v), may lack a functional RRM2. A mutant TDP-43 polypeptide lacking a functional RRM2 may comprise a sequence set forth as SEQ ID NO:1 modified to comprise (i) an amino acid substitution at a position selected from the group consisting of 194 and/or 229, (ii) a deletion or substitution of any amino acids at and between positions 193-198, (iii) a deletion or substitution of any amino acids at and between positions 227-233, (iv) a deletion or substitution of any amino acids at and between of 191-262, or (v) any combination of (i)-(iv). A mutant TDP-43 polypeptide lacking a functional RRM2 may comprise a sequence set forth as SEQ ID NO:3 modified to comprise (i) an amino acid substitution at a position selected from the group consisting of 194 and/or 229, (ii) a deletion or substitution of any amino acids at and between positions 193-198, (iii) a deletion or substitution of any amino acids at and between positions 227-233, (iv) a deletion or substitution of any amino acids at and between of 191-262, or (v) any combination of (i)-(iv). A mutant TDP-43 polypeptide lacking a functional RRM2 may comprise a sequence set forth as SEQ ID NO:5 modified to comprise (i) an amino acid substitution at a position selected from the group consisting of 194 and/or 229, (ii) a deletion or substitution of any amino acids at and between positions 193-198, (iii) a deletion or substitution of any amino acids at and between positions 227-233, (iv) a deletion or substitution of any amino acids at and between of 191-262, or (v) any combination of (i)-(iv). Accordingly, a mutated TARDBP gene encoding a mutant TDP-43 polypeptide lacking a functional RRM2 may comprise a nucleotide sequence encoding a TDP-43 polypeptide comprising an amino acid sequence set forth as SEQ ID NO:1, SEQ ID NO:3, or SEQ ID NO:5 modified to comprise (i) an amino acid substitution at positions 194 and/or 229 of a wildtype TDP-43 polypeptide (ii) a deletion or substitution of any amino acids at and between positions 191-262, or (iii) both (i) and (ii). A mutated TARDBP gene encoding a mutant TDP-43 polypeptide lacking a functional RRM2 may comprise a nucleotide sequence encoding a TDP-43 polypeptide comprising an amino acid sequence set forth as SEQ ID NO:1, SEQ ID NO:3, or SEQ ID NO:5 modified to comprise a F194L and/or F229L mutation. A mutated TARDBP gene encoding a mutant TDP-43 polypeptide lacking a functional RRM2 may comprise a nucleotide sequence encoding a TDP-43 polypeptide comprising an amino acid sequence set forth as SEQ ID NO:1, SEQ ID NO:3, or SEQ ID NO:5 modified to comprise a F194L and a F229L mutation.

A nuclear export signal of a wildtype TDP-43 polypeptide may be located at amino acids 239-248. A mutant TDP-43 polypeptide lacking a functional nuclear export signal may comprise an amino acid sequence set forth as SEQ ID NO:1 modified to comprise a deletion of any amino acids at and between positions 236-251. A mutant TDP-43 polypeptide lacking a nuclear export signal may comprise an amino acid sequence set forth as SEQ ID NO:1 modified to comprise a deletion of at least amino acids 239-250. A mutant TDP-43 polypeptide lacking a nuclear export signal may comprise an amino acid sequence set forth as SEQ ID NO:3 modified to comprise a deletion of any amino acids at and between positions 236-251. A mutant TDP-43 polypeptide lacking a nuclear export signal may comprise an amino acid sequence set forth as SEQ ID NO:3 modified to comprise a deletion of at least amino acids 239-250. A mutant TDP-43 polypeptide lacking a nuclear export signal may comprise an amino acid sequence set forth as SEQ ID NO:5 modified to comprise a deletion of any amino acids at and between positions 236-251. A mutant TDP-43 polypeptide lacking a nuclear export signal may comprise an amino acid sequence set forth as SEQ ID NO:5 modified to comprise a deletion of at least amino acids 239-250. Accordingly, a mutated TARDBP gene encoding a mutant TDP-43 polypeptide lacking a functional nuclear export signal may comprise a nucleotide sequence encoding a TDP-43 polypeptide comprising an amino acid sequence set forth as SEQ ID NO:1, SEQ ID NO:3, or SEQ ID NO:5 modified to comprise a deletion of amino acids at and between 236-251, e.g., a deletion of amino acids at and between 239-250.

A prion like domain (PLD) of a wildtype TDP-43 polypeptide may be located at amino acids 274-414. A mutant TDP-43 polypeptide lacking a functional PLD may comprise an amino acid sequence set forth as SEQ ID NO:1 modified to comprise a deletion of at least one or all amino acids at and between positions 274-414. A mutant TDP-43 polypeptide lacking a functional PLD may comprise an amino acid sequence set forth as SEQ ID NO:3 modified to comprise a deletion of at least one or all amino acids at and between positions 274-414. A mutant TDP-43 polypeptide lacking a functional PLD may comprise an amino acid sequence set forth as SEQ ID NO:5 modified to comprise a deletion of at least one or all amino acids at and between positions 274-414. Accordingly, a mutated TARDBP gene that encodes a mutant TDP-43 polypeptide may comprise a nucleotide sequence encoding a TDP-43 polypeptide comprising an amino acid sequence set forth as SEQ ID NO:1, SEQ ID NO:3 or SEQ ID NO:5 modified to comprise a deletion of at least one or all amino acids at and between positions 274-414.

A mutated TARDBP gene may comprise a structure illustrated in FIG. 3A. A mutated TARDBP gene may encode a mutant TDP-43 polypeptide depicted in FIG. 3A.

### Methods of Making Cells and Non-Human Animals Comprising and Expressing a Mutant TARDBP Gene

As outlined above, methods and compositions are provided herein to allow for the targeted genetic modification of a TARDBP locus, e.g., for making an animal or a cell comprising a mutated TARDBP gene and/or for evaluating the biological function of a TDP-43 domain. It is further recognized that additional targeted genetic modification can be made. Such systems that allow for these targeted genetic modifications can employ a variety of components and for ease of reference, herein the term “targeted genomic integration system” generically includes all the components required for an integration event (i.e., the various nuclease agents, recognition sites, insert DNA polynucleotides, targeting vectors, target genomic locus, etc.).

A method of making a non-human animal or non-human animal cell that expresses a mutant TDP-43 polypeptide and/or for evaluating the biological function of a TDP-43 domain may comprise modifying the genome of the non-human animal cell to comprise a mutated TARDBP gene. The mutated TARDBP gene may encode the mutant TDP-43 polypeptide, wherein the mutant TDP-43 polypeptide lacks the functional domain.

A method of making a non-human animal or a non-human animal cell that expresses a mutant TDP-43 polypeptide and/or for evaluating the biological function of a TDP-43 domain may comprise modifying the genome of the non-human animal or cell to comprise a mutated TARDBP gene, wherein the mutated TARDBP gene comprises a knockout mutation. In some embodiments, the non-human animal cell is a non-human animal embryonic stem cell.

The methods provided herein may comprise introducing into a cell one or more polynucleotides or polypeptide constructs comprising the various components of the targeted genomic integration system. “Introducing” means presenting to the cell the sequence (polypeptide or polynucleotide) in such a manner that the sequence gains access to the interior of the cell. The methods provided herein do not depend on a particular method for introducing any component of the targeted genomic integration system into the cell, only that the polynucleotide gains access to the interior of a least one cell. Methods for introducing polynucleotides into various cell types are known in the art and include, but are not limited to, stable transfection methods, transient transfection methods, and virus-mediated methods.

In some embodiments, the cells employed in the methods and compositions have a DNA construct stably incorporated into their genome. “Stably incorporated” or “stably introduced” means the introduction of a polynucleotide into the cell such that the nucleotide sequence integrates into the genome of the cell and is capable of being inherited by progeny thereof. Any protocol may be used for the stable incorporation of the DNA constructs or the various components of the targeted genomic integration system.

Transfection protocols as well as protocols for introducing polypeptides or polynucleotide sequences into cells may vary. Non-limiting transfection methods include chemical-based transfection methods include the use of liposomes; nanoparticles; calcium phosphate (Graham el al. (1973). Virology 52 (2): 456-67, Bacchetti ei al. (1977) Proc Natl Acad Sci USA 74 (4): 1590-4 and, Kriegler, M (1991). Transfer and Expression: A Laboratory Manual. New York: W. H. Freeman and Company. pp. 96-97); dendrimers; or cationic polymers such as DEAE-dextran or polyethylenimine. Non chemical methods include electroporation; Sono-poration; and optical transfection. Particle-based transfections include the use of a gene gun, magnet assisted transfection (Bertram, J. (2006) Current Pharmaceutical Biotechnology 7, 277-28). Viral methods can also be used for transfection.

Cells comprising a mutated TARDBP gene can be generated by employing the various methods disclosed herein. Modifying may comprise replacing an endogenous TARDBP gene with the mutated TARDBP gene that encodes the mutant TDP-43 polypeptide and/or replacing an endogenous TARDBP gene with a TARDBP gene comprising a knockout mutation, such as a conditional knockout mutation. Modifying may comprise culturing the cell in conditions that eliminates expression of the TARDBP gene comprising a knockout mutation. Conditions that may eliminate the expression of a TARDBP gene may include expressing a recombinase protein, e.g., Cre-recombinase.

Such modifying methods may comprise (1) integrating a mutated TARDBP gene at the target TARDBP genomic locus of interest of a pluripotent cell of a non-human animal to generate a genetically modified pluripotent cell comprising the mutated TARDBP gene in the targeted TARDBP genomic locus employing the methods disclosed herein; and (2) selecting the genetically modified pluripotent cell having the mutated TARDBP gene at the target TARDBP genomic locus. Animals may be further generated by (3) introducing the genetically modified pluripotent cell into a host embryo of the non-human animal, e.g., at a pre-morula stage; and (4) implanting the host embryo comprising the genetically modified pluripotent cell into a surrogate mother to generate an F0 generation derived from the genetically modified pluripotent cell. The non-human animal can be a non-human mammal, a rodent, a mouse, a rat, a hamster, a monkey, an agricultural mammal or a domestic mammal, or a fish or a bird.

The pluripotent cell can be a human ES cell, a non-human ES cell, a rodent ES cell, a mouse ES cell, a rat ES cell, a hamster ES cell, a monkey ES cell, an agricultural mammal ES cell or a domesticated mammal ES cell. In other embodiments, the pluripotent cell is a non-human cell, a mammalian cell, a human cell, a non-human mammalian cell, a human pluripotent cell, a human ES cell, a human adult stem cell, a developmentally-restricted human progenitor cell, a human iPS cell, a rodent cell, a rat cell, a mouse cell, a hamster cell. In one embodiment, the targeted genetic modification results in a mutated TARDBP gene.

A mouse pluripotent cell, totipotent cell, or host embryo can be from any strain of mouse including, for example, inbred strains, hybrid strains, and outbred strains. Examples of mouse strains include a 129 strain, a C57BL strain (e.g., a C57BL/6 strain), a mix of 129 and C57BL/6 (e.g., 50% 129 and 50% C57BL/6), a BALB/c strain, and a Swiss Webster strain. Examples of 129 strains include 129P1, 129P2, 129P3, 129X1, 129S1 (e.g., 12951/SV, 12951/SvIm), 12952, 12954, 12955, 12959/SvEvH, 12956 (129/SvEvTac), 12957, 12958, 129T1, and 129T2 (see, e.g., Festing et al. (1999) Revised nomenclature for strain 129 mice, Mammalian Genome 10:836). Examples of C57BL strains include C57BL/A, C57BL/An, C57BL/GrFa, C57BL/KaLwN, C57BL/6, C57BL/6J, C57BL/6ByJ, C57BL/6NJ, C57BL/10, C57BL/lOScSn, C57BL/10Cr, and C57BL/Ola. Mice can be mixes of an aforementioned 129 strain (e.g., a 129S6 (129/SvEvTac) strain) and an aforementioned C57BL/6 strain, mixes of one or more aforementioned 129 strains, or mixes of one or more aforementioned C57BL strains. Mice can also be from a strain excluding 129 strains.

A rat pluripotent cell, totipotent cell, or host embryo can be from any rat strain, including, for example, inbred strains, hybrid strains, and outbred strains. Examples of rat strains include an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rat pluripotent cells, totipotent cells, or host embryos can also be obtained from a strain derived from a mix of two or more strains recited above. For example, the rat pluripotent cell, totipotent cell, or host embryo can be derived from a strain selected from a DA strain and an ACI strain. The ACI rat strain is characterized as having black agouti, with white belly and feet and an RT1aI haplotype. Such strains are available from a variety of sources including Harlan Laboratories. An example of a rat ES cell line from an ACI rat is the ACJ.G1 rat ES cell. The Dark Agouti (DA) rat strain is characterized as having an agouti coat and an RT1^{av}1 haplotype. Such rats are available from a variety of sources including Charles River and Harlan Laboratories. Examples of a rat ES cell line from a DA rat and are the DA.2B rat ES cell line or the DA.2C rat ES cell line. Other examples of rat strains are provided, for example, in US 2014/0235933, US 2014/0310828, and US 2014/0309487, each of which is herein incorporated by reference in its entirety for all purposes.

For example, germline-transmittable rat ES cells can be obtained by culturing isolated rat ES cells on a feeder cell layer with a medium comprising N2 supplement, B27 supplement, about 50 U/mL to about 150 U/mL leukemia inhibitory factor (LIF), and a combination of inhibitors consisting of a MEK inhibitor and a GSK3 inhibitor, wherein the feeder cell layer is not modified to express LIF, and wherein the rat ES cells: (i) have been modified to comprise a targeted genetic modification comprising at least one insertion of a heterologous polynucleotide comprising a selection marker into the genome of the rat ES cells and are capable of transmitting the targeted genetic modification through the germline; (ii) have a normal karyotype; (iii) lack expression of c-Myc; and (iv) form spherical, free-floating colonies in culture (See, for example, US 2014-0235933 A1 and US 2014-0310828 A1, each of which is incorporated by reference in its entirety). Other examples of derivation of rat embryonic stem cells and targeted modification are provided, e.g., in Yamamoto et al. (“Derivation of rat embryonic stem cells and generation of protease-activated receptor-2 knockout rats,” Transgenic Res. 21:743-755, 2012) and Kwamata and Ochiya (“Generation of genetically modified rats from embryonic stem cells,” Proc. Natl. Acad. Sci. USA 107(32):14223-14228, 2010).

Nuclear transfer techniques can also be used to generate the non-human animals. Briefly, methods for nuclear transfer include the steps of: (1) enucleating an oocyte; (2) isolating a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods oocytes are generally retrieved from deceased animals, although they may be isolated also from either oviducts and/or ovaries of live animals. Oocytes can be matured in a variety of medium known to those of ordinary skill in the art prior to enucleation. Enucleation of the oocyte can be performed in a number of manners well known to those of ordinary skill in the art. Insertion of the donor cell or nucleus into the enucleated oocyte to form a reconstituted cell is usually by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell is typically activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, are typically cultured in medium well known to those of ordinary skill in the art and then transferred to the womb of an animal. See, for example, US20080092249, WO/1999/005266A2, US20040177390, WO/2008/017234A1, and U.S. Pat. No. 7,612,250, each of which is herein incorporated by reference.

Other methods for making a non-human animal comprising in its germline one or more genetic modifications as described herein is provided, comprising: (a) modifying a targeted genomic TARDBP locus of a non-human animal in a prokaryotic cell employing the various methods described herein; (b) selecting a modified prokaryotic cell comprising the genetic modification at the targeted genomic locus; (c) isolating the genetically modified targeting vector from the genome of the modified prokaryotic cell; (d) introducing the genetically modified targeting vector into a pluripotent cell of the non-human animal to generate a genetically modified pluripotent cell comprising the insert nucleic acid at the targeted TARDBP genomic locus; (e) selecting the genetically modified pluripotent cell; (f) introducing the genetically modified pluripotent cell into a host embryo of the non-human animal at a pre-morula stage; and (g) implanting the host embryo comprising the genetically modified pluripotent cell into a surrogate mother to generate an F0 generation derived from the genetically modified pluripotent cell. In such methods the targeting vector can comprise a large targeting vector. The non-human animal can be a non-human mammal, a rodent, a mouse, a rat, a hamster, a monkey, an agricultural mammal or a domestic mammal. The pluripotent cell can be a human ES cell, a non-human ES cell, a rodent ES cell, a mouse ES cell, a rat ES cell, a hamster ES cell, a monkey ES cell, an agricultural mammal ES cell or a domestic mammal ES cell. In other embodiments, the pluripotent cell is a non-human cell, a mammalian cell, a human cell, a non-human mammalian cell, a human pluripotent cell, a human ES cell, a human adult stem cell, a developmentally-restricted human progenitor cell, a human iPS cell, a human cell, a rodent cell, a rat cell, a mouse cell, a hamster cell. In one embodiment, the targeted genetic modification results in a mutated TARDBP gene, e.g., a mutant TARDBP gene that encodes a mutant TDP-43 polypeptide lacking a functional domain and/or a mutant TARDBP gene comprising a knockout mutation.

In further methods, the isolating step (c) further comprises (ci) linearizing the genetically modified targeting vector (i.e., the genetically modified LTVEC). In still further embodiments, the introducing step (d) further comprises (dl) introducing a nuclease agent into the pluripotent cell to facilitate homologous recombination. In one embodiment, selecting steps (b) and/or (e) are carried out by applying a selectable agent as described herein to the prokaryotic cell or the pluripotent cell. In one embodiment, selecting steps (b) and/or (e) are carried out via a modification of allele (MOA) assay as described herein.

In some embodiments, various genetic modifications of the target genomic loci described herein can be carried out by a series of homologous recombination reactions (BHR) in bacterial cells using an LTVEC derived from Bacterial Artificial Chromosome (BAC) DNA using VELOCIGENE® genetic engineering technology (see, e.g., U.S. Pat. No. 6,586,251 and Valenzuela, D. M. et al. (2003), Nature Biotechnology 21(6): 652-659, which is incorporated herein by reference in their entireties).

In some embodiments, the targeted pluripotent and/or totipotent cells comprising various genetic modifications as described herein are used as insert donor cells and introduced into a pre-morula stage embryo from a corresponding organism, e.g., an 8-cell stage mouse embryo, via the VELOCIMOUSE@method (see, e.g., U.S. Pat. Nos. 7,576,259, 7,659,442, 7,294,754, and US 2008-0078000 A1, all of which are incorporated by reference herein in their entireties). The non-human animal embryo comprising the genetically modified pluripotent and/or totipotent cells is incubated until the blastocyst stage and then implanted into a surrogate mother to produce an F0 generation. In some embodiments, targeted mammalian ES cells comprising various genetic modifications as described herein are introduced into a blastocyst stage embryo. Non-human animals bearing the genetically modified genomic locus (i.e. a TARDBP locus) can be identified via modification of allele (MOA) assay as described herein. The resulting F0 generation non-human animal derived from the genetically modified pluripotent and/or totipotent cells is crossed to a wild-type non-human animal to obtain F1 generation offspring. Following genotyping with specific primers and/or probes, F1 non-human animals that are heterozygous for the genetically modified genomic locus may be crossed to each other to produce F2 generation non-human animal offspring that are homozygous for the genetically modified genomic locus.

In some embodiments, a method for making an animal further comprises administering to an animal (e.g., progeny heterozygous for a mutant TARDBP gene on one chromosome and for a conditional knockout TARDBP gene on the other homologous chromosome as described herein) a site-specific recombinase to create a knockout or null-mutation at an endogenous TARDBP locus comprising a TARDBP gene that comprises a conditional knockout mutation, e.g., wherein one or more exons of the TARDBP gene is flanked by a site-specific recombinase recognition sequence. In some embodiments, the site-specific recombinase is administered neonatally, e.g., at P0-P10 after birth. In some embodiments, the site-specific recombinase is administered at or after about 2 weeks after birth. In some embodiments, the site-specific recombinase is administered at or after about 3 weeks after birth. In some embodiments, the site-specific recombinase is administered at or after about 4 weeks after birth. In some embodiments, the site-specific recombinase is administered at or after about 5 weeks after birth. In some embodiments, the site-specific recombinase is administered at or after about 6 weeks after birth. In some embodiments, the site-specific recombinase is administered at or after about 7 weeks after birth. In some embodiments, the site-specific recombinase is administered at or after about 2 months after birth. In some embodiments, the site-specific recombinase is administered at or after about 3 months after birth. In some embodiments, the site-specific recombinase is administered at or after about 4 months after birth. In some embodiments, the site-specific recombinase is administered at or after about 5 months after birth. In some embodiments, the site-specific recombinase is administered at or after about 6 months after birth.

Methods of administering a site-specific recombinase to an animal are well-known in the art. In some embodiments, administering a site-specific recombinase comprises injecting a viral vector, e.g., an adeno-associated viral vector, into the animal, wherein the viral vector comprises a viral genome that encodes a recombinase gene. In some embodiments, the viral genome comprises the recombinase gene operably linked to a promoter. In some embodiments, the promoter is a tissue specific promoter. In some embodiments, the promoter is a neuron specific promoter. In some embodiments, the promoter is a synapsin promoter.

Methods of making such heterozygous mice are well-known in the art. In some embodiments, a cell comprising a mutated TARDBP gene may be made by modifying an ES cell to comprise the mutated TARDB gene and culturing in vitro the ES cell in differentiating medium. In some embodiments, culturing in vitro the ES cell comprises differentiating the ES cell into primitive ectoderm cells or embryonic stem cell derived motor neurons (ESMNs).

In one embodiment, a method for making a cell or animal comprising a mutated TARDBP gene is provided. Such methods comprise: (a) contacting a pluripotent cell with a targeting construct comprising a mutated TARDBP gene or a mutated portion thereof flanked by 5′ and 3′ homology arms; wherein the targeting construct undergoes homologous recombination with the TARDBP locus in a genome of the cell to form a modified pluripotent cell. Methods of making a non-human animal further comprises (b) introducing the modified pluripotent cell into a host embryo; and (c) gestating the host embryo in a surrogate mother, wherein the surrogate mother produces progeny comprising a modified TARDBP locus, wherein said genetic modification results in a mutant TDP-43 polypeptide lacking a functional domain.

### Cells and Animals

The cells (which may be comprised within non-human animal tissues or non-human animals) disclosed herein may be any type of cell comprising a mutated TARDBP gene as disclosed herein. A cell may comprise a mutated non-human animal TARDBP gene (e.g., a mutated TARDBP gene of the non-human animal) or a mutated human TARDBP gene.

A non-human animal or cell may comprise a mutated TARDBP gene that encodes a mutant TDP-43 polypeptide, wherein the mutant TDP-43 polypeptide lacks a functional domain, and wherein the cell expresses the mutant TDP-43 polypeptide. For example, a cell may comprise a mutated TARDBP gene that encodes a mutant TDP-43 polypeptide lacking a functional domain comprising the nuclear localization signal (NLS), the prion like domain (PLD), or a combination thereof. A cell may comprise a mutated TARDBP gene that encodes a mutant TDP-43 polypeptide lacking a functional domain due to one or more of the following: (a) a point mutation of an amino acid in the NLS (e.g., K82A K83A, R84A, K95A, K97A, K98A or a combination thereof) and/or (b) a deletion of at least a portion of the prion-like domain (e.g., a deletion of the amino acids at and between positions 274 and 414 of a wildtype TDP-43 polypeptide). A non-human animal or cell may comprise a mutated TARDBP gene that encodes a mutant TDP-43 polypeptide comprising the following mutations: K82A K83A, R84A, K95A, K97A, and K98A, wherein the mutant TDP-43 polypeptide lacks a functional NLS. A non-human animal or cell may comprise a mutated TARDBP gene that encodes a mutant TDP-43 polypeptide comprising a deletion between and including the amino acids at positions 274 to 414 of a wildtype TDP-43 polypeptide, wherein the mutant TDP-43 polypeptide lacs a functional PLD.

A cell or animal may comprise a mutated TARDBP gene comprising a knockout mutation, e.g., a conditional knockout mutation, a deletion of the entire coding sequence of the TARDBP gene, etc. A cell or animal may comprise a mutated TARDBP gene comprising a conditional knockout mutation, e.g., the mutated TARDBP gene may comprise site-specific recombination recognition sequence.

Site-specific recombinases include enzymes that can facilitate recombination between recombinase recognition sites, where the two recombination sites are physically separated within a single nucleic acid or on separate nucleic acids. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

A cell or animal may comprise a mutated TARDBP gene comprising a loxP sequence flanking an exon comprising a TDP-43 coding sequence, e.g., exon 3. A cell may comprise a mutated TARDBP gene comprising a loxP sequence and lacking a TDP-43 coding sequence, e.g., exon 3. A cell may comprise a mutated TARDBP gene lacking the entire TDP-43 coding sequence, e.g., a mutated TARDBP gene comprising a deletion of the entire coding sequence of a TDP-43 polypeptide.

In some embodiments, the cell or animal may comprise the mutated TARDBP gene inserted at the endogenous TARDBP locus, e.g., in its germline genome. In some embodiments, a cell comprises a mutated TARDBP gene, e.g., mutated TARDBP gene comprising a knockout mutation and/or a mutated TARDBP gene that encodes a mutant TDP-43 polypeptide, that replaces an endogenous TARDBP gene at an endogenous TARDBP locus. In some embodiments, a mutated TARDBP gene is operably linked to an endogenous TARDBP promoter and/or regulatory element.

The cell or animal may be heterozygous or homozygous for a mutated TARDBP gene. A diploid organism has two alleles, one at each genetic locus of the pair of homologous chromosomes. Each pair of alleles represents the genotype of a specific genetic locus. Genotypes are described as homozygous if there are two identical alleles at a particular locus and as heterozygous if the two alleles differ.

A cell or animal may comprise (i) at an endogenous TARDBP locus, a replacement of an endogenous TARDBP gene with a mutated TARDBP gene that encodes a mutant TDP-43 polypeptide, and (ii) at the other endogenous TARDPP locus of a homologous chromosome, a mutated TARDBP gene comprising a conditional knockout mutation and/or a knockout mutation.

A cell or animal comprising a mutated TARDBP gene may express the mutant TDP-43 polypeptide encoded therefrom. A cell or animal comprising a mutated TARDBP gene and expressing a mutant TDP-43 polypeptide encoded therefrom may, or may not, express a wildtype TDB-43 polypeptide.

A cell or animal comprising a mutated TARDBP gene may express the mutant TDP-43 polypeptide encoded therefrom and may be characterized by one or more of the following (i) a level of mRNA transcripts of the mutated TARDBP gene that is comparable to the level of mRNA transcript levels of a wildtype TARDBP gene in a control cell, (ii) increased levels of the mutant TDP-43 polypeptide compared to levels of wildtype TDP-43 polypeptide in a control cell, (iii) the mutant TDP-43 polypeptide is found at a higher concentration in the cytoplasm than in the nucleus of the cell, (iv) the mutant TDP-43 polypeptide exhibits increased insolubility compared to a wildtype TDP-43 polypeptide, (v) cytoplasmic aggregates comprising the mutant TDP-43 polypeptide, (vi) increased splicing of cryptic exons of genes compared to that of cells expressing a wildtype TDP-43, (vii) decreased levels of an alternatively spliced TDP-43 mRNA lacking a sequence encoding a TDP-43 PLD.

The cells may be cultured in vitro, may be examined ex vivo, or in vivo. For example, the cells can be in vivo within an animal.

The cells may be eukaryotic cells, which include, for example, fungal cells (e.g., yeast), plant cells, animal cells, mammalian cells, non-human mammalian cells, and human cells. The term “animal” includes any member of the animal kingdom, including, for example, mammals, fishes, reptiles, amphibians, birds, and worms. A mammalian cell can be, for example, a non-human mammalian cell, a rodent cell, a rat cell, a mouse cell, or a hamster cell. Other non-human mammals include, for example, non-human primates, monkeys, apes, orangutans, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans. In some embodiments, an animal can be a human or a non-human animal, including, but not limited to, mice, rats, rabbits, dogs, cats, pigs, and non-human primates, including, but not limited to, monkeys and chimpanzees. In some embodiments, a non-human animal cell is a rodent cell, e.g., a rat cell or a mouse cell.

Non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Svlm), 129S2, 129S4, 12955, 12959/SvEvH, 129S6 (129/SvEvTac), 129S7, 12958, 129T1, and 129T2. See, e.g., Festing et al. (1999) Mammalian Genome 10:836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/1OScSn, C57BL/1OCr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{a}v1 haplotype. Such strains are available from a variety of sources including Harlan Laboratories. The Dark Agouti (DA) rat strain is characterized as having an agouti coat and an RTIa¹ haplotype. Such rats are available from a variety of sources including Charles River and Harlan Laboratories. Some suitable rats can be from an inbred rat strain. See, e.g., US 2014/0235933, herein incorporated by reference in its entirety for all purposes.

The cells can also be any type of undifferentiated or differentiated state. For example, a cell may be a totipotent cell, a pluripotent cell (e.g., a human pluripotent cell or a non-human pluripotent cell such as a mouse embryonic stem (ES) cell or a rat ES cell), or a non-pluripotent cell. Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm).

The cells provided herein can also be germ cells (e.g., sperm or oocytes). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell.

Suitable cells provided herein also include primary cells. Primary cells include cells or cultures of cells that have been isolated directly from an organism, organ, or tissue. Primary cells include cells that are neither transformed nor immortal. They include any cell obtained from an organism, organ, or tissue which was not previously passed in tissue culture or has been previously passed in tissue culture but is incapable of being indefinitely passed in tissue culture.

Other suitable cells provided herein include immortalized cells. Immortalized cells include cells from a multicellular organism that would normally not proliferate indefinitely but, due to mutation or alteration, have evaded normal cellular senescence and instead can keep undergoing division. Such mutations or alterations can occur naturally or be intentionally induced. Numerous types of immortalized cells are well known. Immortalized or primary cells include cells that are typically used for culturing or for expressing recombinant genes or proteins.

The cells provided herein also include one-cell stage embryos (i.e., fertilized oocytes or zygotes). Such one-cell stage embryos can be from any genetic background (e.g., BALB/c, C57BL/6, 129, or a combination thereof for mice), can be fresh or frozen, and can be derived from natural breeding or in vitro fertilization.

A non-human animal herein may be heterozygous for a mutated TARDBP gene as described herein and a TARDBP gene comprising a conditional knockout mutation that comprises one or more exons flanked by a site-specific recombinase recognition sequence, and have been subjected to a recombinase that recognizes the site-specific recombinase recognition sequence, e.g., in a tissue specific manner. Some non-human animals described herein thus comprise a central nervous system (CNS) comprising a plurality of cells that each comprises: (a) a mutated TARDBP gene at one chromosome at an endogenous TARDBP locus, wherein the mutated IARDBP gene comprises a wildtype TARDBP gene sequence that comprises a mutation in a nuclear localization signal (NLS) encoding sequence or a prion like domain (PLD) encoding sequence such that the mutated TARDBP gene encodes a mutant TDP-43 polypeptide that lacks a functional NLS or a functional PLD, and (b) a knockout TARDBP gene at the other homologous chromosome at an endogenous TARDBP locus, wherein the knockout TARDBP gene comprises the wildtype TARDBP gene sequence that comprises a loss-of-function mutation, and optionally a second plurality of cells, wherein each of the second plurality of cells comprises: (a) the mutated TARDBP gene on one chromosome at an endogenous TARDBP locus, and (b) a conditional knockout TARDBP gene at the other homologous chromosome at an endogenous TARDBP locus, wherein the conditional knockout TARDBP gene comprises the wildtype TARDBP gene sequence with at least one exon flanked by a site-specific recombinase recognition sequence and encodes a wildtype TDP-43 protein, and wherein recognition of the site-specific recombinase recognition sequence by a recombinase results in the deletion of at least one exon and formation of the knockout TARDBP gene. The non-human animal may also further comprises a nucleic acid comprising a sequence that encodes a recombinase, wherein the nucleic acid further comprises (i) a promoter sequence that drives the expression of the recombinase, (ii) a reporter gene sequence, optionally wherein the reporter gene sequence is operably linked to the recombinase gene sequence by a poly A sequence, (iii) an adeno-associated virus (AAV) inverted terminal repeat (ITR) sequence at the 5′ and 3′ ends of the nucleic acid, or (iv) any combination of (i)-(iii).

Any reporter (or detectable moiety) can be used in the methods and compositions provided herein. Non-liming examples of reporters include, for example, β-galactosidase (encoded by the lacZ gene), Green Fluorescent Protein (GFP), enhanced Green Fluorescent Protein (eGFP), mPlum, mCherry, tdTomato, mStrawberry, J-Red, DsRed, mOrange, mKO, mCitrine, Venus, YPet, enhanced yellow fluorescent protein (EYFP), Emerald, CyPet, cyan fluorescent protein (CFP), Cerulean, T-Sapphire, luciferase, alkaline phosphatase, or a combination thereof.

In one embodiment, the promoter is an inducible promoter. In one embodiment, the inducible promoter is a chemically-regulated promoter. In one embodiment, the chemically-regulated promoter is an alcohol-regulated promoter. In one embodiment, the alcohol-regulated promoter is an alcohol dehydrogenase (alcA) gene promoter. In one embodiment, the chemically-regulated promoter is a tetracycline-regulated promoter. In one embodiment, the tetracycline-regulated promoter is a tetracycline-responsive promoter. In one embodiment, the tetracycline-regulated promoter is a tetracycline operator sequence (tetO). In one embodiment, the tetracycline-regulated promoter is a tet-On promoter. In one embodiment, the tetracycline-regulated promoter a tet-Off promoter. In one embodiment, the chemically-regulated promoter is a steroid regulated promoter. In one embodiment, the steroid regulated promoter is a promoter of a rat glucocorticoid receptor. In one embodiment, the steroid regulated promoter is a promoter of an estrogen receptor. In one embodiment, the steroid-regulated promoter is a promoter of an ecdysone receptor. In one embodiment, the chemically-regulated promoter is a metal-regulated promoter. In one embodiment, the metal-regulated promoter is a metalloprotein promoter. In one embodiment, the inducible promoter is a physically-regulated promoter. In one embodiment, the physically-regulated promoter is a temperature-regulated promoter. In one embodiment, the temperature-regulated promoter is a heat shock promoter. In one embodiment, the physically-regulated promoter is a light-regulated promoter. In one embodiment, the light-regulated promoter is a light-inducible promoter. In one embodiment, the light-regulated promoter is a light-repressible promoter.

In one embodiment, the promoter is a tissue-specific promoter. In one embodiment, the promoter is a neuron-specific promoter. In one embodiment, the promoter is a glia-specific promoter.

The site-specific recombinase can be introduced into the cell by any means, including by introducing the recombinase polypeptide into the cell or by introducing a polynucleotide encoding the site-specific recombinase into the host cell.

### Methods Employing a System Expressing a Mutant TDP-43 Polypeptide

Cells and non-human animals comprising a mutated TARDBP gene and expressing a mutant TDP-43 polypeptide lacking a functional domain encoded therefrom as described herein (and tissues or animals comprising such cells) provide a model for studying the function of TDP-43 and/or TDP-43 proteinopathies. For example, cells or non-human animals comprising a mutated TARDBP gene and expressing a mutant TDP-43 polypeptide encoded therefrom lacking a functional domain may exhibit phenotypes characteristic of TDP-43 proteinopathy. In some embodiments, cells, e.g., (a) embryonic stem cell derived motor neurons (ESMNs) comprising a mutated TARDBP gene and expressing a mutant TDP-43 polypeptide encoded therefrom lacking a functional domain and/or (b) isolated from non-human animals comprising at an endogenous TARDBP locus a replacement of the endogenous TARDBP gene with a mutated TARDBP gene and expressing a mutant TDP-43 polypeptide therefrom, may be characterized by one or more of the following (i) a level of mRNA transcripts of the mutated TARDBP gene that is comparable to the level of mRNA transcript levels of a wildtype TARDBP gene in a control cell, (ii) increased levels of the mutant TDP-43 polypeptide compared to levels of wildtype TDP-43 polypeptide in a control cell, (iii) the mutant TDP-43 polypeptide is found at a higher concentration in the cytoplasm than in the nucleus of the cell, (iv) the mutant TDP-43 polypeptide exhibits increased insolubility compared to a wildtype TDP-43 polypeptide, (v) cytoplasmic aggregates comprising the mutant TDP 43 polypeptide, (vi) increased splicing of cryptic exons of genes compared to that of cells expressing a wildtype TDP-43, (vii) decreased levels of an alternatively spliced TDP-43 mRNA lacking a sequence encoding a TDP-43 PLD.

Thus, cells comprising a mutated TARDBP gene and expressing a mutant TDP-43 polypeptide lacking a functional domain encoded therefrom as described herein (and tissues or animals comprising such cells) also provide a system for identifying a therapeutic candidate agent for treating, preventing and/or inhibiting one or more symptoms of TDP-43 proteinopathy (e.g., cytoplasmic accumulation of the mutant TDP-43 polypeptide) and/or restoring the biological functions of a wildtype TDP-43 polypeptide (e.g., repression of cryptic exon splicing and/or increasing the levels of the alternative spliced TDP-43 mRNA). In some embodiments, an effect of a therapeutic agent is determined by contacting a cell comprising a mutated TARDBP gene and expressing a mutant TDP-43 polypeptide lacking a functional domain encoded therefrom with the therapeutic candidate agent. Contacting may be performed in vitro. Contacting may comprise administering to an animal the therapeutic candidate agent.

In some embodiments, performing an assay includes determining the effect on the phenotype and/or genotype of cell or animal contacted with the drug. In some embodiments, performing an assay includes determining lot-to-lot variability for a drug (In some embodiments, performing an assay includes determining the differences between the effects on a cell or animal described herein contacted with the drug administered and a control cell or animal (e.g., expressing a wildtype TDP-43).

Exemplary parameters that may be measured in non-human animals (or in and/or using cells isolated therefrom) for assessing the pharmacokinetic properties of a drug include, but are not limited to, agglutination, autophagy, cell division, cell death, complement-mediated hemolysis, DNA integrity, drug-specific antibody titer, drug metabolism, gene expression arrays, metabolic activity, mitochondrial activity, oxidative stress, phagocytosis, protein biosynthesis, protein degradation, protein secretion, stress response, target tissue drug concentration, non-target tissue drug concentration, transcriptional activity, and the like.

Also provided herein is a method of identifying a candidate agent for treating, preventing and/or inhibiting TDP-43 proteinopathies and/or ALS. In a specific embodiment, the inhibitory effect of the substance is determined in vivo, by administering the agent to an animal that had, at birth, on one chromosome a mutant TARDBP gene encoding a mutant ANLS TDP-43 protein or a mutant APLD TDP-43 protein as describe here and at on the other homologous chromosome a TARDBP gene that comprises a conditional knockout mutation, but at the time of the administration of the agent, has on one chromosome a mutant TARDBP gene encoding a mutant ANLS TDP-43 protein or a mutant APLD TDP-43 protein as describe here and at on the other homologous chromosome a TARDBP gene comprising a knockout mutation due to the condition (e.g., injection of Cre recombinase) having been met and wherein the animal develops ALS-like symptoms after induction of the knockout mutation.

The animals may be administered with the agent to be tested by any convenient route, for example by systemic injection, pumps for long-term exposure, or direct intracerebral injection. These animals may be included in a behavior study, so as to determine the effect of the substance on the behavior, e.g., motor behavior, of the animals compared to appropriate control animals that did not receive the agent. A biopsy or anatomical evaluation of animal spinal cord, muscle and/or brain tissue may also be performed, and/or a sample of blood or CSF may be collected.

Analysis of the motor impairment may be conducted using rotarod testing, open field locomotor testing, and catwalk testing. During catwalk testing, subjects walk across an illuminated glass platform while a video camera records from below. Gait related parameters-such as stride pattern, individual paw swing speed, stance duration, and pressure, etc., may be reported for each animal. This test may be used to phenotype transgenic strains of mice and evaluate novel chemical entities for their effect on motor performance. CatWalk XT may be a system for quantitative assessment of footfalls and gait in rats and mice, e.g., to evaluate the locomotor ability of rodents in almost any kind of experimental model of central nervous, peripheral nervous, muscular, or skeletal abnormality.

Upper motor neuron impairment presents as spasticity (i.e., rigidity), increased reflexes, tremor, bradykinesia, and Babinski signs. Lower motor neuron impairment presents as muscle weakness, wasting, clasping, curling and dragging of feet, and fasciculations. Bulbar impairment presents as difficulty swallowing, slurring and tongue fasciculations. Table 1 provides the scoring methodology related to motor impairment, tremor and rigidity of animals during testing. Assessment of overall motor function was performed using blinded subjective scoring assays, and all data is reported as mean+/−SEM.

TABLE 1

| | 0 | 1 | 2 | 3 |
|---|---|---|---|---|
| Motor | no | clasping | clasping + | paralysis |
| impairment | phenotype | | dragging/curling | |
| | | | toes | |
| Tremor | none | mild | moderate | severe |
| Rigidity | none | mild | moderate | severe |

a. Methods for Introducing Recombinase into a Cell or Animal

Various methods and compositions are provided herein to allow for introduction of a recombinase, including nucleotides comprising a sequence encoding a recombinase, into a cell or animal. Methods for introducing nucleotides comprising a sequence encoding a recombinase into various cell types are known and include, for example, stable transfection methods, transient transfection methods, and virus-mediated methods.

Transfection protocols as well as protocols for introducing nucleotides comprising a sequence encoding a recombinase into cells may vary. Non-limiting transfection methods include chemical-based transfection methods using liposomes; nanoparticles; calcium phosphate (Graham et at. (1973) Virology 52 (2): 456-467, Bacchetti et al. (1977) Proc. Natl. Acad. Sci. U.S.A. 74 (4): 1590-1594, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, Sono-poration, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleotides comprising a sequence encoding a recombinase into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of nucleotides comprising a sequence encoding a recombinase into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Methods for carrying out microinjection are well known. See, e.g., Nagy et al. (Nagy A, Gertsenstein M, Vintersten K, Behringer R., 2003, Manipulating the Mouse Embryo, Cold Spring Harbor, New York: Cold Spring Harbor Laboratory Press); see also Meyer et al. (2010) Proc. Natl. Acad. Sci. USA 107:15022-15026 and Meyer et al. (2012) Proc. Natl. Acad. Sci. USA 109:9354-9359.

Other methods for introducing nucleotides comprising a sequence encoding a recombinase into a cell can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, oligonucleotides can be introduced into a cell or non-human animal in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule.

Introduction of nucleotides comprising a sequence encoding a recombinase into a cell or animal can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression. Exemplary viral titers (e.g., AAV titers) include 10¹², 10¹³, 10¹⁴, 10¹⁵, and 10¹⁶ vector genomes/mL.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediated AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Scrotypes for heart tissue include AAV1, AAV8, and AAV9. Scrotypes for kidney tissue include AAV2. Serotypes for lung tissue include AAV4, AAV5, AAV6, and AAV9. Serotypes for pancreas tissue include AAV8. Serotypes for photoreceptor cells include AAV2, AAV5, and AAV8. Serotypes for retinal pigment epithelium tissue include AAV1, AAV2, AAV4, AAV5, and AAV8. Serotypes for skeletal muscle tissue include AAV1, AAV6, AAV7, AAV8, and AAV9. Serotypes for liver tissue include AAV7, AAV8, and AAV9, and particularly AAV8. Several synthetic AAVs that have been developed to improve transduction of the CNS. A variant, AAV-PHP.B, which can transduce the CNS much more efficiently than AAV9. Other variants, e.g., AAV-PHP.A, AAV-PHP.B, and AAV-PHP.eB also transduce the CNS in an animal. The variants AAV9-PHP.B and PHP.eB have been reported to allow for significant transduction of the CNS following intravenous infusion, but other delivery methods may be used, such as intrathecal injection, intraperitoneal injection, or intracerebroventricular injection.

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

Introduction of nucleotides comprising a sequence encoding a recombinase can also be accomplished by lipid nanoparticle (LNP)-mediated delivery. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more oligonucleotides for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1, herein incorporated by reference in its entirety for all purposes.

Administration (e.g., of nucleotides comprising a sequence encoding a recombinase, AAV, etc.) in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically.

If the cells are in vivo (e.g., in an animal), administration to the animal can be by any suitable means. For example, administration can include parenteral routes of administration, such as intraperitoneal, intravenous, and subcutaneous. Parenteral administration means administration through injection or infusion. Parenteral administration includes, for example, subcutaneous administration, intravenous administration, intramuscular administration, intraarterial administration, intraperitoneal administration, or intracranial administration (e.g., intrathecal or intracerebroventricular administration).

In some methods, administration is by a means such that the reagent being introduced reaches neurons or the nervous system. This can be achieved, for example, by peripheral delivery or by direct delivery to the nervous system. See, e.g., Evers et al. (2015) Adv. Drug Deliv. Res. 87:90-103, herein incorporated by reference in its entirety for all purposes.

For reagents (e.g., antisense oligonucleotides) to reach the nervous system, they first have to cross the vascular barrier, made up of the blood brain barrier or the blood-spinal cord barrier. One mechanism that can be used to cross the vascular barrier is receptor-mediated endocytosis. Another mechanism that can be used is cell-penetrating peptide (CPP)-based delivery systems. Different CPPs use distinct cellular translocation pathways, which depend on cell types and cargos. For example, systemically delivered antisense oligonucleotides tagged with arginine-rich CPPs are able to cross the blood brain barrier. Another delivery mechanism that can be used is exosomes, which are extracellular vesicles known to mediate communication between cells through transfer of proteins and nucleic acids. For example, IV injection of exosomes transduced with short viral peptides derived from rabies virus glycoprotein (RVG) can result in crossing of the blood brain barrier and delivery to the brain.

Techniques are also available that bypass the vascular barriers through direct infusion into the cerebrospinal fluid. For example, reagents (e.g., antisense oligonucleotides) can be infused intracerebroventricularly (ICV), after which the reagents (e.g., antisense oligonucleotides) would have to pass the ependymal cell layer that lines the ventricular system to enter the parenchyma. Intrathecal (IT) delivery means delivery of the reagents (e.g., antisense oligonucleotides) into the subarachnoid space of the spinal cord. From here, reagents (e.g., antisense oligonucleotides) will have to pass the pia mater to enter the parenchyma. Reagents (e.g., antisense oligonucleotides) can be delivered ICT or IT through an outlet catheter that is connected to an implanted reservoir. Drugs can be injected into the reservoir and delivered directly to the CSF. Intranasal administration is an alternative route of delivery that can be used.

The scope of the present invention is defined by the claims appended hereto and is not limited by particular embodiments described herein; those skilled in the art, reading the present disclosure, will be aware of various modifications that may be equivalent to such described embodiments, or otherwise within the scope of the claims. In general, terminology is in accordance with its understood meaning in the art, unless clearly indicated otherwise. References cited within this specification, or relevant portions thereof, are incorporated herein by reference in their entireties.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an” in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification.

“Control” includes the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. A “control” also includes a “control animal.” A “control animal” may have a modification as described herein, a modification that is different as described herein, or no modification (i.e., a wild type animal). In one experiment, a “test” (i.e., a variable being tested) is applied. In a second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

“Determining”, “measuring”, “evaluating”, “assessing”, “assaying” and “analyzing” includes any form of measurement and includes determining if an element is present or not. These terms include both quantitative and/or qualitative determinations. Assaying may be relative or absolute. “Assaying for the presence of” can be determining the amount of something present and/or determining whether or not it is present or absent.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure. Unless otherwise specified, any domain referred to herein refers to a TDP-43 domain.

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous” refers to a location, nucleic acid or amino acid sequence that is found or occurs naturally within a cell or animal. For example, an endogenous TARDBP sequence of a non-human animal refers to a wildtype TARDBP sequence that naturally occurs at the endogenous TARDBP locus in the non-human animal.

The term “locus” refers to a specific location of a gene (or significant sequence), DNA sequence, polypeptide-encoding sequence, or position on a chromosome of the genome of an organism. For example, a “TARDBP locus” may refer to the specific location of a TARDBP gene, TARDBP DNA sequence, TARDBP 2-encoding sequence, or TARDBP position on a chromosome of the genome of an organism that has been identified as to where such a sequence resides. A “TARDBP locus” may comprise a regulatory element of a TARDBP gene, including, for example, an enhancer, a promoter, 5′ and/or 3′ untranslated region (UTR), or a combination thereof.

The term “gene” refers to a DNA sequence in a chromosome that codes for a product (e.g., an RNA product and/or a polypeptide product) and includes the coding region interrupted with non-coding introns and sequence located adjacent to the coding region on both the 5′ and 3′ ends such that the gene corresponds to the full-length mRNA (including the 5′ and 3′ untranslated sequences). Other non-coding sequences of a gene include regulatory sequences (e.g., promoters, enhancers, and transcription factor binding sites), polyadenylation signals, internal ribosome entry sites, silencers, insulating sequence, and matrix attachment regions. These sequences may be close to the coding region of the gene (e.g., within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles, each at an endogenous locus of a homologous chromosome. Each pair of alleles represents the genotype of a specific genetic locus. Genotypes are described as homozygous if there are two identical alleles at a particular locus and as heterozygous if the two alleles differ.

“Operably linked” includes a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” includes polynucleotide sequences, which are necessary to affect the expression and processing of coding sequences to which they are ligated. “Expression control sequences” include: appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site and transcription termination sequence, while in eukaryotes typically such control sequences include promoters and transcription termination sequence. The term “control sequences” is intended to include components whose presence is essential for expression and processing, and can also include additional components whose presence is advantageous, for example, leader sequences and fusion partner sequences.

“Phenotype” includes a trait, or to a class or set of traits displayed by a cell or organism. In some embodiments, a particular phenotype may correlate with a particular allele or genotype. In some embodiments, a phenotype may be discrete; in some embodiments, a phenotype may be continuous. A phenotype may comprise viability or cellular fitness of a cell. A phenotype may comprise the expression levels, cellular localization and/or solubility/stability profile of a protein, e.g., a mutant TDP-43 polypeptide, each of which phenotypes may be determined using well-known methods such as Western Blot analysis, fluorescent in situ hybridization, qualitative RT-PCR, etc.

“Motor neurons” or “MNs”, as used herein, refer to neurons that innervate muscle fibers and are distinguished from other cell types by the selective expression of choline acetyltransferase (ChAT). “Alpha motor neurons” or “α-MNs”, as used herein, refer to motor neurons that innervate the skeletal muscle fibers that generate force (i.e., extrafusal muscle fibers). Alpha motor neurons can be distinguished from gamma motor neurons by the selective expression of NeuN. “Gamma motor neurons” or “γ-MNs”, as used herein, refer to motor neurons that innervate the muscle spindle to modulate stretch and finer motor control (i.e., intrafusal muscle fibers). Gamma motor neurons can be distinguished from alpha motor neurons by the selective expression of Err3 and GFRal. In general, α-MNs comprise the majority of motor neurons in the spinal cord (˜70-75%), while γ-MNs comprise around 25-30%. When quantifying total MNs, α-MNs, and γ-MNs, it is possible to co-stain for both ChAT (to mark all MNs) and NeuN (to distinguish alpha vs gamma), and count the number of ChAT+ MNs that are either NeuN+((α-MNs) or NeuN- (γ-MNs).

“Onset”, as used herein, refers to the earliest emergence of one or more disease-associated phenotype, e.g., symptom, in a population of subjects or in an individual subject. For example, onset of a TDP-43 proteinopathy may be characterized by the emergence of one or more motor dysfunction phenotype, e.g., hindlimb clasping, hyperactivity, etc., or one or more disease-associated symptom, e.g., weight loss, in a subject. In some embodiments, onset of symptoms for a TDP-43 disease model comprises a subject exhibiting one or more TDP-43 proteinopathy characteristics. In some embodiments, onset occurs in as little as one week, two weeks, three weeks, four weeks, five weeks, six weeks, seven weeks, eight weeks, nine weeks, ten weeks, eleven weeks, twelve weeks, five months, six months, or seven months after birth. In some embodiments, onset occurs in as little as one week, two weeks, three weeks, four weeks, five weeks, six weeks, seven weeks, eight weeks, nine weeks, ten weeks, eleven weeks, twelve weeks, five months, six months, or seven months after induction of a disease model, e.g., administration of a recombinase that recognizes a site-specific recombinase recognition sequence to create a knockout (e.g., TARDBP) gene from a conditional knockout (e.g., TARDBP) gene.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Reference” includes a standard or control agent, cell, animal, cohort, individual, population, sample, sequence or value against which an agent, cell, animal, cohort, individual, population, sample, sequence or value of interest is compared. In some embodiments, a reference agent, cell, animal, cohort, individual, population, sample, sequence or value is tested and/or determined substantially simultaneously with the testing or determination of the agent, cell, animal, cohort, individual, population, sample, sequence or value of interest. In some embodiments, a reference agent, cell, animal, cohort, individual, population, sample, sequence or value is a historical reference, optionally embodied in a tangible medium. In some embodiments, a reference may refer to a control. A “reference” also includes a “reference cell”. A “reference cell” may have a modification as described herein, a modification that is different as described herein or no modification (i.e., a wild type cell). Typically, as would be understood by those skilled in the art, a reference agent, cell, animal, cohort, individual, population, sample, sequence or value is determined or characterized under conditions comparable to those utilized to determine or characterize the agent, animal (e.g., a mammal), cohort, individual, population, sample, sequence or value of interest.

The term “variant” refers to a nucleotide sequence that differs from a reference nucleotide sequence (e.g., by one nucleotide) or a protein sequence that differs from a reference amino acid sequence (e.g., by one amino acid), but that retain the biological function of the reference sequence. In some embodiments, variants differ from the reference sequence due to degeneracy of the genetic code and/or a conservative codon/amino acid substitution.

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized in Table 2 below.

TABLE 2
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

The term “in vitro” includes artificial environments and to processes or reactions that occur within an artificial environment (e.g., a test tube). The term “in vivo” includes natural environments (e.g., a cell or organism or body) and to processes or reactions that occur within a natural environment. The term “ex vivo” includes cells that have been removed from the body of an individual and to processes or reactions that occur within such cells.

Table 3 provides a brief description of the of the sequences provided in the

TABLE 3

| SEQ ID NO | DESCRIPTION |
|---|---|
| 1 | NP_663531 - Wildtype mouse TDP-43 (Protein) |
| 2 | NM_145556.4 - Wildtype mouse TARDBP coding sequence (DNA) |
| 3 | NP_001011979 - Wildtype rat TDP-43 (Protein) |
| 4 | NM_001011979.2 - Wildtype rat TARDBP coding sequence (DNA) |
| 5 | NP_031401.1 - Wildtype human TDP-43 (Protein) |
| 6 | NM_007375.3 - Wildtype human TARDBP coding sequence (DNA) |
| 7 | RRM1 RNP2 consensus sequence (Protein) |
| 8 | RRM1 RNP1 consensus sequence (Protein) |
| 9 | RRM2 RNP2 consensus sequence (Protein) |
| 10 | RRM2 RNP1 consensus sequence (Protein) |
| 11 | Cre recombinase (Protein) |
| 12 | CAG promoter sequence (DNA) |
| 13 | hSyn promoter sequence (DNA) |
| 14 | mCherry encoding sequence (DNA) |
| 15 | polyA signal (DNA) |
| 16 | AAV 5′ ITR |
| 17 | AAV 3′ ITR |
| 18 | AAV-PHP.eB-hSyn-Cre |
| 19 | AAV-PHP.eB-CAG-Cre |
| 20 | Sortilin1 E16/19 forward primer |
| 21 | Sortilin1 E16/19 reverse primer |
| 22 | Dnajc5 E3/5 forward primer |
| 23 | Dnajc5 E3/5 reverse primer |
| 24 | Poldip3 E2/4 forward primer |
| 25 | Poldip3 E2/4 reverse primer |
| 26 | Adnp2 E2/3 forward primer |
| 27 | Adnp2 E2/3 reverse primer |
| 28 | Caskin1 E14/17 forward primer |
| 29 | Caskin1 E14/17 reverse primer |
| 30 | Tsn E4/6 forward primer |
| 31 | Tsn E4/6 reverse primer |
| 32 | GAPDH E3/5 forward primer |
| 33 | GAPDH E3/5 reverse primer |
| 34 | Cre recombinase (DNA) |

### EXAMPLES

The following examples are provided for illustrative purposes only and are not intended to limit the scope of the invention.

### Example 1: Generation of Embryonic Stem Cells and Mice Expressing a Mutated TARDBP Gene

Since TDP-43 is essential for viability, embryonic stem (ES) cells comprising a conditional knockout on a first endogenous TDP-43 allele and a mutation on the other second endogenous TDP-43 allele may be generated such that wildtype TDP-43 from the first endogenous allele sustains viability of the ES cell and any animal developed therefrom until activation of the condition, after which activation the effects of the mutant TDP-43 polypeptide expressed from the second allele may be ascertained.

A conditional allele may be designed based on previously published work that shows deletion of TDP-43 exon 3 produces no functional protein. Chiang et al. (2010) Proc Natl Acad Sci USA 107:16320-324. Exon 3 of the endogenous mouse TARDBP gene may be floxed with loxP sites. See, FIG. 3A. After Cre-mediated recombination, deletion of the genomic coordinates chr4:147995844-147996841 will be effected.

To determine whether a neuromuscular TDP-43 proteinopathy phenotype could be produced in mice, mouse embryonic stem (ES) cells comprising the floxed exon 3 conditional knockout mutation on one chromosome were further modified with a mutated TARDBP gene on the other homologous chromosome. ES cells were modified to comprise: (i) at an endogenous TARDBP locus, a TARDBP gene comprising a conditional knockout mutation, see FIG. 3A, and (ii) at the other TARDBP locus on a homologous chromosome, a mutated TARDBP gene that encodes a mutant TDP-43 polypeptide in which the nuclear localization signal (NLS) or the prion like domain (PLD) was either altered in ways predicted to abolish their functions or deleted, respectively, see, FIG. 3B.

As a control, mouse ES cells modified with the conditional knockout mutation on one allele and a wildtype TARDBP gene on the other allele were also created.

To create embryos or animals that express a mutant TDP-43 protein lacking a functional NLS or PLD domain, the VelociMouse® method (Dechiara, T.M., (2009), Methods Mol Biol 530:311-324; Poueymirou et al. (2007), Nat. Biotechnol. 25:91-99) was used, in which targeted ES cells comprising
(i) at an endogenous TARDBP locus, a TARDBP gene comprising a conditional knockout floxed exon 3 (flEx3) mutation, a knockout TARDBP gene comprising a Cre-mediated deletion of the floxed exon 3 (ΔEx3), a mutated TARDBP gene comprising knockout mutations in the NLS (ΔNLS), or a mutated TARDBP gene comprising a deletion of the prion like domain (ΔPLD), or a wildtype TARDBP gene (WT), and
(ii) at the other TARDBP locus on a homologous chromosome, a wildtype (WT) TARDBP gene, a TARDBP gene comprising a conditional knockout floxed exon 3 (flEx3) mutation, or a knockout TARDBP gene comprising a Cre-mediated deletion of the entire coding sequence (˜)
   were injected into uncompacted 8-cell stage Swiss Webster embryos. The viability of embryos after fertilization was examined and the ability to produce live-born F0 generation mice was assessed.

Consistent with prior experiments, embryos lacking a functional TDP-43 protein (TDP-43^{ΔEx3/-}) were not viable and did not survive beyond the E3.5 stage. Similarly, embryos expressing only a TDP-43 protein lacking a functional NLS (TDP-43^{ΔNLS/ΔEx3}) or only a TDP-43 protein lacking a functional PLD (TDP-43^{ΔPLD/ΔEx3}) were not viable, although such embryos survived longer. See, FIG. 4. Expression of a wildtype TDP-43 protein from one allele of the TARDPB locus rescued embryos expressing from the other allele on a homologous chromosome either a TDP-43 protein lacking a functional NLS (TDP-43^{ΔNLS/WT}, TDP-43^{ΔNLS/flEx3}) or a TDP-43 protein lacking a functional PLD (TDP-43^{ΔPLD/WT}, TDP-43^{ΔPLD/flEx3}). See, FIG. 4.

Live-born F0 generation mice were successfully produced from 8-cell stage Swiss Webster embryos injected with ES cells comprising
(i) at an endogenous TARDBP locus, a knockout mutation in the NLS (ΔNLS) or a deletion of the prion like domain (ΔPLD), see, FIG. 3B, and
(iii) at the other TARDBP locus on a homologous chromosome, a wildtype gene (WT), or a TARDBP gene comprising cre-mediated deletion of a floxed exon 3 (−). See, FIG. 3A
   Mice homozygous for the conditional knockout allele (TDP-43^{flEx3/flEx3}), as well as mice that have one WT allele paired with the conditional knockout allele (TDP-43^{flEx3/WT}) were used as controls.

### Example 2: Producing a neuromuscular pathologies in mice expressing only a mutated TARDBP gene

To determine whether mutant TDP-43 proteins mediate the neuromuscular pathologies, mice according to Example 1 may be used since the mice harbor at an endogenous TARDBP locus, an exon 3 floxed conditional knockout (cKO) allele (“loxP-Ex3-loxP”) that undergoes Cre-mediated recombination to produce a ΔEx3 knockout allele when in the presence of Cre, and at the other TARDBP locus on a homologous chromosome, either the ΔNLS or ΔPLD mutants (TDP-43^{flEx3/ΔNLS} or TDP-43^{flEx3/ΔPLD}, respectively).

### Cre-Induced Neuromuscular Phenotypes in Mice

To determine whether the neuromuscular phenotype seen in heterozygous adult ΔNLS/WT and ΔPLD/WT mice (see, e.g., WO 2020/264339A4, incorporated herein in its entirety by reference) may be exacerbated, AAV-mediated delivery of Cre recombinase was used to remove the WT allele leaving only the mutant forms of TDP-43 in cells transduced by the virus. For these experiments, mice harboring an exon 3 floxed conditional knockout (cKO) allele (“flEx3”) that undergoes Cre-mediated recombination to produce a ΔEx3 knockout allele when in the presence of Cre were used. The flEx3 conditional allele was paired with either the ΔNLS or ΔPLD domain mutants (TDP-43^{flEx3/ΔNLS} or TDP-43^{flEx3/ΔPLD}). As a control, mice that have one wildtype allele paired with the conditional allele (TDP-43^{flEx3/WT}) as well as homozygous wild type (TDP-43^{WT/WT}) mice were used.

To specifically ablate the conditional TDP-43 allele to neurons within the CNS, a PHP.eB.AAV virus expressing a Cre-2A-mCherry cassette driven by the neuron-specific human synapsin promoter (PHP.eB.AAV-SYN-Cre-2A-mCherry) was generated. Because the AAV.PHP.eB capsid has both high tropism for neurons and the ability to efficiently cross the blood-brain-barrier (BBB), high neuronal transduction can occur following intraperitoneal (i.p.) injection of the virus. Therefore, to specifically target neurons, 5 month old adult control and mutant mice (TDP-43^{flEx3/WT}, TDP-43^{flEx3/ΔNLS}, and TDP-43^{flEx3/ΔPLD}) were given 1×10¹¹ viral genomes of PHP.eB.AAV-SYN-Cre-2A-mCherry by i.p. injection. Following Cre-mediated recombination, both TDP-43^{ΔEx3/ΔNLS} and TDP-43^{ΔEx3/ΔPLD} mice show a premature death phenotype compared to either uninjected control mice (TDP-43^{WT/WT}) or injected heterozygous mice (TDP-43^{ΔEx3/WT}) (FIG. 5). Additionally, injected mice harboring the domain mutant versions of TDP-43 show a loss in body weight following Cre injection (FIG. 6). Specifically, TDP-43^{ΔEx3/ΔPLD} mice display an approximately 25% reduction in body weight during the first two months post-injection, which is then maintained for the following 4-5 months followed by a further decline. TDP-43^{ΔEx3/ΔNLS} display an even greater body weight reduction of approximately 50% during the first two months post-injection that appears to be maintained for several months before an additional decline (FIG. 6). In addition to reduced survival and body weight, both TDP-43^{ΔEx3/ΔNLS} and TDP-43^{ΔEx3/ΔPLD} mice display clear motor deficits: TDP-43^{ΔEx3/ΔPLD} mice show defects in their hindlimb movement and function that progresses to hindlimb paralysis; TDP-43^{ΔEx3/ΔNLS} mice display abnormal and erratic movements that progress with age and lead to paralysis as well.

Heterozygous adult ΔNLS/WT and ΔPLD/WT mice show mislocalization of TDP-43 in motor neurons when immunostaining with antibodies again TDP-43 in the spinal cord. WO 2020/264339A4, incorporated herein in its entirety by reference. Further, ΔPLD/WT mice show the ΔPLD protein to be more cytoplasmic than nuclear, which causes the WT protein to mislocalize from the nucleus to the cytoplasm and accumulate in cytoplasmic aggregates. WO 2020/264339A4, incorporated herein in its entirety by reference. Alternatively, the ΔNLS/WT mice shown an increase in both nuclear and cytoplasmic TDP-43 that also aggregates in the cytoplasm. WO 2020/264339A4, incorporated herein in its entirety by reference. Due to limitations in detection tools, the ΔNLS and WT proteins are unable to be distinguished by immunohistochemistry, although an antibody raised against the C-terminus of TDP-43 (Proteintech cat #12892-1-AP) that is blind to the ΔPLD form can be used to detect the wildtype TDP-43 protein in this genetic context. Spinal cord sections from TDP-43^{ΔEx3/ΔNLS} and TDP-43^{ΔEx3/ΔPLD} mice 7 to 8 months post-injection immunostained with an antibody against the C-terminus of TDP-43 results in the detection of large cytoplasmic aggregates within motor neurons (FIG. 7, top panels). Since, in the context of the TDP-43^{ΔEx3/ΔPLD} mice where the C-terminal antibody does not recognize the ΔPLD form only the WT, this demonstrates that the aggregates that remain contain WT protein that have persisted for at least 7 to 8 months after Cre-mediated removal of the WT allele. These aggregates were also detected in TDP-43^{ΔEx3/ΔNLS} mice (FIG. 7, top panels). In addition to these long-persisting aggregates, the total number of motor neurons appeared to be reduced in mutant mice compared to controls (FIG. 7, bottom panels).

To determine in a more quantitative manner whether the exacerbation of motor behavioral phenotypes of injected mice and TDP-43 aggregation correlated with cellular defects in the motor system, the survival and function of two key components of the motor system: motor neurons (MNs) and the neuromuscular junction (NMJ), were analyzed. To first determine if motor neuron death was occurring, the average number of lateral motor column (LMC) motor neurons in the lumbar L4-L6 region of the spinal cord using choline acetyltransferase (ChAT) as a marker of motor neurons was determined.

Compared to control mice, both TDP-43^{ΔEx3/ΔNLS} and TDP-43^{ΔEx3/ΔPLD} mice showed a greater than 30% reduction in the number of LMC MNs at 7 to 8 months post-injection (FIG. 8A). In addition to clear MN death, both mutant lines displayed significant denervation of both tibialis anterior (FIG. 8B) and soleus hind limb muscles (FIG. 8C), supporting a loss of motor system connectivity in this model.

### Cre-Induced Neuromuscular Phenotypes in Neonates

To determine whether an accelerated neuromuscular phenotype could be produced in mice, mice were forced to express either the ΔNLS or ΔPLD mutants of TDP-43 as the only form of the protein in a subset of CNS cells at an early post-natal timepoint. This was accomplished by intracerebroventricular injection of 5×10¹⁰ viral genomes comprising Cre recombinase driven by either a ubiquitous CAG promoter (PHP.eB.AAV-CAG-Cre-2A-mCherry) or a neuron-specific human synapsin promoter (PHP.eB.AAV-SYN-Cre-2A-mCherry) into P0 pups.

When P0 newborn pups were forced to express only ΔNLS or ΔPLD mutants ubiquitously or in certain CNS cells, mice homozygous for the TARDBP gene comprising the conditional knockout mutation (TDP-43^{ΔEx3/ΔEx3}) died between 4 and 5 weeks after Cre delivery (FIGS. 9A and 9B), while TDP-43^{ΔEx3/WT} heterozygous mice survive much longer. Interestingly, both TDP-43^{ΔEx3/ΔNLS} and TDP-43^{ΔEx3/ΔPLD} mice are able to survive longer than TDP-43^{ΔEx3/ΔEx3} when Cre is driven ubiquitously through the CAG promoter, suggesting some aspect of functional compensation of both ΔNLS and ΔPLD alleles, however both genotypes die prematurely between 9-12 weeks of age (FIG. 9A). Median survival times were as follows: TDP-43^{ΔEx3/ΔEx3}: 4 weeks; TDP-43^{ΔEx3/ΔNLS}: 10.86 weeks; TDP-43^{ΔEx3/ΔPLD}: 9.43 weeks; TDP-43^{ΔEx3/WT}: 17.36 weeks. Moreover, TDP-43^{ΔEx3/ΔNLS} and TDP-43^{ΔEx3/ΔPLD} mice exhibited significant weight loss compared to uninjected controls at 4 weeks after birth (data not shown). Furthermore, when these same cohorts of mice were injected with AAV-Cre driven by a human synapsin promoter (SYN-Cre) to restrict expression to neurons, mice with ΔPLD as the only form of TDP-43 in neurons display enhanced survival indicating ΔPLD can retain some functionality in neurons when it is the only form of TDP-43 post-natally (FIG. 9B). Notably, the ΔNLS form of the protein remains equally as lethal in the SYN-Cre context as it does in the CAG-Cre, suggesting this domain mutant severely affects TDP-43 function in neurons (FIG. 9B). Median survival times were as follows: TDP-43^{ΔEx3/ΔEx3}: 3.93 weeks; TDP-43^{ΔEx3/ΔNLS}: 10.7 weeks; TDP-43^{ΔEx3/ΔPLD}: 25 weeks; TDP-43^{ΔEx3/WT}: 52 weeks. Additionally, no significant weight loss up to 12 weeks was observed for TDP-43^{ΔEx3/ΔNLS} mice, while a small subset (˜25%) of TDP-43^{ΔEx3/ΔPLD} mice exhibited significant weight loss, and the remainder only exhibited minor weight loss (˜15%) at 4 weeks after birth as compared to uninjected controls (data not shown).

In addition to the shortened survival phenotype, both TDP-43^{ΔEx3/ΔNLS} and TDP-43^{ΔEx3/ΔPLD} mice display severe motor behavioral phenotypes following Cre mediated deletion of exon 3 of a TARDBP gene comprising the floxed exon 3 conditional knockout mutation using CAG-Cre. By seven to ten weeks of age, designated as “late symptomatic”, nearly all TDP-43^{ΔEx3/ΔNLS} and TDP-43^{ΔEx3/ΔPLD} mice display kyphosis, hindlimb clasping, while at 6 weeks of age, designated as “early symptomatic”, the clasping phenotype is just starting to become apparent. See, FIG. 10A. FIG. 10B and FIG. 10C depict an overlay of the “early symptomatic” and “late symptomatic” windows on the first 18 weeks of the survival curves depicted in FIG. 9A and FIG. 9B, respectively. Specifically, as early as 5 weeks TDP-43^{ΔEx3/ΔPLD} mice exhibit onset of clasping symptoms, as early as 7 weeks they begin to exhibit onset of kyphosis, and by 7-10 weeks of age they exhibit kyphosis and strong hindlimb clasping that can progress to hindlimb paralysis and an inability to right themselves (data not shown). Alternatively, CAG-Cre injected TDP-43^{ΔEx3/ΔNLS} mice display a distinct and noticeable hyperactivity phenotype early (as early as 5 weeks after birth) that is followed by onset of clasping and kyphosis as early as 6 weeks after birth, and culminating in uncoordinated and ataxic movements, head wobbling, an inability to right themselves and eventual paralysis at about 12 weeks after birth. See, FIG. 10A. Interestingly, SYN-Cre injected ΔNLS mice demonstrate a very similar hyperactivity phenotype as with CAG-Cre (as early as 5 weeks after birth) but no sustained clasping phenotypes up to 12 weeks old, while SYN-Cre injected ΔPLD mice show a range of phenotypes in which a subset (˜40%) show onset of hindlimb clasping as early as 9 weeks after birth, a subset (˜40-50%) develop kyphosis as early as 7 weeks after birth, and a subset develop striking hind limb paresis and muscle wasting, while a contrasting subset appear nearly normal with no overt phenotypes until much later (>4 months) (data not shown).

To determine whether these striking motor behavioral phenotypes could be caused by defects in the motor system at the cellular level, neuromuscular junction (NMJ) denervation in a panel of skeletal muscles including forelimb (triceps and biceps), hindlimb (tibialis anterior, gastrocnemius, and soleus), and intercostal muscles was determined. CAG-Cre injected mice were analyzed at two timepoints: an “early symptomatic” point at 6 weeks of age, when the majority of mice had not yet started to display strong motor issues, as well as a “late symptomatic” point at 10 weeks, when most mice had clear motor behavioral issues. Uninjected mice served as controls in this analysis. Progressive NMJ denervation was observed between early symptomatic (6 weeks) and late symptomatic (10 weeks) time points in TDP-43^{ΔEx3/ΔPLD} and TDP-43^{ΔEx3/ΔNLS} mice, but not in controls. See, FIG. 11. While mice at 6 weeks displayed minimal denervation at 6 weeks, this significantly progressed by 10 weeks across all muscles tested. See, FIG. 11. Interestingly, the hindlimb and forelimb muscles show greater denervation than intercostal muscles, which is consistent with intercostal muscles denervating at much later stages than limb muscles in ALS disease. See, FIG. 11. In the SYN-Cre cohort, preliminary analysis suggests a moderate and similar amount of NMJ denervation in ΔPLD animals at 10 weeks post-Cre (˜15-20%; FIG. 13A).

Since TDP-43^{ΔEx3/ΔNLS} and TDP-43^{ΔEx3/ΔPLD} mice displayed clear motor axon retraction and denervation of motor neurons from skeletal muscle, the average number of lateral motor column (LMC) motor neurons in the lumbar L4-L6 region of the spinal cord using ChAT as a marker of motor neurons was examined to determine if motor neuron death was occurring. Within these pools of motor neurons, the number of alpha versus gamma motor neurons was determined by the absence (gamma) or presence (alpha) of NeuN among ChAT-positive motor neurons (see FIG. 12, right panel). Both TDP-43^{ΔEx3/ΔNLS} and TDP-43^{ΔEx3/ΔPLD} mice injected with CAG-Cre showed a reduction in total motor neuron number compared to TDP-43^{ΔEx3/WT} controls (see FIG. 12, left panel). TDP-43^{ΔEx3/ΔNLS} mice seem to have a greater reduction in motor neuron number compared to TDP-43^{ΔEx3/ΔPLD}, which is consistent with TDP-43^{ΔEx3/ΔNLS} mice having slightly greater NMJ denervation. Notably, the large, NeuN+ (green), ChAT+ (blue) alpha motor neurons appear to be selectively reduced in mutant spinal cords, while the small, NeuN−, ChAT+ gamma motor neurons remain unaffected. (see FIG. 12, left panel). The amount of motor neuron loss in TDP-43^{ΔEx3/ΔNLS} mice appeared consistent between animals treated with Cre driven by either the CAG or SYN promoters. Interestingly, the reduction in motor neuron number in both TDP-43^{ΔEx3/ΔNLS} and TDP-43^{ΔEx3/ΔPLD} mice appears to be driven by the loss of alpha motor neurons specifically, as gamma motor neurons showed no change. (see FIG. 12, left panel). This is consistent with gamma motor neurons being resistant to degeneration in multiple mouse models of ALS (e.g., SOD1, FUS and TDP-43 models) as well as in human patients (Kawamura et al. J Neuropathol Exp Neurol 1981, Sobue et al. Acta Neuropathol 1981, Conradi et al. Brain Res Bull 1993, Wetts and Vaughn Exp Neurol 1996, Mohajeri et al. Exp Neurol 1998, Lalancette-Hebert et al. PNAS 2016, each of which reference is hereby incorporated in its entirety by reference).

Moreover, as shown in FIG. 13B, motor neurons show a slight reduction in total number in ΔPLD and ΔNLS mice treated with Cre-expressing virus compared to control animals (uninjected or WT/ΔEx3-injected mice). While there may be some TDP43-independent reduction in motor neuron number in the injected controls (WT/ΔEx3) compared to uninjected controls caused by very high Cre expression, a small additional reduction is observed in TDP-43 domain mutant mice indicating an additional loss due to TDP-43 dysfunction. The relevance of this mild loss is supported by the observation that the alpha motor neurons are selectively being lost, while gamma motor neurons remain preserved, which is a hallmark ALS disease phenotype. It is noteworthy that this analysis is done 10 weeks after Cre-injection, which is a short window of time to allow for TDP-43 ablation to lead to motor neuron dysfunction and death. When compared to WT/ΔEx3+CAG-Cre mice as the baseline control, ΔPLD+CAG-Cre mice show no loss in total MNs and a 9% loss in α-MNs; ΔNLS+CAG-Cre show a 6% loss in total MNs and a 25% loss in c-MNs, and ΔNLS+SYN-Cre show a 6% loss in total MNs and a 22% loss in α-MNs.

In general, the distinct phenotypes observed in ΔNLS and ΔPLD mice are more severe and have a quicker onset with the CAG-Cre-driven removal of the conditional WT allele than the SYN-Cre cohort, which can trigger similar phenotypes but not uniformly and typically not until later in age.

The most well-characterized function of TDP-43 is in regulating RNA splicing (see FIG. 14). Specifically, TDP-43 binds to intronic sequences to suppress cryptic exons from being aberrantly included in mRNA transcripts, and can also control alternative splicing events. To assay for TDP-43 function, semi-quantitative RT-PCR for specific splicing events in Adnp2, Dnajc5, Poldip3, Tsn, and Sortilini, determined to be TDP-43-dependent, was performed at 12 weeks of age. While control animals without CAG-Cre expression display normal transcript processing (see FIG. 15, lanes 1 and 3). homozygous removal of the conditional allele (TDP-43^{ΔEx3/ΔEx3} FIG. 15, lane 2) and heterozygous removal of the conditional allele in the presence of a mutant allele (FIG. 15, lanes 4-6) triggers strong loss-of-function of TDP-43 in regulating the splicing of all transcripts tested. Both TDP-43^{ΔEx3/ΔNLS} and TDP-43^{ΔEx3/ΔPLD} mice displayed disrupted transcript splicing clearly implicating a loss of TDP-43 function when these mutations are the only form of TDP-43. Interestingly, TDP-43^{ΔEx3/ΔNLS} mice appeared to show less functionality than TDP-43^{ΔEx3/ΔPLD}, which is consistent with TDP-43^{ΔEx3/ΔPLD} mice demonstrating slightly milder denervation and motor neuron loss phenotypes. Importantly, TDP-43^{ΔEx3/WT} mice treated with CAG-Cre or SYN-Cre displayed normal splicing profiles, indicating any effects of splicing is TDP-43-dependent and not caused non-specifically by Cre expression. Collectively, this data demonstrates the generation of rapid neonatal model of TDP-43-mediated neurodegeneration.

The materials and methods used to analyze the phenotype of mice expressing both only a mutant TDP-43 polypeptide lacking a functional NLS or PLD in a subset of CNS cells are described below.

### Intraperitoneal (i.p) Injection in Adult Mice

Injections were carried out using a 0.3 cc insulin syringe with a 27 gauge needle. Each mouse received a single injection containing 1×10¹¹ viral genomes in a total volume of 50 μl with PBS. Five month old mice were manually restrained and injected in the lower right quadrant of their peritoneum. Mice were closely monitored immediately post-injection for any signs of injection site bleeding and then placed back in their home cages.

### Intracerebroventricular (i.e.v.) Injection in Neonatal Mice

Newborn pups (P0/P1) were anesthetized on ice for ˜7-10 minutes until toe pinch reflexes were diminished. Pups were placed under a dissecting microscope with an attached light source. Injections were done using a 10 μL Hamilton syringe (Hamilton cat #7653-01) fitted with a custom removable needle (32 gauge, 12 degree point angle, 0.75 inches long). Pups were given a single injection into the left ventricle at a position approximately 2 mm laterally from the superior sagittal sinus and 2 mm rostrally from the transverse sinus to a depth of approximately 2 mm. For PHP.eB.AAV-Cre, each injection contains 3.5-5.0×10¹⁰ viral genomes in a volume of 5 μL diluted with PBS and containing FastGreen dye (0.03%) to visualize fluid distribution. After injection, pups were placed on a 37° C. heating pad, monitored for full recovery, and then placed back in their home cages.

### Quantification of Motor Neuron Number in Mouse Spinal Cords

Mice were perfused with ˜10 ml of 0.1M phosphate buffer (PB) followed by ˜20 ml 4% paraformaldehyde (PFA). Brain and spinal cord were kept intact within the skull and vertebral column and post-fixed in 4% PFA overnight at 4° C. followed by removal and replacement with 0.1M PB the following day. Fixed spinal cord was dissected out of the vertebral column and the specific L4-L6 segment was identified by the ventral roots and dissected out. Segments were embedded in 4% low-melt agarose and sectioned at 70 μm using a VT1000 S vibratome (Leica). Sections were blocked overnight at room temperature with 10% normal donkey serum diluted in TBS containing 0.2% Triton-X (TBS-T) and supplemented with 0.05% sodium azide. The next day, sections were incubated with primary antibody diluted in blocking solution for 2 days at room temperature. The antibodies used are as follows: anti-ChAT (Millipore cat #AB 144P), 1:200; anti-NeuN (Millipore cat #MAB377), 1:500). Following primary incubation, sections were washed six times, 30 minutes each wash with TBS-T, followed by secondary antibody incubation overnight at room temperature diluted in TBS-T. After six, 30 minute washes in TBS-T, sections were mounted to slides using Fluoromount-G (SouthernBiotech). Images were acquired using a Zeiss LSM780 confocal microscope. For quantification of motor neuron number, images were acquired using a 20× objective at 3 um steps along the entire z-axis of the section. The total number of motor neurons was determined by manually counting the ChAT+ cells in the lateral motor column located in the ventral horn. Among the ChAT+ motor neurons, the number of alpha and gamma motor neurons were determined by the absence (gamma) or presence (alpha) of NeuN staining. Motor neuron numbers are represented as the average number of motor neurons per section.

### Quantification of Neuromuscular Junction Innervation

Mice were perfused with ˜10 ml of 0.1M phosphate buffer (PB) followed by ˜20 ml 4% paraformaldehyde (PFA). Following a tissue wash in PB, muscles were dissected off the bone and cryoprotected in 30% sucrose in PB overnight at 4° C. Cryoprotected muscles were embedded in optimal cutting temperature compound (O.C.T.) and frozen at −80° C. Cryosections were cut at 30 m on a Leica cryostat onto SuperFrost Plus positively charged glass slides. Sections were blocked with 5% donkey serum in TBS containing 0.2% Triton-X (TBS-T) for 1 hour at room temperature before staining. Sections were incubated with primary antibodies diluted in blocking solution overnight at 4° C. The antibodies used to detect the pre-synapse were: anti-synaptophsyin (Invitrogen cat #PA1-1043), 1:500 and anti-neurofilament (Millipore cat #AB1987), 1:500. Following primary incubation, sections were washed three times for 10 minutes with TBS-T, followed by secondary antibody incubation, including AlexaFluor-488-conjugated alpha-bungarotoxin (BTX; ThermoFisher cat #B 13422; 1:500) to visualize the post-synaptic acetylcholine receptors (AChR), for 1 hour at room temperature. After secondary, sections were washed three times for 10 minutes and mounted with a glass coverslip using Fluoromount-G (SouthernBiotech). Sections were imaged on a Zeiss AxioScan slidescanner across the entire z-axis. To quantify NMJ innervation, images were manually scored for BTX-positive AChRs that had pre-synaptic signal present (innervated NMJ) or absent (denervated NMJ).

### RT-PCR of Splicing Events

Freshly dissected tissue was immediately placed in RNAlater and stored at −20° C. until RNA isolation. Total RNA was isolated using Trizol reagent followed by DNase treatment. For cDNA, 1 g of total RNA was used as a template for cDNA synthesis using SuperScript IV First-Strand Synthesis System (ThermoFisher cat #18091050). Reactions were carried out in a volume of 20 μl and then brought up to a final volume of 100 μl after cDNA synthesis was complete. PCR reactions were carried out using Q5 2× MasterMix (NEB) with 2 μl of cDNA template, 1 mM of each forward and reverse primer, in a total reaction volume of 25 μl. For each transcript, PCRs were first optimized to determine the cycle number that allowed for amplification that remained unsaturated and within a linear range. Reactions were run on 1.8% agarose gels in 1×TAE and bands were visualized using SybrSafe. The primers used and corresponding cycle numbers are listed below in Table 4.

TABLE 4

| Target | Fwd primer (5′ to 3′) | Rev primer (5′ to 3′) | Cycles |
|---|---|---|---|
| Sortilin1 E16/19 | GCATGAGTTAGAGTTCTGTCTG | CTTCCGCCACAGACATATTTC | 30 |
| | (SEQ ID NO: 20) | (SEQ ID NO: 21) | |
| Dnajc5 E3/5 | CTACTTCGTACTCTCCAGCTG | GATGCTGGCTGTATGACGATC | 29 |
| | (SEQ ID NO: 22) | (SEQ ID NO: 23) | |
| Poldip3 E2/4 | GAGAAGATCAGCTTGAAGAGG | CACCACAATGTCATCATCTTC | 30 |
| | (SEQ ID NO: 24) | (SEQ ID NO: 25) | |
| Adnp2 E2/3 | GCAGAATCTTGACAACATCAGG | GCTTTCTTTCCAGAAGGTTCC | 29 |
| | (SEQ ID NO: 26) | (SEQ ID NO: 27) | |
| Caskin1 E14/17 | CACCGAAGAAGCTGGAATC | CCAGGTGATATCGGTGATG | 30 |
| | (SEQ ID NO: 28) | (SEQ ID NO: 29) | |
| Tsn E4/6 | GTTTCATGAGCATTGGCGGTTC | GTAGTCTCCAGCAGTGACACTG | 30 |
| | (SEQ ID NO: 30) | (SEQ ID NO: 31) | |
| GAPDH E3/5 | ACCACCATGGAGAAGGCCGGG | CAGTGATGGCATGGACTGTGG | 26 |
| | (SEQ ID NO: 32) | (SEQ ID NO: 33) | |

What is claimed is:

1. A non-human animal comprising in its central nervous system (CNS) a plurality of cells that each comprises:
(a) a mutated TARDBP gene at one chromosome at an endogenous TARDBP locus,
wherein the mutated TARDBP gene comprises a mutation in a nuclear localization signal (NLS) encoding sequence or a prion like domain (PLD) encoding sequence such that the mutated TARDBP gene encodes a mutant TDP-43 polypeptide that lacks a functional NLS or a functional PLD, and
(b) a knockout TARDBP gene at the other homologous chromosome at an endogenous TARDBP locus,
wherein the knockout TARDBP gene comprises the wildtype TARDBP gene sequence that comprises a loss-of-function mutation.

2. The non-human animal of claim 1, wherein the knockout TARDBP gene comprises a deletion of its exon 3.

3. The non-human animal of claim 1 or claim 2, wherein the plurality of cells comprises neurons.

4. The non-human animal of any one of claims 1-3, wherein the non-human animal further comprises a second plurality of cells,
wherein each of the second plurality of cells comprises:
(a) the mutated TARDBP gene on one chromosome at an endogenous TARDBP locus, and
(b) a conditional knockout TARDBP gene at the other homologous chromosome at an endogenous TARDBP locus,
wherein the conditional knockout TARDBP gene comprises the wildtype TARDBP gene sequence with at least one exon flanked by a site-specific recombinase recognition sequence and encodes a wildtype TDP-43 protein, and
wherein recognition of the site-specific recombinase recognition sequence by a recombinase results in the deletion of at least one exon and formation of the knockout TARDBP gene.

5. The non-human animal of claim 4, wherein exon 3 of the conditional knockout TARDBP gene is flanked by the site-specific recombinase recognition sequence.

6. The non-human animal of claim 4 or claim 5, wherein the site-specific recombinase recognition sequence comprises a loxP sequence and the recombinase is Cre recombinase.

7. The non-human animal of any one of claims 1-6, wherein the non-human animal further comprises a nucleic acid comprising a sequence that encodes a recombinase,
optionally wherein the nucleic acid further comprises:
(i) a promoter sequence that drives the expression of the recombinase,
(ii) a reporter gene sequence, optionally wherein the reporter gene sequence is operably linked to the recombinase gene sequence by a poly A sequence,
(iii) an adeno-associated virus (AAV) inverted terminal repeat (ITR) sequence at the 5′ and 3′ ends of the nucleic acid, or
(iv) any combination of (i)-(iii).

8. The non-human animal of claim 7, wherein the promoter sequence comprises a CNS-tissue specific promoter sequence.

9. The non-human animal of claim 7 or claim 8, wherein the promoter sequence comprises a human synapsin promoter sequence.

10. The non-human animal of any one of claims 7-9, wherein the nucleic acid comprises a sequence set forth as SEQ ID NO:18 or SEQ ID NO:19.

11. The non-human animal of any one of claims 1-10, wherein the wildtype TARDBP gene is an endogenous wildtype TARDBP gene of the non-human animal.

12. The non-human animal of any one of claims 1-10, wherein the wildtype TARDBP gene is a wildtype human TARDBP gene.

13. The non-human animal of any one of claims 1-12, wherein the mutant TDP-43 polypeptide comprises:
(a) a point mutation of an amino acid in the NLS, or
(b) a deletion of at least a portion of the prion-like domain.

14. The non-human animal of claim 13, wherein
(a) the point mutation of an amino acid in the NLS comprises K82A K83A, R84A, K95A, K97A, K98A, or a combination thereof, and
(b) the deletion of at least a portion of the prion-like domain comprises a deletion of the amino acids at and between positions 274 and 414 of a wildtype TDP-43 polypeptide.

15. The non-human animal of any one of claims 1-14, wherein the mutant TDP-43 polypeptide comprises K82A K83A, R84A, K95A, K97A, and K98A point mutations.

16. The non-human animal of any one of claims 1-14, wherein the mutant TDP-43 polypeptide lacks the prion-like domain between and including the amino acids at positions 274 to 414 of a wildtype polypeptide.

17. The non-human animal of any one of claims 1-16, wherein the mutated TARDBP gene replaces an endogenous TARDBP gene, and
wherein the knockout TARDBP gene replaces an endogenous TARDBP gene.

18. The non-human animal of any one of claims 1-17, wherein the non-human animal is a rat.

19. The non-human animal of any one of claims 1-17, wherein the non-human animal is a mouse.

20. The non-human animal of any one of claims 1-19, wherein the non-human animal exhibits one or more of the following TDP-43 proteinopathy characteristics in comparison to a control non-human animal:
(i) weight loss
(ii) cytoplasmic aggregation of TDP-43,
(iii) decreased number of motor neurons in the spinal cord,
(iv) disruption of TDP-43 function in cryptic and alternative splicing of mRNA transcripts,
(v) denervation of neuromuscular junctions,
(vi) a motor phenotype, and/or
(vii) early lethality,
wherein each cell of the control non-human animal comprises:
(a) the mutated TARDBP gene at one chromosome at an endogenous TARDBP locus, and
(b) a wildtype TARDBP gene or a conditional knockout TARDBP gene at the other homologous chromosome at an endogenous TARDBP locus,
wherein the conditional knockout TARDBP gene comprises the wildtype TARDBP gene sequence with at least one exon flanked by a site-specific recombinase recognition sequence and encodes a wildtype TDP-43 protein, and
wherein recognition of the site-specific recombinase recognition sequence by a recombinase results in the deletion of at least one exon and formation of the knockout TARDBP gene.

21. The non-human animal of claim 20, wherein the motor phenotype comprises one or more selected from the group consisting of: hind limb clasping, kyphosis, early hyperactivity, uncoordinated/ataxic movement, head wobbling, paralysis, and inability to right.

22. The non-human animal of claim 20, wherein the decreased number of motor neurons in the spinal cord comprises a decrease in the number of alpha motor neurons, but not gamma motor neurons.

23. The non-human animal of any one of claims 1-22, wherein the non-human animal exhibits onset of the one or more TDP-43 proteinopathy characteristics by, at, and/or around four to five weeks after birth.

24. The non-human animal of claim 23, wherein the non-human animal exhibits at least two of the one or more TDP-43 proteinopathy characteristics by, at, and/or around seven to ten weeks after birth.

25. The non-human animal of any one of claims 20-24, wherein both the non-human animal and the control non-human animal are each a rat.

26. The non-human animal of any one of claims 20-24, wherein both the non-human animal and the control non-human animal are each a mouse.

27. A non-human animal cell isolated from the non-human animal of any one of claims 1-26, optionally wherein the non-human animal comprises:
(a) the mutated TARDBP gene at one chromosome at an endogenous TARDBP locus, and
(b) the knockout TARDBP gene at the other homologous chromosome at an endogenous TARDBP locus.

28. A composition comprising the non-human animal cell of claim 27.

29. A method of identifying a therapeutic candidate agent for the treatment of TDP-proteinopathy and/or an associated disease, the method comprising
(a) contacting the non-human animal of any one of claims 1-26 with the candidate agent,
(b) evaluating a phenotype and/or a biological function of TDP-43 in the non-human animal, and
(c) identifying the candidate agent that prevents or reduces the exhibition of one or more of the following TDP-43 proteinopathy characteristics in the non-human animal:
(i) weight loss
(ii) cytoplasmic aggregation of TDP-43,
(iii) decreased number of motor neurons in the spinal cord,
(iv) disruption of TDP-43 function in cryptic and alternative splicing,
(v) denervation of neuromuscular junctions,
(vi) a motor phenotype, and/or
(vii) early lethality.

30. The method of claim 29, wherein the candidate agent prevents or reduces cytoplasmic aggregation of TDP-43 and, optionally, restores nuclear localization of TDP-43.

31. A method of making a non-human animal model of TDP-43 proteinopathy comprising
(I) modifying the genome of a non-human animal to comprise:
(a) a mutated TARDBP gene at one chromosome at an endogenous TARDBP locus,
wherein the mutated TARDBP gene comprises a wildtype TARDBP gene sequence that comprises a mutation in a nuclear localization signal (NLS) encoding sequence or a prion like domain (PLD) encoding sequence such that the mutated TARDBP gene encodes a mutant TDP-43 polypeptide that lacks a functional NLS or a functional PLD, and
(b) a conditional knockout TARDBP gene at the other homologous chromosome at an endogenous TARDBP locus,
wherein the conditional knockout TARDBP gene comprises at least one exon flanked by a site-specific recombinase recognition sequence, and
(II) administering to the non-human animal a recombinase that recognizes the site-specific recombinase recognition sequence to create a knockout TARDBP gene from the conditional knockout TARDBP gene, wherein contacting the site-specific recombinase recognition sequence with the recombinase results in the deletion of at least one exon and formation of the knockout TARDBP gene,
wherein after the administering step, the non-human animal exhibits one or more TDP-43 proteinopathy characteristics in comparison to a control non-human animal.

32. The method of claim 31, wherein the one or more TDP-43 proteinopathy characteristics comprises:
(i) weight loss
(ii) cytoplasmic aggregation of TDP-43,
(iii) decreased number of motor neurons in the spinal cord,
(iv) disruption of TDP-43 function in cryptic and alternative splicing,
(v) denervation of neuromuscular junctions,
(vi) a motor phenotype, and/or
(vii) early lethality.

33. The method of claim 32, wherein the motor phenotype comprises one or more selected from the group consisting of: hind limb clasping, kyphosis, early hyperactivity, uncoordinated/ataxic movement, head wobbling, paralysis, and inability to right.

34. The method of claim 32, wherein the decreased number of motor neurons in the spinal cord comprises a decrease in the number of alpha motor neurons, but not gamma motor neurons.

35. The method of any one of claims 31-34, wherein each cell of the control non-human animal comprises:
(a) the mutated TARDBP gene at one chromosome at an endogenous TARDBP locus, and
(b) a wildtype TARDBP gene or the conditional knockout TARDBP gene at the other homologous chromosome at an endogenous TARDBP locus.

36. The method of any one of claims 31-35, wherein the step of administering takes place neonatally, and
wherein the non-human animal exhibits the one or more TDP-43 proteinopathy characteristics by, at, and/or around four to five weeks after the administering step, and/or at least two of the one or more TDP-43 proteinopathy characteristics by, at, and/or around seven to ten weeks after the administering step.

37. The method of any one of claims 31-35, wherein the step of administering takes place 1 month, 2 months, 3 months, 4 months, 5 months, or 6 months after birth of the non-human animal progeny, and
wherein the non-human animal progeny exhibits the one or more one or more TDP-43 proteinopathy characteristics 5-7 months after the administering step.

38. The method of any one of claims 31-37, wherein exon 3 of the conditional knockout TARDBP gene is flanked by the site-specific recombinase recognition sequence.

39. The method of any one of claims 31-38, wherein the site-specific recombinase recognition sequence comprises a loxP sequence and the recombinase is Cre recombinase.

40. The method of any one of claims 31-39, wherein the administering step comprises intraperitoneal or intracerebroventricular injection of a nucleic acid comprising a sequence that encodes the recombinase.

41. The method of any one of claims 31-40, wherein the administering step comprises intraperitoneal or intracerebroventricular injection of AAV particles comprising a nucleic acid comprising a sequence that encodes the recombinase,
wherein the nucleic acid further comprises:
(i) a promoter sequence that drives the expression of the recombinase,
(ii) a reporter gene sequence, optionally wherein the reporter gene sequence is operably linked to the recombinase gene sequence by a poly A sequence,
(iii) an adeno-associated virus (AAV) inverted terminal repeat (ITR) sequence at the 5′ and 3′ ends of the nucleic acid,
(iv) any combination of (i)-(iii).

42. The method of claim 41, wherein the AAV particles are AAV-PHP.eB particles.

43. The method of any one of claims 40-42, wherein the nucleic acid comprises the sequence set forth as SEQ ID NO:18 or SEQ ID NO:19.

44. The method of any one of claims 31-43, wherein the conditional knockout TARDBP gene comprises the wildtype TARDBP gene comprising a site-specific recombinase recognition sequence that flanks its exon 3.

45. The method of any one of claims 31-44, wherein the wildtype TARDBP gene is an endogenous wildtype TARDBP gene of the non-human animal.

46. The method of any one of claims 31-44, wherein the wildtype TARDBP gene is a wildtype human TARDBP gene.

47. The method of any one of claims 31-46, wherein the mutant TDP-43 polypeptide comprises:
(a) a point mutation of an amino acid in the NLS, or
(b) a deletion of at least a portion of the prion-like domain.

48. The method of claim 47, wherein
(a) the point mutation of an amino acid in the NLS comprises K82A K83A, R84A, K95A, K97A, K98A, or a combination thereof, and
(b) the deletion of at least a portion of the prion-like domain comprises a deletion of the amino acids at and between positions 274 and 414 of a wildtype TDP-43 polypeptide.

49. The method of any one of claims 31-48, wherein the mutant TDP-43 polypeptide comprises K82A K83A, R84A, K95A, K97A, and K98A point mutations.

50. The method of any one of claims 31-48, wherein the mutant TDP-43 polypeptide lacks the prion-like domain between and including the amino acids at positions 274 to 414 of a wildtype polypeptide.

51. The method of any one of claims 31-50, wherein modifying comprises replacing an endogenous TARDBP gene on one chromosome with the mutated TARDBP gene, and replacing an endogenous TARDBP gene at the other homologous chromosome with the conditional knockout TARDBP gene.

52. The method of any one of claims 31-51, wherein the non-human animal is a rat.

53. The method of any one of claims 31-51, wherein the non-human animal is a mouse.
